SEQUENCE LISTING (235 sequences)

SEQ ID NO: 1 - protein, 114 aa, Artificial Sequence
  Description of Artificial Sequence Synthetic polypeptide
EVQLQQSGAELVRPGASVNLSCTASGFNFKDDYMHWVKQRPEQGLEWIGWVDPENGDAAYASKFQGKATITADTSSNTAYLHLSSLTSEDAAVYYCITIFRSYWGQGTTLTVSS

SEQ ID NO: 2 - protein, 112 aa, Artificial Sequence
  Description of Artificial Sequence Synthetic polypeptide
DIQMTQSPLTLSVTIGQPASISCKSSQSLLDSDGETYLNWLLQRPGQSPKRLIYLVSKLDSGVPDRFTGSGSGTDFTLKISRVEAEDLGVYYCWQGTHFPLTFGGGTKLEIK

SEQ ID NO: 3 - protein, 5 aa, Artificial Sequence
  Description of Artificial Sequence Synthetic peptide
DDYMH

SEQ ID NO: 4 - protein, 17 aa, Artificial Sequence
  Description of Artificial Sequence Synthetic peptide
WVDPENGDAAYASKFQG

SEQ ID NO: 5 - protein, 5 aa, Artificial Sequence
  Description of Artificial Sequence Synthetic peptide
IFRSY

SEQ ID NO: 6 - protein, 16 aa, Artificial Sequence
  Description of Artificial Sequence Synthetic peptide
KSSQSLLDSDGETYLN

SEQ ID NO: 7 - protein, 7 aa, Artificial Sequence
  Description of Artificial Sequence Synthetic peptide
LVSKLDS

SEQ ID NO: 8 - protein, 9 aa, Artificial Sequence
  Description of Artificial Sequence Synthetic peptide
WQGTHFPLT

SEQ ID NO: 9 - protein, 7 aa, Artificial Sequence
  Description of Artificial Sequence Synthetic peptide
GFNFKDD

SEQ ID NO: 10 - protein, 6 aa, Artificial Sequence
  Description of Artificial Sequence Synthetic peptide
DPENGD

SEQ ID NO: 11 - protein, 5 aa, Artificial Sequence
  Description of Artificial Sequence Synthetic peptide
IFRSY

SEQ ID NO: 12 - protein, 16 aa, Artificial Sequence
  Description of Artificial Sequence Synthetic peptide
KSSQSLLDSDGETYLN

SEQ ID NO: 13 - protein, 7 aa, Artificial Sequence
  Description of Artificial Sequence Synthetic peptide
LVSKLDS

SEQ ID NO: 14 - protein, 9 aa, Artificial Sequence
  Description of Artificial Sequence Synthetic peptide
WQGTHFPLT

SEQ ID NO: 15 - protein, 8 aa, Artificial Sequence
  Description of Artificial Sequence Synthetic peptide
GFNFKDDY

SEQ ID NO: 16 - protein, 8 aa, Artificial Sequence
  Description of Artificial Sequence Synthetic peptide
VDPENGDA

SEQ ID NO: 17 - protein, 7 aa, Artificial Sequence
  Description of Artificial Sequence Synthetic peptide
ITIFRSY

SEQ ID NO: 18 - protein, 11 aa, Artificial Sequence
  Description of Artificial Sequence Synthetic peptide
QSLLDSDGETY

SEQ ID NO: 19 - protein, 3 aa, Artificial Sequence
  Description of Artificial Sequence Synthetic peptide
LVS

SEQ ID NO: 20 - protein, 9 aa, Artificial Sequence
  Description of Artificial Sequence Synthetic peptide
WQGTHFPLT

SEQ ID NO: 21 - protein, 10 aa, Artificial Sequence
  Description of Artificial Sequence Synthetic peptide
GFNFKDDYMH

SEQ ID NO: 22 - protein, 10 aa, Artificial Sequence
  Description of Artificial Sequence Synthetic peptide
WVDPENGDAA

SEQ ID NO: 23 - protein, 5 aa, Artificial Sequence
  Description of Artificial Sequence Synthetic peptide
IFRSY

SEQ ID NO: 24 - protein, 16 aa, Artificial Sequence
  Description of Artificial Sequence Synthetic peptide
KSSQSLLDSDGETYLN

SEQ ID NO: 25 - protein, 7 aa, Artificial Sequence
  Description of Artificial Sequence Synthetic peptide
LVSKLDS

SEQ ID NO: 26 - protein, 9 aa, Artificial Sequence
  Description of Artificial Sequence Synthetic peptide
WQGTHFPLT

SEQ ID NO: 27 - protein, 114 aa, Artificial Sequence; identical to SEQ ID NO: 1
  Description of Artificial Sequence Synthetic polypeptide

SEQ ID NO: 28 - protein, 112 aa, Artificial Sequence
  Description of Artificial Sequence Synthetic polypeptide
DIQMTQTPLTLSVTIGQPASISCKSSQSLLDSDGETYLNWLLQRPGQSPKRLIYLVSKLDSGVPDRFTGSGSGTDFTLKISRVEAEDLGVYYCWQGTHFPLTFGGGTKLEIK

SEQ ID NO: 29 - protein, 5 aa, Artificial Sequence
  Description of Artificial Sequence Synthetic peptide
DDYMH

SEQ ID NO: 30 - protein, 17 aa, Artificial Sequence
  Description of Artificial Sequence Synthetic peptide
WVDPENGDAAYASKFQG

SEQ ID NO: 31 - protein, 5 aa, Artificial Sequence
  Description of Artificial Sequence Synthetic peptide
IFRSY

SEQ ID NO: 32 - protein, 16 aa, Artificial Sequence
  Description of Artificial Sequence Synthetic peptide
KSSQSLLDSDGETYLN

SEQ ID NO: 33 - protein, 7 aa, Artificial Sequence
  Description of Artificial Sequence Synthetic peptide
LVSKLDS

SEQ ID NO: 34 - protein, 9 aa, Artificial Sequence
  Description of Artificial Sequence Synthetic peptide
WQGTHFPLT

SEQ ID NO: 35 - protein, 7 aa, Artificial Sequence
  Description of Artificial Sequence Synthetic peptide
GFNFKDD

SEQ ID NO: 36 - protein, 6 aa, Artificial Sequence
  Description of Artificial Sequence Synthetic peptide
DPENGD

SEQ ID NO: 37 - protein, 5 aa, Artificial Sequence
  Description of Artificial Sequence Synthetic peptide
IFRSY

SEQ ID NO: 38 - protein, 16 aa, Artificial Sequence
  Description of Artificial Sequence Synthetic peptide
KSSQSLLDSDGETYLN

SEQ ID NO: 39 - protein, 7 aa, Artificial Sequence
  Description of Artificial Sequence Synthetic peptide
LVSKLDS

SEQ ID NO: 40 - protein, 9 aa, Artificial Sequence
  Description of Artificial Sequence Synthetic peptide
WQGTHFPLT

SEQ ID NO: 41 - protein, 8 aa, Artificial Sequence
  Description of Artificial Sequence Synthetic peptide
GFNFKDDY

SEQ ID NO: 42 - protein, 8 aa, Artificial Sequence
  Description of Artificial Sequence Synthetic peptide
VDPENGDA

SEQ ID NO: 43 - protein, 7 aa, Artificial Sequence
  Description of Artificial Sequence Synthetic peptide
ITIFRSY

SEQ ID NO: 44 - protein, 11 aa, Artificial Sequence
  Description of Artificial Sequence Synthetic peptide
QSLLDSDGETY

SEQ ID NO: 45 - protein, 3 aa, Artificial Sequence
  Description of Artificial Sequence Synthetic peptide
LVS

SEQ ID NO: 46 - protein, 9 aa, Artificial Sequence
  Description of Artificial Sequence Synthetic peptide
WQGTHFPLT

SEQ ID NO: 47 - protein, 10 aa, Artificial Sequence
  Description of Artificial Sequence Synthetic peptide
GFNFKDDYMH

SEQ ID NO: 48 - protein, 10 aa, Artificial Sequence
  Description of Artificial Sequence Synthetic peptide
WVDPENGDAA

SEQ ID NO: 49 - protein, 5 aa, Artificial Sequence
  Description of Artificial Sequence Synthetic peptide
IFRSY

SEQ ID NO: 50 - protein, 16 aa, Artificial Sequence
  Description of Artificial Sequence Synthetic peptide
KSSQSLLDSDGETYLN

SEQ ID NO: 51 - protein, 7 aa, Artificial Sequence
  Description of Artificial Sequence Synthetic peptide
LVSKLDS

SEQ ID NO: 52 - protein, 9 aa, Artificial Sequence
  Description of Artificial Sequence Synthetic peptide
WQGTHFPLT

SEQ ID NO: 53 - protein, 124 aa, Artificial Sequence
  Description of Artificial Sequence Synthetic polypeptide
DVQLQESGAELVKPGASVILSCKASGYTFTDYTIHWLKQTSGQGLEWIGWFYPGSGSVKYNEKYNEKFRDKATLTADKSSRTVYMELIRLTSEDSAVYFCARHGSYYDDAMDYWGQGTTVTVSS

SEQ ID NO: 54 - protein, 112 aa, Artificial Sequence
  Description of Artificial Sequence Synthetic polypeptide
DVQMIQSPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIK

SEQ ID NO: 55 - protein, 5 aa, Artificial Sequence
  Description of Artificial Sequence Synthetic peptide
DYTIH

SEQ ID NO: 56 - protein, 21 aa, Artificial Sequence
  Description of Artificial Sequence Synthetic peptide
WFYPGSGSVKYNEKYNEKFRD

SEQ ID NO: 57 - protein, 11 aa, Artificial Sequence
  Description of Artificial Sequence Synthetic peptide
HGSYYDDAMDY

SEQ ID NO: 58 - protein, 16 aa, Artificial Sequence
  Description of Artificial Sequence Synthetic peptide
RSSQNIVHSNGNTYLE

SEQ ID NO: 59 - protein, 7 aa, Artificial Sequence
  Description of Artificial Sequence Synthetic peptide
RVSKRFS

SEQ ID NO: 60 - protein, 9 aa, Artificial Sequence
  Description of Artificial Sequence Synthetic peptide
FQGSHVPWT

SEQ ID NO: 61 - protein, 7 aa, Artificial Sequence
  Description of Artificial Sequence Synthetic peptide
GYTFTDY

SEQ ID NO: 62 - protein, 10 aa, Artificial Sequence
  Description of Artificial Sequence Synthetic peptide
YPGSGSVKYN

SEQ ID NO: 63 - protein, 11 aa, Artificial Sequence
  Description of Artificial Sequence Synthetic peptide
HGSYYDDAMDY

SEQ ID NO: 64 - protein, 16 aa, Artificial Sequence
  Description of Artificial Sequence Synthetic peptide
RSSQNIVHSNGNTYLE

SEQ ID NO: 65 - protein, 7 aa, Artificial Sequence
  Description of Artificial Sequence Synthetic peptide
RVSKRFS

SEQ ID NO: 66 - protein, 9 aa, Artificial Sequence
  Description of Artificial Sequence Synthetic peptide
FQGSHVPWT

SEQ ID NO: 67 - protein, 8 aa, Artificial Sequence
  Description of Artificial Sequence Synthetic peptide
GYTFTDYT

SEQ ID NO: 68 - protein, 12 aa, Artificial Sequence
  Description of Artificial Sequence Synthetic peptide
FYPGSGSVKYNE

SEQ ID NO: 69 - protein, 13 aa, Artificial Sequence
  Description of Artificial Sequence Synthetic peptide
ARHGSYYDDAMDY

SEQ ID NO: 70 - protein, 11 aa, Artificial Sequence
  Description of Artificial Sequence Synthetic peptide
QNIVHSNGNTY

SEQ ID NO: 71 - protein, 3 aa, Artificial Sequence
  Description of Artificial Sequence Synthetic peptide
RVS

SEQ ID NO: 72 - protein, 9 aa, Artificial Sequence
  Description of Artificial Sequence Synthetic peptide
FQGSHVPWT

SEQ ID NO: 73 - protein, 10 aa, Artificial Sequence
  Description of Artificial Sequence Synthetic peptide
GYTFTDYTIH

SEQ ID NO: 74 - protein, 14 aa, Artificial Sequence
  Description of Artificial Sequence Synthetic peptide
WFYPGSGSVKYNEK

SEQ ID NO: 75 - protein, 11 aa, Artificial Sequence
  Description of Artificial Sequence Synthetic peptide
HGSYYDDAMDY

SEQ ID NO: 76 - protein, 16 aa, Artificial Sequence
  Description of Artificial Sequence Synthetic peptide
RSSQNIVHSNGNTYLE

SEQ ID NO: 77 - protein, 7 aa, Artificial Sequence
  Description of Artificial Sequence Synthetic peptide
RVSKRFS

SEQ ID NO: 78 - protein, 9 aa, Artificial Sequence
  Description of Artificial Sequence Synthetic peptide
FQGSHVPWT

SEQ ID NO: 79 - protein, 124 aa, Artificial Sequence; identical to SEQ ID NO: 53
  Description of Artificial Sequence Synthetic polypeptide

SEQ ID NO: 80 - protein, 112 aa, Artificial Sequence
  Description of Artificial Sequence Synthetic polypeptide
DIVMTQTPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIK

SEQ ID NO: 81 - protein, 5 aa, Artificial Sequence
  Description of Artificial Sequence Synthetic peptide
DYTIH

SEQ ID NO: 82 - protein, 21 aa, Artificial Sequence
  Description of Artificial Sequence Synthetic peptide
WFYPGSGSVKYNEKYNEKFRD

SEQ ID NO: 83 - protein, 11 aa, Artificial Sequence
  Description of Artificial Sequence Synthetic peptide
HGSYYDDAMDY

SEQ ID NO: 84 - protein, 16 aa, Artificial Sequence
  Description of Artificial Sequence Synthetic peptide
RSSQNIVHSNGNTYLE

SEQ ID NO: 85 - protein, 7 aa, Artificial Sequence
  Description of Artificial Sequence Synthetic peptide
RVSKRFS

SEQ ID NO: 86 - protein, 9 aa, Artificial Sequence
  Description of Artificial Sequence Synthetic peptide
FQGSHVPWT

SEQ ID NO: 87 - protein, 7 aa, Artificial Sequence
  Description of Artificial Sequence Synthetic peptide
GYTFTDY

SEQ ID NO: 88 - protein, 10 aa, Artificial Sequence
  Description of Artificial Sequence Synthetic peptide
YPGSGSVKYN

SEQ ID NO: 89 - protein, 11 aa, Artificial Sequence
  Description of Artificial Sequence Synthetic peptide
HGSYYDDAMDY

SEQ ID NO: 90 - protein, 16 aa, Artificial Sequence
  Description of Artificial Sequence Synthetic peptide
RSSQNIVHSNGNTYLE

SEQ ID NO: 91 - protein, 7 aa, Artificial Sequence
  Description of Artificial Sequence Synthetic peptide
RVSKRFS

SEQ ID NO: 92 - protein, 9 aa, Artificial Sequence
  Description of Artificial Sequence Synthetic peptide
FQGSHVPWT

SEQ ID NO: 93 - protein, 8 aa, Artificial Sequence
  Description of Artificial Sequence Synthetic peptide
GYTFTDYT

SEQ ID NO: 94 - protein, 12 aa, Artificial Sequence
  Description of Artificial Sequence Synthetic peptide
FYPGSGSVKYNE

SEQ ID NO: 95 - protein, 13 aa, Artificial Sequence
  Description of Artificial Sequence Synthetic peptide
ARHGSYYDDAMDY

SEQ ID NO: 96 - protein, 11 aa, Artificial Sequence
  Description of Artificial Sequence Synthetic peptide
QNIVHSNGNTY

SEQ ID NO: 97 - protein, 3 aa, Artificial Sequence
  Description of Artificial Sequence Synthetic peptide
RVS

SEQ ID NO: 98 - protein, 9 aa, Artificial Sequence
  Description of Artificial Sequence Synthetic peptide
FQGSHVPWT

SEQ ID NO: 99 - protein, 10 aa, Artificial Sequence
  Description of Artificial Sequence Synthetic peptide
GYTFTDYTIH

SEQ ID NO: 100 - protein, 14 aa, Artificial Sequence
  Description of Artificial Sequence Synthetic peptide
WFYPGSGSVKYNEK

SEQ ID NO: 101 - protein, 11 aa, Artificial Sequence
  Description of Artificial Sequence Synthetic peptide
HGSYYDDAMDY

SEQ ID NO: 102 - protein, 16 aa, Artificial Sequence
  Description of Artificial Sequence Synthetic peptide
RSSQNIVHSNGNTYLE

SEQ ID NO: 103 - protein, 7 aa, Artificial Sequence
  Description of Artificial Sequence Synthetic peptide
RVSKRFS

SEQ ID NO: 104 - protein, 9 aa, Artificial Sequence
  Description of Artificial Sequence Synthetic peptide
FQGSHVPWT

SEQ ID NO: 105 - protein, 114 aa, Artificial Sequence
  Description of Artificial Sequence Synthetic polypeptide
EVQLQQSGAELVRPGASVNLSCTASGFNFKDDYMHWVKQRPEQGLEWIGWIDPENGDAAYASKFQGKATITADTSSNTAYLQLSSLTSEDTAVYYCITIFRSYWGQGTTLTVSS

SEQ ID NO: 106 - protein, 112 aa, Artificial Sequence; identical to SEQ ID NO: 2
  Description of Artificial Sequence Synthetic polypeptide

SEQ ID NO: 107 - protein, 5 aa, Artificial Sequence
  Description of Artificial Sequence Synthetic peptide
DDYMH

SEQ ID NO: 108 - protein, 17 aa, Artificial Sequence
  Description of Artificial Sequence Synthetic peptide
WIDPENGDAAYASKFQG

SEQ ID NO: 109 - protein, 5 aa, Artificial Sequence
  Description of Artificial Sequence Synthetic peptide
IFRSY

SEQ ID NO: 110 - protein, 16 aa, Artificial Sequence
  Description of Artificial Sequence Synthetic peptide
KSSQSLLDSDGETYLN

SEQ ID NO: 111 - protein, 7 aa, Artificial Sequence
  Description of Artificial Sequence Synthetic peptide
LVSKLDS

SEQ ID NO: 112 - protein, 9 aa, Artificial Sequence
  Description of Artificial Sequence Synthetic peptide
WQGTHFPLT

SEQ ID NO: 113 - protein, 7 aa, Artificial Sequence
  Description of Artificial Sequence Synthetic peptide
GFNFKDD

SEQ ID NO: 114 - protein, 6 aa, Artificial Sequence
  Description of Artificial Sequence Synthetic peptide
DPENGD

SEQ ID NO: 115 - protein, 5 aa, Artificial Sequence
  Description of Artificial Sequence Synthetic peptide
IFRSY

SEQ ID NO: 116 - protein, 16 aa, Artificial Sequence
  Description of Artificial Sequence Synthetic peptide
KSSQSLLDSDGETYLN

SEQ ID NO: 117 - protein, 7 aa, Artificial Sequence
  Description of Artificial Sequence Synthetic peptide
LVSKLDS

SEQ ID NO: 118 - protein, 9 aa, Artificial Sequence
  Description of Artificial Sequence Synthetic peptide
WQGTHFPLT

SEQ ID NO: 119 - protein, 8 aa, Artificial Sequence
  Description of Artificial Sequence Synthetic peptide
GFNFKDDY

SEQ ID NO: 120 - protein, 8 aa, Artificial Sequence
  Description of Artificial Sequence Synthetic peptide
IDPENGDA

SEQ ID NO: 121 - protein, 7 aa, Artificial Sequence
  Description of Artificial Sequence Synthetic peptide
ITIFRSY

SEQ ID NO: 122 - protein, 11 aa, Artificial Sequence
  Description of Artificial Sequence Synthetic peptide
QSLLDSDGETY

SEQ ID NO: 123 - protein, 3 aa, Artificial Sequence
  Description of Artificial Sequence Synthetic peptide
LVS

SEQ ID NO: 124 - protein, 9 aa, Artificial Sequence
  Description of Artificial Sequence Synthetic peptide
WQGTHFPLT

SEQ ID NO: 125 - protein, 10 aa, Artificial Sequence
  Description of Artificial Sequence Synthetic peptide
GFNFKDDYMH

SEQ ID NO: 126 - protein, 10 aa, Artificial Sequence
  Description of Artificial Sequence Synthetic peptide
WIDPENGDAA

SEQ ID NO: 127 - protein, 5 aa, Artificial Sequence
  Description of Artificial Sequence Synthetic peptide
IFRSY

SEQ ID NO: 128 - protein, 16 aa, Artificial Sequence
  Description of Artificial Sequence Synthetic peptide
KSSQSLLDSDGETYLN

SEQ ID NO: 129 - protein, 7 aa, Artificial Sequence
  Description of Artificial Sequence Synthetic peptide
LVSKLDS

SEQ ID NO: 130 - protein, 9 aa, Artificial Sequence
  Description of Artificial Sequence Synthetic peptide
WQGTHFPLT

SEQ ID NO: 131 - protein, 124 aa, Artificial Sequence
  Description of Artificial Sequence Synthetic polypeptide
QIQLVQSGAELVKPGASVILSCKASGYTFTDYTIHWLKQTSGQGLEWIGWFYPGSGSVKYNEKYNEKFRDKATLTADKSSRTVYMELIRLTSEDSAVYFCARHGSYYDDAMDYWGQGTTLTVSS

SEQ ID NO: 132 - protein, 112 aa, Artificial Sequence; identical to SEQ ID NO: 80
  Description of Artificial Sequence Synthetic polypeptide

SEQ ID NO: 133 - protein, 5 aa, Artificial Sequence
  Description of Artificial Sequence Synthetic peptide
DYTIH

SEQ ID NO: 134 - protein, 21 aa, Artificial Sequence
  Description of Artificial Sequence Synthetic peptide
WFYPGSGSVKYNEKYNEKFRD

SEQ ID NO: 135 - protein, 11 aa, Artificial Sequence
  Description of Artificial Sequence Synthetic peptide
HGSYYDDAMDY

SEQ ID NO: 136 - protein, 16 aa, Artificial Sequence
  Description of Artificial Sequence Synthetic peptide
RSSQNIVHSNGNTYLE

SEQ ID NO: 137 - protein, 7 aa, Artificial Sequence
  Description of Artificial Sequence Synthetic peptide
RVSKRFS

SEQ ID NO: 138 - protein, 9 aa, Artificial Sequence
  Description of Artificial Sequence Synthetic peptide
FQGSHVPWT

SEQ ID NO: 139 - protein, 7 aa, Artificial Sequence
  Description of Artificial Sequence Synthetic peptide
GYTFTDY

SEQ ID NO: 140 - protein, 10 aa, Artificial Sequence
  Description of Artificial Sequence Synthetic peptide
YPGSGSVKYN

SEQ ID NO: 141 - protein, 11 aa, Artificial Sequence
  Description of Artificial Sequence Synthetic peptide
HGSYYDDAMDY

SEQ ID NO: 142 - protein, 16 aa, Artificial Sequence
  Description of Artificial Sequence Synthetic peptide
RSSQNIVHSNGNTYLE

SEQ ID NO: 143 - protein, 7 aa, Artificial Sequence
  Description of Artificial Sequence Synthetic peptide
RVSKRFS

SEQ ID NO: 144 - protein, 9 aa, Artificial Sequence
  Description of Artificial Sequence Synthetic peptide
FQGSHVPWT

SEQ ID NO: 145 - protein, 8 aa, Artificial Sequence
  Description of Artificial Sequence Synthetic peptide
GYTFTDYT

SEQ ID NO: 146 - protein, 12 aa, Artificial Sequence
  Description of Artificial Sequence Synthetic peptide
FYPGSGSVKYNE

SEQ ID NO: 147 - protein, 13 aa, Artificial Sequence
  Description of Artificial Sequence Synthetic peptide
ARHGSYYDDAMDY

SEQ ID NO: 148 - protein, 11 aa, Artificial Sequence
  Description of Artificial Sequence Synthetic peptide
QNIVHSNGNTY

SEQ ID NO: 149 - protein, 3 aa, Artificial Sequence
  Description of Artificial Sequence Synthetic peptide
RVS

SEQ ID NO: 150 - protein, 9 aa, Artificial Sequence
  Description of Artificial Sequence Synthetic peptide
FQGSHVPWT

SEQ ID NO: 151 - protein, 10 aa, Artificial Sequence
  Description of Artificial Sequence Synthetic peptide
GYTFTDYTIH

SEQ ID NO: 152 - protein, 14 aa, Artificial Sequence
  Description of Artificial Sequence Synthetic peptide
WFYPGSGSVKYNEK

SEQ ID NO: 153 - protein, 11 aa, Artificial Sequence
  Description of Artificial Sequence Synthetic peptide
HGSYYDDAMDY

SEQ ID NO: 154 - protein, 16 aa, Artificial Sequence
  Description of Artificial Sequence Synthetic peptide
RSSQNIVHSNGNTYLE

SEQ ID NO: 155 - protein, 7 aa, Artificial Sequence
  Description of Artificial Sequence Synthetic peptide
RVSKRFS

SEQ ID NO: 156 - protein, 9 aa, Artificial Sequence
  Description of Artificial Sequence Synthetic peptide
FQGSHVPWT

SEQ ID NO: 157 - protein, 124 aa, Artificial Sequence; identical to SEQ ID NO: 131
  Description of Artificial Sequence Synthetic polypeptide

SEQ ID NO: 158 - protein, 112 aa, Artificial Sequence
  Description of Artificial Sequence Synthetic polypeptide
DVVMTQTPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIK

SEQ ID NO: 159 - protein, 5 aa, Artificial Sequence
  Description of Artificial Sequence Synthetic peptide
DYTIH

SEQ ID NO: 160 - protein, 21 aa, Artificial Sequence
  Description of Artificial Sequence Synthetic peptide
WFYPGSGSVKYNEKYNEKFRD

SEQ ID NO: 161 - protein, 11 aa, Artificial Sequence
  Description of Artificial Sequence Synthetic peptide
HGSYYDDAMDY

SEQ ID NO: 162 - protein, 16 aa, Artificial Sequence
  Description of Artificial Sequence Synthetic peptide
RSSQNIVHSNGNTYLE

SEQ ID NO: 163 - protein, 7 aa, Artificial Sequence
  Description of Artificial Sequence Synthetic peptide
RVSKRFS

SEQ ID NO: 164 - protein, 9 aa, Artificial Sequence
  Description of Artificial Sequence Synthetic peptide
FQGSHVPWT

SEQ ID NO: 165 - protein, 7 aa, Artificial Sequence
  Description of Artificial Sequence Synthetic peptide
GYTFTDY

SEQ ID NO: 166 - protein, 10 aa, Artificial Sequence
  Description of Artificial Sequence Synthetic peptide
YPGSGSVKYN

SEQ ID NO: 167 - protein, 11 aa, Artificial Sequence
  Description of Artificial Sequence Synthetic peptide
HGSYYDDAMDY

SEQ ID NO: 168 - protein, 16 aa, Artificial Sequence
  Description of Artificial Sequence Synthetic peptide
RSSQNIVHSNGNTYLE

SEQ ID NO: 169 - protein, 7 aa, Artificial Sequence
  Description of Artificial Sequence Synthetic peptide
RVSKRFS

SEQ ID NO: 170 - protein, 9 aa, Artificial Sequence
  Description of Artificial Sequence Synthetic peptide
FQGSHVPWT

SEQ ID NO: 171 - protein, 8 aa, Artificial Sequence
  Description of Artificial Sequence Synthetic peptide
GYTFTDYT

SEQ ID NO: 172 - protein, 12 aa, Artificial Sequence
  Description of Artificial Sequence Synthetic peptide
FYPGSGSVKYNE

SEQ ID NO: 173 - protein, 13 aa, Artificial Sequence
  Description of Artificial Sequence Synthetic peptide
ARHGSYYDDAMDY

SEQ ID NO: 174 - protein, 11 aa, Artificial Sequence
  Description of Artificial Sequence Synthetic peptide
QNIVHSNGNTY

SEQ ID NO: 175 - protein, 3 aa, Artificial Sequence
  Description of Artificial Sequence Synthetic peptide
RVS

SEQ ID NO: 176 - protein, 9 aa, Artificial Sequence
  Description of Artificial Sequence Synthetic peptide
FQGSHVPWT

SEQ ID NO: 177 - protein, 10 aa, Artificial Sequence
  Description of Artificial Sequence Synthetic peptide
GYTFTDYTIH

SEQ ID NO: 178 - protein, 14 aa, Artificial Sequence
  Description of Artificial Sequence Synthetic peptide
WFYPGSGSVKYNEK

SEQ ID NO: 179 - protein, 11 aa, Artificial Sequence
  Description of Artificial Sequence Synthetic peptide
HGSYYDDAMDY

SEQ ID NO: 180 - protein, 16 aa, Artificial Sequence
  Description of Artificial Sequence Synthetic peptide
RSSQNIVHSNGNTYLE

SEQ ID NO: 181 - protein, 7 aa, Artificial Sequence
  Description of Artificial Sequence Synthetic peptide
RVSKRFS

SEQ ID NO: 182 - protein, 9 aa, Artificial Sequence
  Description of Artificial Sequence Synthetic peptide
FQGSHVPWT

SEQ ID NO: 183 - protein, 120 aa, Artificial Sequence
  Description of Artificial Sequence Synthetic polypeptide
QVQLQQSGGGLVQPGGSLKLSCAASGFTFSNYYMSWARQTPEKRLEWVAYISNDGDATYYPDSVKGRFTISRDNAKNTLYLQMSSLKSEDTAMYYCTREDYYSDYWFASWGQGTTVTVSS

SEQ ID NO: 184 - protein, 107 aa, Artificial Sequence
  Description of Artificial Sequence Synthetic polypeptide
DIVMTQSQKFMSTTIEDRVSITCKASQNVGTAVAWYQQKPGQSPKLLIYSTSNRYTGVPDRFTGSGSGTDFTLTISNIQSEDLADYFCQQYSSYPLTFGGGTKLEIK

SEQ ID NO: 185 - protein, 5 aa, Artificial Sequence
  Description of Artificial Sequence Synthetic peptide
NYYMS

SEQ ID NO: 186 - protein, 17 aa, Artificial Sequence
  Description of Artificial Sequence Synthetic peptide
YISNDGDATYYPDSVKG

SEQ ID NO: 187 - protein, 11 aa, Artificial Sequence
  Description of Artificial Sequence Synthetic peptide
EDYYSDYWFAS

SEQ ID NO: 188 - protein, 11 aa, Artificial Sequence
  Description of Artificial Sequence Synthetic peptide
KASQNVGTAVA

SEQ ID NO: 189 - protein, 7 aa, Artificial Sequence
  Description of Artificial Sequence Synthetic peptide
STSNRYT

SEQ ID NO: 190 - protein, 9 aa, Artificial Sequence
  Description of Artificial Sequence Synthetic peptide
QQYSSYPLT

SEQ ID NO: 191 - protein, 7 aa, Artificial Sequence
  Description of Artificial Sequence Synthetic peptide
GFTFSNY

SEQ ID NO: 192 - protein, 6 aa, Artificial Sequence
  Description of Artificial Sequence Synthetic peptide
SNDGDA

SEQ ID NO: 193 - protein, 11 aa, Artificial Sequence
  Description of Artificial Sequence Synthetic peptide
EDYYSDYWFAS

SEQ ID NO: 194 - protein, 11 aa, Artificial Sequence
  Description of Artificial Sequence Synthetic peptide
KASQNVGTAVA

SEQ ID NO: 195 - protein, 7 aa, Artificial Sequence
  Description of Artificial Sequence Synthetic peptide
STSNRYT

SEQ ID NO: 196 - protein, 9 aa, Artificial Sequence
  Description of Artificial Sequence Synthetic peptide
QQYSSYPLT

SEQ ID NO: 197 - protein, 8 aa, Artificial Sequence
  Description of Artificial Sequence Synthetic peptide
GFTFSNYY

SEQ ID NO: 198 - protein, 8 aa, Artificial Sequence
  Description of Artificial Sequence Synthetic peptide
ISNDGDAT

SEQ ID NO: 199 - protein, 13 aa, Artificial Sequence
  Description of Artificial Sequence Synthetic peptide
TREDYYSDYWFAS

SEQ ID NO: 200 - protein, 6 aa, Artificial Sequence
  Description of Artificial Sequence Synthetic peptide
QNVGTA

SEQ ID NO: 201 - protein, 3 aa, Artificial Sequence
  Description of Artificial Sequence Synthetic peptide
STS

SEQ ID NO: 202 - protein, 9 aa, Artificial Sequence
  Description of Artificial Sequence Synthetic peptide
QQYSSYPLT

SEQ ID NO: 203 - protein, 10 aa, Artificial Sequence
  Description of Artificial Sequence Synthetic peptide
GFTFSNYYMS

SEQ ID NO: 204 - protein, 10 aa, Artificial Sequence
  Description of Artificial Sequence Synthetic peptide
YISNDGDATY

SEQ ID NO: 205 - protein, 11 aa, Artificial Sequence
  Description of Artificial Sequence Synthetic peptide
EDYYSDYWFAS

SEQ ID NO: 206 - protein, 11 aa, Artificial Sequence
  Description of Artificial Sequence Synthetic peptide
KASQNVGTAVA

SEQ ID NO: 207 - protein, 7 aa, Artificial Sequence
  Description of Artificial Sequence Synthetic peptide
STSNRYT

SEQ ID NO: 208 - protein, 9 aa, Artificial Sequence
  Description of Artificial Sequence Synthetic peptide
QQYSSYPLT

SEQ ID NO: 209 - protein, 44 aa, Artificial Sequence
  Description of Artificial Sequence Synthetic polypeptide
TTPAPRPPTPAPTIASQPLSLRPEACRPAAGGAVHTRGLDFACD

SEQ ID NO: 210 - protein, 44 aa, Artificial Sequence
  Description of Artificial Sequence Synthetic polypeptide
STPAPRPPTPAPTIASQPLSLRPEACRPAAGGAVHTRGLDFACD

SEQ ID NO: 211 - protein, 214 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 212 - protein, 189 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 213 - protein, 161 aa, Artificial Sequence
  Description of Artificial Sequence Synthetic polypeptide
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 214 - protein, 152 aa, Artificial Sequence
  Description of Artificial Sequence Synthetic polypeptide
NSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 215 - protein, 444 aa, Artificial Sequence
  Description of Artificial Sequence Synthetic polypeptide
EVQLQQSGAELVRPGASVNLSCTASGFNFKDDYMHWVKQRPEQGLEWIGWVDPENGDAAYASKFQGKATITADTSSNTAYLHLSSLTSEDAAVYYCITIFRSYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 219 aa, Artificial Sequence
  Description of Artificial Sequence Synthetic polypeptide
DIQMTQSPLTLSVTIGQPASISCKSSQSLLDSDGETYLNWLLQRPGQSPKRLIYLVSKLDSGVPDRFTGSGSGTDFTLKISRVEAEDLGVYYCWQGTHFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 444 aa, Artificial Sequence; identical to SEQ ID NO: 215
  Description of Artificial Sequence Synthetic polypeptide

SEQ ID NO: 218 - protein, 219 aa, Artificial Sequence
  Description of Artificial Sequence Synthetic polypeptide
DIQMTQTPLTLSVTIGQPASISCKSSQSLLDSDGETYLNWLLQRPGQSPKRLIYLVSKLDSGVPDRFTGSGSGTDFTLKISRVEAEDLGVYYCWQGTHFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 454 aa, Artificial Sequence
  Description of Artificial Sequence Synthetic polypeptide
DVQLQESGAELVKPGASVILSCKASGYTFTDYTIHWLKQTSGQGLEWIGWFYPGSGSVKYNEKYNEKFRDKATLTADKSSRTVYMELIRLTSEDSAVYFCARHGSYYDDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 219 aa, Artificial Sequence
  Description of Artificial Sequence Synthetic polypeptide
DVQMIQSPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 454 aa, Artificial Sequence; identical to SEQ ID NO: 219
  Description of Artificial Sequence Synthetic polypeptide

SEQ ID NO: 222 - protein, 219 aa, Artificial Sequence
  Description of Artificial Sequence Synthetic polypeptide
DIVMTQTPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 444 aa, Artificial Sequence
  Description of Artificial Sequence Synthetic polypeptide
EVQLQQSGAELVRPGASVNLSCTASGFNFKDDYMHWVKQRPEQGLEWIGWIDPENGDAAYASKFQGKATITADTSSNTAYLQLSSLTSEDTAVYYCITIFRSYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 219 aa, Artificial Sequence; identical to SEQ ID NO: 216
  Description of Artificial Sequence Synthetic polypeptide

SEQ ID NO: 225 - protein, 454 aa, Artificial Sequence
  Description of Artificial Sequence Synthetic polypeptide
QIQLVQSGAELVKPGASVILSCKASGYTFTDYTIHWLKQTSGQGLEWIGWFYPGSGSVKYNEKYNEKFRDKATLTADKSSRTVYMELIRLTSEDSAVYFCARHGSYYDDAMDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 219 aa, Artificial Sequence; identical to SEQ ID NO: 222
  Description of Artificial Sequence Synthetic polypeptide

SEQ ID NO: 227 - protein, 454 aa, Artificial Sequence; identical to SEQ ID NO: 225
  Description of Artificial Sequence Synthetic polypeptide

SEQ ID NO: 228 - protein, 219 aa, Artificial Sequence
  Description of Artificial Sequence Synthetic polypeptide
DVVMTQTPLSLPVSLGDQASISCRSSQNIVHSNGNTYLEWYLQKPGQSPKLLIYRVSKRFSGVPDRFSGSGSGTDFTLKITRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 450 aa, Artificial Sequence
  Description of Artificial Sequence Synthetic polypeptide
QVQLQQSGGGLVQPGGSLKLSCAASGFTFSNYYMSWARQTPEKRLEWVAYISNDGDATYYPDSVKGRFTISRDNAKNTLYLQMSSLKSEDTAMYYCTREDYYSDYWFASWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 214 aa, Artificial Sequence
  Description of Artificial Sequence Synthetic polypeptide
DIVMTQSQKFMSTTIEDRVSITCKASQNVGTAVAWYQQKPGQSPKLLIYSTSNRYTGVPDRFTGSGSGTDFTLTISNIQSEDLADYFCQQYSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 5 aa, Artificial Sequence
  Description of Artificial Sequence Synthetic peptide
GSGGS

SEQ ID NO: 232 - protein, 4 aa, Artificial Sequence
  Description of Artificial Sequence Synthetic peptide
GGGS

SEQ ID NO: 233 - protein, 5 aa, Artificial Sequence
  Description of Artificial Sequence Synthetic peptide
GGGGS

SEQ ID NO: 234 - protein, 6 aa, Artificial Sequence
  Description of Artificial Sequence Synthetic 6xHis tag
HHHHHH

SEQ ID NO: 235 - protein, 8 aa, Artificial Sequence
  Description of Artificial Sequence Synthetic peptide
DYKDDDDK

(19) United States
(12) Patent Application Publication
(10) Pub. No.: US 2023/0174651 A1
(43) Pub. Date: Jun. 8, 2023

(54) MATERIALS AND METHODS FOR HINGE REGIONS IN FUNCTIONAL EXOGENOUS RECEPTORS

(71) Applicant: Janssen Biotech, Inc., Horsham, PA (US)

(72) Inventors: Jun CHEN, New Hope, PA (US); Martin Jack BORROK, III, Chalfont, PA (US); Rajkumar GANESAN, Blue Bell, PA (US); Degang SONG, Wynnewood, PA (US); Shixue SHEN, Flourtown, PA (US); Bonnie WU, Malvern, PA (US)

(21) Appl. No.: 17/844,484

(22) Filed: Jun. 20, 2022

Related U.S. Application Data
(60) Provisional application No. 63/214,202, filed on Jun. 23, 2021.
(60) Provisional application No. 63/214,211, filed on Jun. 23, 2021.
(60) Provisional application No. 63/214,209, filed on Jun. 23, 2021.
(60) Provisional application No. 63/214,216, filed on Jun. 23, 2021.
(60) Provisional application No. 63/214,219, filed on Jun. 23, 2021.

(51) Int. Cl. C07K 16/28; G01N 33/50; G01N 33/68

(52) U.S. Cl. CPC C07K 16/2815; G01N 33/5005; G01N 33/68; C07K 2317/622; C07K 2317/565; C07K 2317/24; C07K 2317/21; G01N 2333/70517

(57) ABSTRACT

An antibody that binds a CD8α hinge region, wherein the antibody is capable of binding to a functional exogenous receptor, and wherein the functional exogenous receptor comprises an extracellular domain, the CD8α hinge region, a transmembrane domain, and an intracellular signaling domain.

### CROSS-REFERENCE TO RELATED APPLICATIONS

This application claims the benefit of U.S. Ser. No. 63/214,202 filed Jun. 23, 2021; U.S. Ser. No. 63/214,211 filed Jun. 23, 2021; U.S. Ser. No. 63/214,209 filed Jun. 23, 2021; U.S. Ser. No. 63/214,216 filed Jun. 23, 2021; and U.S. Ser. No. 63/214,219 filed Jun. 23, 2021, the disclosure of each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 14, 2022, is named JBI6584USNP1SEQUNCELISTING (002).txt and is 120,099 bytes in size.

### FIELD

Provided herein are binding molecules, compositions comprising same, and methods for uses thereof.

### SUMMARY

In one aspect, provided herein is an antibody that binds a CD8α hinge region, wherein the antibody is capable of binding to a functional exogenous receptor, and wherein the functional exogenous receptor comprises an extracellular domain, the CD8α hinge region, a transmembrane domain, and an intracellular signaling domain.

In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR. In some embodiments, the functional exogenous receptor is a TCR.

In some embodiments, the extracellular domain comprises an antigen binding domain derived from an antibody. In some embodiments, the extracellular domain comprises an antibody fragment. In some embodiments, the extracellular domain comprises a scFv. In some embodiments, the extracellular domain binds an antigen. In some embodiments, the antigen is a tumor antigen.

In some embodiments, the transmembrane domain is derived from a molecule selected from the group consisting of CD8a, CD4, CD28, CD137, CD80, CD86, CD152 and PD1. In some embodiments, the transmembrane domain is from CD8α or CD28.

In some embodiments, the intracellular signaling domain comprises a primary intracellular signaling domain of an immune effector cell. In some embodiments, the primary intracellular signaling domain is from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In some embodiments, the co-stimulatory signaling domain comprises a cytoplasmic domain of CD28 and/or a cytoplasmic domain of CD137.

In some embodiments, the CD8α hinge region is located between the C-terminus of the extracellular domain and the N-terminus of the transmembrane domain.

In some embodiments, the functional exogenous receptor further comprises a signal peptide. In some embodiments, the signal peptide is from CD8α.

In some embodiments, the antibody is capable of binding to an immune effector cell expressing the functional exogenous receptor, wherein optionally the immune effector cell does not express an endogenous CD8α, or the immune effector cell has been engineered to not express an endogenous CD8α. In some embodiments, the immune effector cell is a T cell, a natural killer (NK) cell, a NK T cell, a macrophage, a peripheral blood mononuclear cell (PBMC), a monocyte, a neutrophil, or an eosinophil. In some embodiments, the immune effector cell is a T cell and the T cell is a cytotoxic T cell, a helper T cell, a natural killer T cell, a αβ T cell, or a γδT cell; or wherein the immune effect cell is a NK cell.

In some embodiments, the CD8α hinge region comprises an amino acid sequence of SEQ ID NO: 209. In some embodiments, the CD8α hinge region comprises an amino acid sequence of SEQ ID NO: 210.

In another aspect, provided herein is an antibody that comprises:
(1) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2;
(2) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:27; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:28;
(3) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:54;
(4) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:79; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:80;
(5) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:106;
(6) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:131; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:x132;
(7) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:157; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:158; or
(8) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:183; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:184.

In some embodiments, the VH CDR1, VH CDR2, VH CDR3, VL CDR1, VL CDR2, and VL CDR3 amino acid sequences are according to the Kabat numbering system. In some embodiments, the VH CDR1, VH CDR2, VH CDR3, VL CDR1, VL CDR2, and VL CDR3 amino acid sequences are according to the Chothia numbering system. In some embodiments, the VH CDR1, VH CDR2, VH CDR3, VL CDR1, VL CDR2, and VL CDR3 amino acid sequences are according to the AbM numbering system. In some embodiments, the VH CDR1, VH CDR2, VH CDR3, VL CDR1, VL CDR2, and VL CDR3 amino acid sequences are according to the Contact numbering system. In some embodiments, the VH CDR1, VH CDR2, VH CDR3, VL CDR1, VL CDR2, and VL CDR3 amino acid sequences are according to the IMGT numbering system.

In some embodiments, in the antibody provided herein:

(i) the VH CDR1 comprises the amino acid sequence of SEQ ID NO:3, 9, 15, or 21, the VH CDR2 comprises the amino acid sequence of SEQ ID NO:4, 10, 16, or 22, the VH CDR3 comprises the amino acid sequence of SEQ ID NO:5, 11, 17, or 23, the VL CDR1 comprises the amino acid sequence of SEQ ID NO:6, 12, 18 or 24, the VL CDR2 comprises the amino acid sequence of SEQ ID NO:7, 13, 19, or 25, and the VL CDR3 comprises the amino acid sequence of SEQ ID NO:8, 14, 20, or 26;
(ii) the VH CDR1 comprises the amino acid sequence of SEQ ID NO:29, 35, 41, or 47, the VH CDR2 comprises the amino acid sequence of SEQ ID NO:30, 36, 42, or 48, the VH CDR3 comprises the amino acid sequence of SEQ ID NO:31, 37, 43, or 49, the VL CDR1 comprises the amino acid sequence of SEQ ID NO:32, 38, 44, or 50, the VL CDR2 comprises the amino acid sequence of SEQ ID NO:33, 39, 45, or 51, and the VL CDR3 comprises the amino acid sequence of SEQ ID NO:34, 40, 46, or 52;
(iii) the VH CDR1 comprises the amino acid sequence of SEQ ID NO:55, 61, 67, or 73, the VH CDR2 comprises the amino acid sequence of SEQ ID NO:56, 62, 68, or 74, the VH CDR3 comprises the amino acid sequence of SEQ ID NO:57, 63, 69, or 75, the VL CDR1 comprises the amino acid sequence of SEQ ID NO:58, 64, 70, or 76, the VL CDR2 comprises the amino acid sequence of SEQ ID NO:59, 65, 71, or 77, and the VL CDR3 comprises the amino acid sequence of SEQ ID NO:60, 66, 72, or 78;
(iv) the VH CDR1 comprises the amino acid sequence of SEQ ID NO:81, 87, 93, or 99, the VH CDR2 comprises the amino acid sequence of SEQ ID NO:82, 88, 94, or 100, the VH CDR3 comprises the amino acid sequence of SEQ ID NO:83, 89, 95, or 101, the VL CDR1 comprises the amino acid sequence of SEQ ID NO:84, 90, 96, or 102, the VL CDR2 comprises the amino acid sequence of SEQ ID NO:85, 91, 97, or 103, and the VL CDR3 comprises the amino acid sequence of SEQ ID NO:86, 92, 98, or 104;
(v) the VH CDR1 comprises the amino acid sequence of SEQ ID NO:107, 113, 119, or 125, the VH CDR2 comprises the amino acid sequence of SEQ ID NO:108, 114, 120, or 126, the VH CDR3 comprises the amino acid sequence of SEQ ID NO:109, 115, 121, or 127, the VL CDR1 comprises the amino acid sequence of SEQ ID NO:110, 116, 122, or 128, the VL CDR2 comprises the amino acid sequence of SEQ ID NO:111, 117, 123, or 129, and the VL CDR3 comprises the amino acid sequence of SEQ ID NO:112, 118, 124, or 130;
(vi) the VH CDR1 comprises the amino acid sequence of SEQ ID NO:133, 139, 145, or 151, the VH CDR2 comprises the amino acid sequence of SEQ ID NO:134, 140, 146, or 152, the VH CDR3 comprises the amino acid sequence of SEQ ID NO:135, 141, 147, or 153, the VL CDR1 comprises the amino acid sequence of SEQ ID NO:136, 142, 148, or 154, the VL CDR2 comprises the amino acid sequence of SEQ ID NO:137, 143, 149, or 155, and the VL CDR3 comprises the amino acid sequence of SEQ ID NO:138, 144, 150, or 156;
(vii) the VH CDR1 comprises the amino acid sequence of SEQ ID NO:159, 165, 171, or 177, the VH CDR2 comprises the amino acid sequence of SEQ ID NO:160, 166, 172, or 178, the VH CDR3 comprises the amino acid sequence of SEQ ID NO:161, 167, 173, or 179, the VL CDR1 comprises the amino acid sequence of SEQ ID NO:162, 168, 174, or 180, the VL CDR2 comprises the amino acid sequence of SEQ ID NO:163, 169, 175, or 181, and the VL CDR3 comprises the amino acid sequence of SEQ ID NO:164, 170, 176, or 182;
(viii) the VH CDR1 comprises the amino acid sequence of SEQ ID NO:185, 191, 197, or 203, the VH CDR2 comprises the amino acid sequence of SEQ ID NO:186, 192, 198, or 204, the VH CDR3 comprises the amino acid sequence of SEQ ID NO:187, 193, 199, or 205, the VL CDR1 comprises the amino acid sequence of SEQ ID NO:188, 194, 200, or 206, the VL CDR2 comprises the amino acid sequence of SEQ ID NO:189, 195, 201, or 207, and the VL CDR3 comprises the amino acid sequence of SEQ ID NO:190, 196, 202, or 208.

In some embodiments, the antibody provided herein comprises:

(i) a VH comprising the amino acid sequence of SEQ ID NO: 1, and a VL comprising the amino acid sequence of SEQ ID NO: 2;
(ii) a VH comprising the amino acid sequence of SEQ ID NO: 27, and a VL comprising the amino acid sequence of SEQ ID NO: 28;
(iii) a VH comprising the amino acid sequence of SEQ ID NO: 53, and a VL comprising the amino acid sequence of SEQ ID NO: 54;
(iv) a VH comprising the amino acid sequence of SEQ ID NO:79, and a VL comprising the amino acid sequence of SEQ ID NO:80;
(v) a VH comprising the amino acid sequence of SEQ ID NO: 105, and a VL comprising the amino acid sequence of SEQ ID NO: 106;
(vi) a VH comprising the amino acid sequence of SEQ ID NO: 131, and a VL comprising the amino acid sequence of SEQ ID NO: 132;
(vii) a VH comprising the amino acid sequence of SEQ ID NO: 157, and a VL comprising the amino acid sequence of SEQ ID NO:158; or
(viii) a VH comprising the amino acid sequence of SEQ ID NO:183, and a VL comprising the amino acid sequence of SEQ ID NO:184.

In some embodiments, the antibody is a humanized antibody. In some embodiments, the antibody is a human antibody. In some embodiments, the antibody is an IgG antibody. In some embodiments, the IgG antibody is an IgG1, IgG2, IgG3, or IgG4 antibody. In some embodiments, the antibody comprises a kappa light chain. In some embodiments, the antibody comprises a lambda light chain. In some embodiments, the antibody is a monoclonal antibody. In some embodiments, the antibody is a multivalent antibody. In some embodiments, the antibody is a multispecific antibody. In some embodiments, the antibody is genetically fused to or chemically conjugated to an agent.

In another aspect, provided herein is a nucleic acid encoding the antibody provided herein.

In another aspect, provided herein is a vector comprising the nucleic acid provided herein.

In another aspect, provided herein is a host cell comprising the vector provided herein.

In another aspect, provided herein is a kit comprising the antibody provided herein.

In another aspect, provided herein is a method for detecting and/or enriching an agent comprising a CD8α hinge region in a system comprising contacting the system with an antibody that binds a CD8α hinge region.

In another aspect, provided herein is a method for detecting and/or enriching a functional exogenous receptor in a system comprising contacting the system with an antibody that binds a CD8α hinge region, wherein the functional exogenous receptor comprises an extracellular domain, the CD8α hinge region, a transmembrane domain, and an intracellular signaling domain, wherein optionally the functional exogenous receptor is expressed in an immune effector cell.

In another aspect, provided herein is a method for detecting and/or enriching immune effector cells expressing a functional exogenous receptor comprising contacting a population of cells with an antibody that binds a CD8α hinge region, wherein the functional exogenous receptor comprises an extracellular domain, the CD8α hinge region, a transmembrane domain, and an intracellular signaling domain.

In some embodiments, the functional exogenous receptor is a T cell receptor (TCR), a chimeric antigen receptor (CAR), a chimeric TCR (cTCR), or a T cell antigen coupler (TAC)-like chimeric receptor.

In some embodiments of various methods provided herein, the antibody that binds the CD8α hinge region is the antibody provided herein.

In another aspect, provided herein is a system comprising a means for binding a CD8α hinge region in a functional exogenous receptor, wherein the functional exogenous receptor comprises an extracellular domain, the CD8α hinge region, a transmembrane domain, and an intracellular signaling domain.

In another aspect, provided herein is a system comprising a means for binding a CD8α hinge region in a functional exogenous receptor expressed in an immune effector cell, wherein the functional exogenous receptor comprises an extracellular domain, the CD8α hinge region, a transmembrane domain, and an intracellular signaling domain.

In another aspect, provided herein is a system comprising a means for binding an immune effector cell expressing a functional exogenous receptor, wherein the functional exogenous receptor comprises an extracellular domain, a CD8α hinge region, a transmembrane domain, and an intracellular signaling domain.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows a schematic of second-generation CAR-T design. A typical CAR-T molecule is composed of an antigen-binding domain scFv, a CD8α hinge as spacer between scFv transmembrane domain. CAR extracellular domain (CAR ECD) consists of scFv and CD8α hinge.

FIGS. 2A-2F show dose-dependent binding of anti-CD8α hinge mAbs to Jurkat CAR-T cells. FIGS. 2A-2B shows representative binding curves to cells expressing CD8α hinge for CD8B612 and CD8B575. FIGS. 2C-2D shows representative binding curves to cells expressing CD8α hinge for CD8B607 and CD8B611. FIGS. 2E-2F shows representative binding curves to cells expressing CD8α hinge for CD8B622 and CD8B623.

FIG. 3 shows application of the CD8α hinge binders for Jurkat CAR-T detection. The percentage of CD79b CAR+Jurkat cells (% positive) are shown in FIG. 3 using the untransduced cells as negative controls. CD8B575 equivalent showed comparable CAR-T detection sensitivity as antigen and protein L.

FIG. 4 shows application of the CD8α hinge binders for NK CAR detection. The percentage of CD79b CAR+NK cells (% positive) are shown for each cell line.

FIG. 5 shows application of the CD8α hinge binders for NK CAR detection. The percentage of CD79b CAR+NK cells (% positive) are shown for each cell line.

FIGS. 6A-6B shows binding of CD8α hinge binders to soluble CAR extracellular domain. FIG. 6A shows Bio-layer interferometry (BLI) analysis data for CD8B575. FIG. 6B shows Bio-layer interferometry (BLI) analysis data for CD8B612.

FIGS. 7A-7C show ADA assay development for select CD8α hinge binders. As shown in FIG. 7A, the anti-CD8α hinge antibody was tested in an ADA assay using a bridging assay format based on MSD™ technology platform. FIG. 7B shows the mouse version of CD8B575 and human version anti-CD8 hinge antibodies in the bridging format ADA assay. FIG. 7C shows the specificity of anti-CD8 hinge antibody, CD8B575, to CD8 hinge in CAR ECD was evaluated in a competition assay.

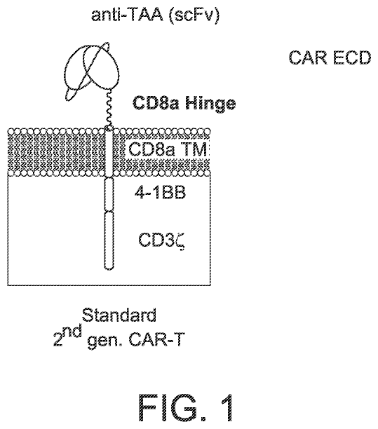

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

### 5.1. Definitions

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule disclosed herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multi specific antibodies (e.g., bispecific antibodies so long as they exhibit the desired biological activity), formed from at least two intact antibodies, single chain antibodies, single domain antiboides (e.g., VHH) and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)2 fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

“Antigen binding domain” or “antigen binding fragment” or “domain that binds an antigen” refers to a portion of a molecule that specifically binds an antigen. Antigen binding domain may include portions of an immunoglobulin that bind an antigen, such as a VH, a VL, the VH and the VL, Fab, Fab′, F(ab′)2, Fd and Fv fragments, domain antibodies (dAb) consisting of one VH or one VL, shark variable IgNAR domains, camelized VH domains, VHH, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3 and non-antibody scaffolds that bind an antigen.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, fromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a functional exogenous receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by FACS analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (KD) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat Numbering) | | | | |
| CDR H1 | H31--H35 | H26--H35 | H26--H32 | H30--H35 | |
| | (Chothia Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single chain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The term “functional exogenous receptor” as used herein, refers to an exogenous receptor (e.g., TCR such as a recombinant or engineered TCR, cTCR, TAC-like chimeric receptor, or CAR) that retains its biological activity after being introduced into an immune effector cell such as a T cell. The biological activity include but are not limited to the ability of the exogenous receptor in specifically binding to a molecule, properly transducing downstream signals, such as inducing cellular proliferation, cytokine production and/or performance of regulatory or cytolytic effector functions.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” A chimeric molecule that includes one or more antigen-binding portion (such as a single domain antibody or scFv) and a signaling domain, such as a signaling domain from a T cell receptor (e.g., CD3). Typically, CARs are comprised of an antigen-binding moiety, a transmembrane domain and an intracellular domain. The intracellular domain typically includes a signaling chain having an immunoreceptor tyrosine-based activation motif (ITAM), such as CD3 or FcεRIγ. In some instances, the intracellular domain further includes the intracellular portion of at least one additional co-stimulatory domain, such as CD28, 4-1BB (CD137), ICOS, OX40 (CD134), CD27, and/or hematopoietic cell signal transducer (DAP10). In the context of the present application, the terms “cytoplasmic domain”, “intracellular domain” and “intracellular signaling domain” are interchangeable. In some embodiments, the CAR comprises an extracellular antigen binding domain specific for one or more antigens (such as tumor antigens), a transmembrane domain, and an intracellular signaling domain of a T cell and/or other receptors. A CAR can be a single CAR, dual CAR, tandem CAR, or splict CAR. “CAR-T cell” refers to a T cell that expresses a CAR.

The term “recombinant or engineered TCR” as used herein is included as a kind of functional exogenous receptor provided herein, and refers to peptide expressed into an immune cell. The functions of recombinant or engineered TCR may include for example redirecting immune activity of the immune cell against a desired type of cells, such as cancer and infected cells having specific markers at their surface. It can replace or be-co-expressed with the endogenous TCR. In some embodiments, such recombinant TCR are single-chain TCRs comprising an open reading frame where the variable Va and VP domains are paired with a protein linker. This involves the molecular cloning of the TCR genes known to be specific for an antigen of choice. These chains are then introduced into T cells usually by means of a retroviral vector. Consequently, expression of the cloned TCRα and TCRβ genes endows the transduced T cell with a functional specificity determined by the pairing of these new genes. A component of a recombinant or engineered TCR is any functional subunit of a TCR, such as a recombined TCRα and TCRβ, which is encoded by an exogenous polynucleotide sequence introduced into the cell.

In some embodiments, the functional exogenous receptor provided herein is a chimeric TCR (cTCR), which has both antigen-binding and T-cell activating functions. For example, a cTCR can comprise: (a) an extracellular ligand binding domain comprising an antigen-binding fragment (e.g., sdAb, scFv) that specifically recognizes one or more epitopes of a tumor antigen (e.g., GPC3); (b) an optional linker; (c) an optional extracellular domain of a first TCR subunit (e.g., CD3ε) or a portion thereof; (d) a transmembrane domain comprising a transmembrane domain of a second TCR subunit (e.g., CDR); and (e) an intracellular signaling domain comprising an intracellular signaling domain of a third TCR subunit (e.g., CDR); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CDR). In some embodiments, the first, second, and third TCR subunits are different. In some embodiments, the cTCR further comprises a hinge domain located between the C-terminus of the extracellular ligand binding domain and the N-terminus of the transmembrane domain. In some embodiments, the hinge domain is derived from CD8α. In some embodiments, the cTCR further comprises a signal peptide located at the N-terminus of the cTCR, such as a signal peptide derived from CD8α.

In some embodiments, the functional exogenous receptor is a T cell antigen coupler (TAC), e.g., comprising: (a) an extracellular ligand binding domain comprising an antigen-binding fragment (e.g., sdAb, scFv) that specifically recognizes one or more epitopes of a tumor antigen (e.g., GPC3); (b) an optional first linker; (c) an extracellular TCR binding domain that specifically recognizes the extracellular domain of a TCR subunit (e.g., CDR); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain.

In some embodiments, the functional exogenous receptor is a TAC-like chimeric receptor, e.g., comprising: (a) an extracellular ligand binding domain comprising an antigen-binding fragment (e.g., sdAb, scFv) that specifically recognizes one or more epitopes of a tumor antigen (e.g., GPC3); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CDR); and (g) an optional intracellular signaling domain comprising an intracellular signaling domain of a fourth TCR subunit (e.g., CDR); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage”, “managing” and “management” refer to the beneficial effects that a subject derives from a therapy which does not necessarily result in a cure of the disease.

The terms “prevent”, “preventing” and “prevention” refer to reducing the likelihood of the onset (or recurrence) of a disease, disorder, condition, or associated symptom(s) (e.g., a cancer).

As used herein, the term “CD8” refers to CD8 from any species, such as from primate or rodent, such as human, monkey, rat or mouse. Human CD8 is a homodimer of alpha chains (CD8α) or a heterodimer of CD8α (SEQ ID NO: 211) and CD8β (SEQ ID NO: 212) chains.

(CD8α chain)
SEQ ID NO: 211
SQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALS
NSIMYFSHFVPVFLPAKPTTTPAPRPPTPAPTIASQPLSLRPEACRPAA
GGAVHTRGLDFACDIYIWAPLAGTCGVLLLSLVITLYCNHRNRRRVCKC
PRPVVKSGDKPSLSARYV
(CD8β chain)
SEQ ID NO: 212
LQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIV
GSPELTFGKGTQLSVVDFLPTTAQPTKKSTLKKRVCRLPRPETQKGPLC
SPITLGLLVAGVLVLLVSLGVAIHLCCRRRRARLRFMKQFYK

### 5.2 Antibodies

Provided herein are anti-CD8α hinge antibodies or antigen-binding fragments thereof, nucleic acids and expression vectors encoding the antibodies, recombinant cells containing the vectors, and compositions comprising the antibodies. Methods of making the antibodies, and methods of using the antibodies are also provided. The antibodies disclosed herein possess one or more desirable functional properties, including but not limited to high-affinity binding to CD8α hinge region or high specificity to CD8α hinge region.

As used herein, the term “CD8α hinge” refers to the hinge region of CD8α. The term “CD8α hinge” as used herein includes the hinge region referred to in Section 5.3.6. The term “antibody against CD8α hinge” or “anti-CD8α hinge antibody” as used herein relates to an antibody specifically binding to CD8α hinge. In some embodiments, the CD8α hinge region comprises an amino acid sequence of TTPAPRPPTPAPTIASQPLSLRPEACRPAAGGAVHTRGLDFACD (SEQ ID NO: 209). In some embodiments, the CD8α hinge region comprises an amino acid sequence of STPAPRPPTPAPTIASQPLSLRPEACRPAAGGAVHTRGLDFACD (SEQ ID NO: 210).

As used herein, an antibody that “specifically binds to CD8α hinge” refers to an antibody that binds to a CD8α hinge, preferably a human CD8α hinge, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

In one aspect, provided herein is an antibody that binds to CD8α hinge. In some embodiments, the antibody comprises a heavy chain variable region and a light chain variable region. In some embodiments, the CD8α hinge antibody is not a single domain antibody or nanobody. In some embodiments, the CD8α hinge antibody is a humanized antibody.

In certain embodiments, provided herein is an anti-CD8α hinge antibody comprising a VH region, VL region, VH CDR1, VH CDR2, VH CDR3, VL CDR1, VL CDR2, and/or VL CDR3 of any one of the antibodies described herein. In some embodiments, provided herein is an anti-CD8α hinge antibody comprising a VH region of any one of the antibodies described herein. In some embodiments, provided herein is an anti-CD8α hinge antibody comprising a VL region of any one of the antibodies described herein. In some embodiments, provided herein is an anti-CD8α hinge antibody comprising a VH region of any one of the antibodies described herein, and a VL region of any one of the antibodies described herein. In some embodiments, provided herein is an anti-CD8α hinge antibody comprising a VH CDR1, VH CDR2, and VH CDR3 of any one of the antibodies described herein. In some embodiments, provided herein is an anti-CD8α hinge antibody comprising a VL CDR1, VL CDR2, and VL CDR3 of any one of the antibodies described herein. In some embodiments, provided herein is an anti-CD8α hinge antibody comprising a VH CDR1, VH CDR2, and VH CDR3 of any one of the antibodies described herein; and a VL CDR1, VL CDR2, and VL CDR3 of any one of the antibodies described herein.

In certain embodiments, provided is an anti-CD8α hinge antibody that is an intact antibody. In other embodiments, provided is an anti-CD8α hinge antibody is an antigen binding fragment of the anti-CD8α hinge antibody. In some embodiments, the antigen binding fragment of the anti-CD8α hinge antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the anti-CD8α hinge antibody comprises a VH region and a VL region.

In some embodiments, the anti-CD8α hinge antibody is a single chain antibody. In some embodiments, the anti-CD8α hinge antibody is a single domain antibody. In some embodiments, the anti-CD8α hinge antibody is a nanobody. In certain embodiments, the anti-CD8α hinge antibody is a VHH antibody. In some embodiments, the anti-CD8α hinge antibody is not a single chain antibody. In some embodiments, the anti-CD8α hinge antibody is not a single domain antibody. In some embodiments, the anti-CD8α hinge antibody is not a nanobody. In certain embodiments, the anti-CD8α hinge antibody is not a VHH antibody.

In some embodiments, the anti-CD8α hinge antibody is a multispecific antibody. In other embodiments, the anti-CD8α hinge is a bispecific antibody. In certain embodiments, the multispecific antibody comprises an antigen binding fragment of an anti-CD8α hinge antibody provided herein. In other embodiments, the bispecific antibody comprises an antigen binding fragment of an anti-CD8α hinge antibody provided herein. In a specific embodiment, a CD8α hinge antibody, or antigen binding fragment thereof, provided herein specifically binds to CD8α hinge.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In another aspect, provided herein is an antibody that binds CD8α hinge, comprising: (i) a VH comprising a VH CDR1 having an amino acid sequence of SEQ ID NO:3, a VH CDR2 having an amino acid sequence of SEQ ID NO:4, and a VH CDR3 having an amino acid sequence of SEQ ID NO:5; and (ii) a VL comprising a VL CDR1 having an amino acid sequence of SEQ ID NO:6, a VL CDR2 having an amino acid sequence of SEQ ID NO:7, and a VL CDR3 having an amino acid sequence of SEQ ID NO:8. In another aspect, provided herein is an antibody that binds CD8α hinge, comprising: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11; and (ii) a VL comprising a VL CDR1 having an amino acid sequence of SEQ ID NO12, a VL CDR2 having an amino acid sequence of SEQ ID NO:13, and a VL CDR3 having an amino acid sequence of SEQ ID NO:14. In another aspect, provided herein is an antibody that binds CD8α hinge, comprising: (i) a VH comprising a VH CDR1 having an amino acid sequence of SEQ ID NO:15, a VH CDR2 having an amino acid sequence of SEQ ID NO:16, and a VH CDR3 having an amino acid sequence of SEQ ID NO:17; and (ii) a VL comprising a VL CDR1 having an amino acid sequence of SEQ ID NO:18, a VL CDR2 having an amino acid sequence of SEQ ID NO:19, and a VL CDR3 having an amino acid sequence of SEQ ID NO:20. In another aspect, provided herein is an antibody that binds CD8α hinge, comprising: (i) a VH comprising a VH CDR1 having an amino acid sequence of SEQ ID NO:21, a VH CDR2 having an amino acid sequence of SEQ ID NO:22, and a VH CDR3 having an amino acid sequence of SEQ ID NO:23; and (ii) a VL comprising a VL CDR1 having an amino acid sequence of SEQ ID NO:24, a VL CDR2 having an amino acid sequence of SEQ ID NO:25, and a VL CDR3 having an amino acid sequence of SEQ ID NO:26.

In another aspect, provided herein is an antibody that binds CD8α hinge, comprising: (i) a VH comprising a VH CDR1 having an amino acid sequence of SEQ ID NO:29, a VH CDR2 having an amino acid sequence of SEQ ID NO:30, and a VH CDR3 having an amino acid sequence of SEQ ID NO:31; and (ii) a VL comprising a VL CDR1 having an amino acid sequence of SEQ ID NO:32, a VL CDR2 having an amino acid sequence of SEQ ID NO:33, and a VL CDR3 having an amino acid sequence of SEQ ID NO:34. In another aspect, provided herein is an antibody that binds CD8α hinge, comprising: (i) a VH comprising a VH CDR1 having an amino acid sequence of SEQ ID NO:35, a VH CDR2 having an amino acid sequence of SEQ ID NO:36, and a VH CDR3 having an amino acid sequence of SEQ ID NO:37; and (ii) a VL comprising a VL CDR1 having an amino acid sequence of SEQ ID NO:38, a VL CDR2 having an amino acid sequence of SEQ ID NO:39, and a VL CDR3 having an amino acid sequence of SEQ ID NO:40. In another aspect, provided herein is an antibody that binds CD8α hinge, comprising: (i) a VH comprising a VH CDR1 having an amino acid sequence of SEQ ID NO:41, a VH CDR2 having an amino acid sequence of SEQ ID NO:42, and a VH CDR3 having an amino acid sequence of SEQ ID NO:43; and (ii) a VL comprising a VL CDR1 having an amino acid sequence of SEQ ID NO:44, a VL CDR2 having an amino acid sequence of SEQ ID NO:45, and a VL CDR3 having an amino acid sequence of SEQ ID NO:46. In another aspect, provided herein is an antibody that binds CD8α hinge, comprising: (i) a VH comprising a VH CDR1 having an amino acid sequence of SEQ ID NO:47, a VH CDR2 having an amino acid sequence of SEQ ID NO:48, and a VH CDR3 having an amino acid sequence of SEQ ID NO:49; and (ii) a VL comprising a VL CDR1 having an amino acid sequence of SEQ ID NO:50, a VL CDR2 having an amino acid sequence of SEQ ID NO:51, and a VL CDR3 having an amino acid sequence of SEQ ID NO:52.

In another aspect, provided herein is an antibody that binds CD8α hinge, comprising: (i) a VH comprising a VH CDR1 having an amino acid sequence of SEQ ID NO:55, a VH CDR2 having an amino acid sequence of SEQ ID NO:56, and a VH CDR3 having an amino acid sequence of SEQ ID NO:57; and (ii) a VL comprising a VL CDR1 having an amino acid sequence of SEQ ID NO:58, a VL CDR2 having an amino acid sequence of SEQ ID NO:59, and a VL CDR3 having an amino acid sequence of SEQ ID NO:60. In another aspect, provided herein is an antibody that binds CD8α hinge, comprising: (i) a VH comprising a VH CDR1 having an amino acid sequence of SEQ ID NO:61, a VH CDR2 having an amino acid sequence of SEQ ID NO:62, and a VH CDR3 having an amino acid sequence of SEQ ID NO:63; and (ii) a VL comprising a VL CDR1 having an amino acid sequence of SEQ ID NO:64, a VL CDR2 having an amino acid sequence of SEQ ID NO:65, and a VL CDR3 having an amino acid sequence of SEQ ID NO:66. In another aspect, provided herein is an antibody that binds CD8α hinge, comprising: (i) a VH comprising a VH CDR1 having an amino acid sequence of SEQ ID NO:67, a VH CDR2 having an amino acid sequence of SEQ ID NO:68, and a VH CDR3 having an amino acid sequence of SEQ ID NO:69; and (ii) a VL comprising a VL CDR1 having an amino acid sequence of SEQ ID NO:70, a VL CDR2 having an amino acid sequence of SEQ ID NO:71, and a VL CDR3 having an amino acid sequence of SEQ ID NO:72. In another aspect, provided herein is an antibody that binds CD8α hinge, comprising: (i) a VH comprising a VH CDR1 having an amino acid sequence of SEQ ID NO:73, a VH CDR2 having an amino acid sequence of SEQ ID NO:74, and a VH CDR3 having an amino acid sequence of SEQ ID NO:75; and (ii) a VL comprising a VL CDR1 having an amino acid sequence of SEQ ID NO:76, a VL CDR2 having an amino acid sequence of SEQ ID NO:77, and a VL CDR3 having an amino acid sequence of SEQ ID NO:78.

In another aspect, provided herein is an antibody that binds CD8α hinge, comprising: (i) a VH comprising a VH CDR1 having an amino acid sequence of SEQ ID NO:81, a VH CDR2 having an amino acid sequence of SEQ ID NO:82, and a VH CDR3 having an amino acid sequence of SEQ ID NO:83; and (ii) a VL comprising a VL CDR1 having an amino acid sequence of SEQ ID NO:84, a VL CDR2 having an amino acid sequence of SEQ ID NO:85, and a VL CDR3 having an amino acid sequence of SEQ ID NO:86. In another aspect, provided herein is an antibody that binds CD8α hinge, comprising: (i) a VH comprising a VH CDR1 having an amino acid sequence of SEQ ID NO:87, a VH CDR2 having an amino acid sequence of SEQ ID NO:88, and a VH CDR3 having an amino acid sequence of SEQ ID NO:89; and (ii) a VL comprising a VL CDR1 having an amino acid sequence of SEQ ID NO:90, a VL CDR2 having an amino acid sequence of SEQ ID NO:91, and a VL CDR3 having an amino acid sequence of SEQ ID NO:92. In another aspect, provided herein is an antibody that binds CD8α hinge, comprising: (i) a VH comprising a VH CDR1 having an amino acid sequence of SEQ ID NO:93, a VH CDR2 having an amino acid sequence of SEQ ID NO:94, and a VH CDR3 having an amino acid sequence of SEQ ID NO:95; and (ii) a VL comprising a VL CDR1 having an amino acid sequence of SEQ ID NO:96, a VL CDR2 having an amino acid sequence of SEQ ID NO:97, and a VL CDR3 having an amino acid sequence of SEQ ID NO:98. In another aspect, provided herein is an antibody that binds CD8α hinge, comprising: (i) a VH comprising a VH CDR1 having an amino acid sequence of SEQ ID NO:99, a VH CDR2 having an amino acid sequence of SEQ ID NO:100, and a VH CDR3 having an amino acid sequence of SEQ ID NO:101; and (ii) a VL comprising a VL CDR1 having an amino acid sequence of SEQ ID NO:102, a VL CDR2 having an amino acid sequence of SEQ ID NO:103, and a VL CDR3 having an amino acid sequence of SEQ ID NO:104.

In another aspect, provided herein is an antibody that binds CD8α hinge, comprising: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In another aspect, provided herein is an antibody that binds CD8α hinge, comprising: (i) a VH comprising a VH CDR1 having an amino acid sequence of SEQ ID NO:113, a VH CDR2 having an amino acid sequence of SEQ ID NO:114, and a VH CDR3 having an amino acid sequence of SEQ ID NO:115; and (ii) a VL comprising a VL CDR1 having an amino acid sequence of SEQ ID NO:116, a VL CDR2 having an amino acid sequence of SEQ ID NO:117, and a VL CDR3 having an amino acid sequence of SEQ ID NO:118. In another aspect, provided herein is an antibody that binds CD8α hinge, comprising: (i) a VH comprising a VH CDR1 having an amino acid sequence of SEQ ID NO:119, a VH CDR2 having an amino acid sequence of SEQ ID NO:120, and a VH CDR3 having an amino acid sequence of SEQ ID NO:121; and (ii) a VL comprising a VL CDR1 having an amino acid sequence of SEQ ID NO:122, a VL CDR2 having an amino acid sequence of SEQ ID NO:123, and a VL CDR3 having an amino acid sequence of SEQ ID NO:124. In another aspect, provided herein is an antibody that binds CD8α hinge, comprising: (i) a VH comprising a VH CDR1 having an amino acid sequence of SEQ ID NO:125, a VH CDR2 having an amino acid sequence of SEQ ID NO:126, and a VH CDR3 having an amino acid sequence of SEQ ID NO:127; and (ii) a VL comprising a VL CDR1 having an amino acid sequence of SEQ ID NO:128, a VL CDR2 having an amino acid sequence of SEQ ID NO:129, and a VL CDR3 having an amino acid sequence of SEQ ID NO:130.

In another aspect, provided herein is an antibody that binds CD8α hinge, comprising: (i) a VH comprising a VH CDR1 having an amino acid sequence of SEQ ID NO:133, a VH CDR2 having an amino acid sequence of SEQ ID NO:134, and a VH CDR3 having an amino acid sequence of SEQ ID NO:135; and (ii) a VL comprising a VL CDR1 having an amino acid sequence of SEQ ID NO:136, a VL CDR2 having an amino acid sequence of SEQ ID NO:137, and a VL CDR3 having an amino acid sequence of SEQ ID NO:138. In another aspect, provided herein is an antibody that binds CD8α hinge, comprising: (i) a VH comprising a VH CDR1 having an amino acid sequence of SEQ ID NO:139, a VH CDR2 having an amino acid sequence of SEQ ID NO:140, and a VH CDR3 having an amino acid sequence of SEQ ID NO:141; and (ii) a VL comprising a VL CDR1 having an amino acid sequence of SEQ ID NO:142, a VL CDR2 having an amino acid sequence of SEQ ID NO:143, and a VL CDR3 having an amino acid sequence of SEQ ID NO:144. In another aspect, provided herein is an antibody that binds CD8α hinge, comprising: (i) a VH comprising a VH CDR1 having an amino acid sequence of SEQ ID NO:145, a VH CDR2 having an amino acid sequence of SEQ ID NO:146, and a VH CDR3 having an amino acid sequence of SEQ ID NO:147; and (ii) a VL comprising a VL CDR1 having an amino acid sequence of SEQ ID NO:148, a VL CDR2 having an amino acid sequence of SEQ ID NO:149, and a VL CDR3 having an amino acid sequence of SEQ ID NO:150. In another aspect, provided herein is an antibody that binds CD8α hinge, comprising: (i) a VH comprising a VH CDR1 having an amino acid sequence of SEQ ID NO:151, a VH CDR2 having an amino acid sequence of SEQ ID NO:152, and a VH CDR3 having an amino acid sequence of SEQ ID NO:153; and (ii) a VL comprising a VL CDR1 having an amino acid sequence of SEQ ID NO:154, a VL CDR2 having an amino acid sequence of SEQ ID NO:155, and a VL CDR3 having an amino acid sequence of SEQ ID NO:156.

In another aspect, provided herein is an antibody that binds CD8α hinge, comprising: (i) a VH comprising a VH CDR1 having an amino acid sequence of SEQ ID NO:159, a VH CDR2 having an amino acid sequence of SEQ ID NO:160, and a VH CDR3 having an amino acid sequence of SEQ ID NO:161; and (ii) a VL comprising a VL CDR1 having an amino acid sequence of SEQ ID NO:162, a VL CDR2 having an amino acid sequence of SEQ ID NO:163, and a VL CDR3 having an amino acid sequence of SEQ ID NO:164. In another aspect, provided herein is an antibody that binds CD8α hinge, comprising: (i) a VH comprising a VH CDR1 having an amino acid sequence of SEQ ID NO:165, a VH CDR2 having an amino acid sequence of SEQ ID NO:166, and a VH CDR3 having an amino acid sequence of SEQ ID NO:167; and (ii) a VL comprising a VL CDR1 having an amino acid sequence of SEQ ID NO:168, a VL CDR2 having an amino acid sequence of SEQ ID NO:169, and a VL CDR3 having an amino acid sequence of SEQ ID NO:170. In another aspect, provided herein is an antibody that binds CD8α hinge, comprising: (i) a VH comprising a VH CDR1 having an amino acid sequence of SEQ ID NO:171, a VH CDR2 having an amino acid sequence of SEQ ID NO:172, and a VH CDR3 having an amino acid sequence of SEQ ID NO:173; and (ii) a VL comprising a VL CDR1 having an amino acid sequence of SEQ ID NO:174, a VL CDR2 having an amino acid sequence of SEQ ID NO:175, and a VL CDR3 having an amino acid sequence of SEQ ID NO:176. In another aspect, provided herein is an antibody that binds CD8α hinge, comprising: (i) a VH comprising a VH CDR1 having an amino acid sequence of SEQ ID NO:177, a VH CDR2 having an amino acid sequence of SEQ ID NO:178, and a VH CDR3 having an amino acid sequence of SEQ ID NO:179; and (ii) a VL comprising a VL CDR1 having an amino acid sequence of SEQ ID NO:180, a VL CDR2 having an amino acid sequence of SEQ ID NO:181, and a VL CDR3 having an amino acid sequence of SEQ ID NO:182.

In another aspect, provided herein is an antibody that binds CD8α hinge, comprising: (i) a VH comprising a VH CDR1 having an amino acid sequence of SEQ ID NO:185, a VH CDR2 having an amino acid sequence of SEQ ID NO:186, and a VH CDR3 having an amino acid sequence of SEQ ID NO:187; and (ii) a VL comprising a VL CDR1 having an amino acid sequence of SEQ ID NO:188, a VL CDR2 having an amino acid sequence of SEQ ID NO:189, and a VL CDR3 having an amino acid sequence of SEQ ID NO:190. In another aspect, provided herein is an antibody that binds CD8α hinge, comprising: (i) a VH comprising a VH CDR1 having an amino acid sequence of SEQ ID NO:191, a VH CDR2 having an amino acid sequence of SEQ ID NO:192, and a VH CDR3 having an amino acid sequence of SEQ ID NO:193; and (ii) a VL comprising a VL CDR1 having an amino acid sequence of SEQ ID NO:194, a VL CDR2 having an amino acid sequence of SEQ ID NO:195, and a VL CDR3 having an amino acid sequence of SEQ ID NO:196. In another aspect, provided herein is an antibody that binds CD8α hinge, comprising: (i) a VH comprising a VH CDR1 having an amino acid sequence of SEQ ID NO:197, a VH CDR2 having an amino acid sequence of SEQ ID NO:198, and a VH CDR3 having an amino acid sequence of SEQ ID NO:199; and (ii) a VL comprising a VL CDR1 having an amino acid sequence of SEQ ID NO:200, a VL CDR2 having an amino acid sequence of SEQ ID NO:201, and a VL CDR3 having an amino acid sequence of SEQ ID NO:202. In another aspect, provided herein is an antibody that binds CD8α hinge, comprising: (i) a VH comprising a VH CDR1 having an amino acid sequence of SEQ ID NO:203, a VH CDR2 having an amino acid sequence of SEQ ID NO:204, and a VH CDR3 having an amino acid sequence of SEQ ID NO:205; and (ii) a VL comprising a VL CDR1 having an amino acid sequence of SEQ ID NO:206, a VL CDR2 having an amino acid sequence of SEQ ID NO:207, and a VL CDR3 having an amino acid sequence of SEQ ID NO:208.

In some embodiments, the antibody comprises a VH having an amino acid sequence of SEQ ID NO:1. In some embodiments, the antibody comprises a VL having an amino acid sequence of SEQ ID NO:2. In some embodiments, the antibody comprises a VH having an amino acid sequence of SEQ ID NO:1, and a VL having an amino acid sequence of SEQ ID NO:2. In some embodiments, the antibody comprises a heavy chain having an amino acid sequence of SEQ ID NO:215. In some embodiments, the antibody comprises a light chain having an amino acid sequence of SEQ ID NO:216. In some embodiments, the antibody comprises a heavy chain having an amino acid sequence of SEQ ID NO:215, and a light chain having an amino acid sequence of SEQ ID NO:216. In some embodiments, the antibody comprises a VH having an amino acid sequence having at least 95% identity to an amino acid sequence of SEQ ID NO:1. In some embodiments, the antibody comprises a VL having an amino acid sequence having at least 95% identity to an amino acid sequence of SEQ ID NO:2. In some embodiments, the antibody comprises a VH having an amino acid sequence having at least 95% identity to an amino acid sequence of SEQ ID NO:1, and a VL having an amino acid sequence having at least 95% identity to an amino acid sequence of SEQ ID NO:2. In some embodiments, the antibody comprises a heavy chain having an amino acid sequence having at least 95% identity to an amino acid sequence of SEQ ID NO:215. In some embodiments, the antibody comprises a light chain having an amino acid sequence having at least 95% identity to an amino acid sequence of SEQ ID NO:216. In some embodiments, the antibody comprises a heavy chain having an amino acid sequence having at least 95% identity to an amino acid sequence of SEQ ID NO:215, and a light chain having an amino acid sequence having at least 95% identity to an amino acid sequence of SEQ ID NO:216.

In some embodiments, the antibody comprises a VH having an amino acid sequence of SEQ ID NO:27. In some embodiments, the antibody comprises a VL having an amino acid sequence of SEQ ID NO:28. In some embodiments, the antibody comprises a VH having an amino acid sequence of SEQ ID NO:27, and a VL having an amino acid sequence of SEQ ID NO:28. In some embodiments, the antibody comprises a heavy chain having an amino acid sequence of SEQ ID NO:217. In some embodiments, the antibody comprises a light chain having an amino acid sequence of SEQ ID NO:218. In some embodiments, the antibody comprises a heavy chain having an amino acid sequence of SEQ ID NO:217, and a light chain having an amino acid sequence of SEQ ID NO:218. In some embodiments, the antibody comprises a VH having an amino acid sequence having at least 95% identity to an amino acid sequence of SEQ ID NO:27. In some embodiments, the antibody comprises a VL having an amino acid sequence having at least 95% identity to an amino acid sequence of SEQ ID NO:28. In some embodiments, the antibody comprises a VH having an amino acid sequence having at least 95% identity to an amino acid sequence of SEQ ID NO:27, and a VL having an amino acid sequence having at least 95% identity to an amino acid sequence of SEQ ID NO:28. In some embodiments, the antibody comprises a heavy chain having an amino acid sequence having at least 95% identity to an amino acid sequence of SEQ ID NO:217. In some embodiments, the antibody comprises a light chain having an amino acid sequence having at least 95% identity to an amino acid sequence of SEQ ID NO:218. In some embodiments, the antibody comprises a heavy chain having an amino acid sequence having at least 95% identity to an amino acid sequence of SEQ ID NO:217, and a light chain having an amino acid sequence having at least 95% identity to an amino acid sequence of SEQ ID NO:218.

In some embodiments, the antibody comprises a VH having an amino acid sequence of SEQ ID NO:53. In some embodiments, the antibody comprises a VL having an amino acid sequence of SEQ ID NO:54. In some embodiments, the antibody comprises a VH having an amino acid sequence of SEQ ID NO:53, and a VL having an amino acid sequence of SEQ ID NO:54. In some embodiments, the antibody comprises a heavy chain having an amino acid sequence of SEQ ID NO:219. In some embodiments, the antibody comprises a light chain having an amino acid sequence of SEQ ID NO:220. In some embodiments, the antibody comprises a heavy chain having an amino acid sequence of SEQ ID NO:219, and a light chain having an amino acid sequence of SEQ ID NO:220. In some embodiments, the antibody comprises a VH having an amino acid sequence having at least 95% identity to an amino acid sequence of SEQ ID NO:53. In some embodiments, the antibody comprises a VL having an amino acid sequence having at least 95% identity to an amino acid sequence of SEQ ID NO:54. In some embodiments, the antibody comprises a VH having an amino acid sequence having at least 95% identity to an amino acid sequence of SEQ ID NO:53, and a VL having an amino acid sequence having at least 95% identity to an amino acid sequence of SEQ ID NO:54. In some embodiments, the antibody comprises a heavy chain having an amino acid sequence having at least 95% identity to an amino acid sequence of SEQ ID NO:219. In some embodiments, the antibody comprises a light chain having an amino acid sequence having at least 95% identity to an amino acid sequence of SEQ ID NO:220. In some embodiments, the antibody comprises a heavy chain having an amino acid sequence having at least 95% identity to an amino acid sequence of SEQ ID NO:219, and a light chain having an amino acid sequence having at least 95% identity to an amino acid sequence of SEQ ID NO:220.

In some embodiments, the antibody comprises a VH having an amino acid sequence of SEQ ID NO:79. In some embodiments, the antibody comprises a VL having an amino acid sequence of SEQ ID NO:80. In some embodiments, the antibody comprises a VH having an amino acid sequence of SEQ ID NO:79, and a VL having an amino acid sequence of SEQ ID NO:80. In some embodiments, the antibody comprises a heavy chain having an amino acid sequence of SEQ ID NO:221. In some embodiments, the antibody comprises a light chain having an amino acid sequence of SEQ ID NO:222. In some embodiments, the antibody comprises a heavy chain having an amino acid sequence of SEQ ID NO:221, and a light chain having an amino acid sequence of SEQ ID NO:222. In some embodiments, the antibody comprises a VH having an amino acid sequence having at least 95% identity to an amino acid sequence of SEQ ID NO:79. In some embodiments, the antibody comprises a VL having an amino acid sequence having at least 95% identity to an amino acid sequence of SEQ ID NO:80. In some embodiments, the antibody comprises a VH having an amino acid sequence having at least 95% identity to an amino acid sequence of SEQ ID NO:79, and a VL having an amino acid sequence having at least 95% identity to an amino acid sequence of SEQ ID NO:80. In some embodiments, the antibody comprises a heavy chain having an amino acid sequence having at least 95% identity to an amino acid sequence of SEQ ID NO:221. In some embodiments, the antibody comprises a light chain having an amino acid sequence having at least 95% identity to an amino acid sequence of SEQ ID NO:222. In some embodiments, the antibody comprises a heavy chain having an amino acid sequence having at least 95% identity to an amino acid sequence of SEQ ID NO:221, and a light chain having an amino acid sequence having at least 95% identity to an amino acid sequence of SEQ ID NO:222.

In some embodiments, the antibody comprises a VH having an amino acid sequence of SEQ ID NO:105. In some embodiments, the antibody comprises a VL having an amino acid sequence of SEQ ID NO:106. In some embodiments, the antibody comprises a VH having an amino acid sequence of SEQ ID NO:105, and a VL having an amino acid sequence of SEQ ID NO:106. In some embodiments, the antibody comprises a heavy chain having an amino acid sequence of SEQ ID NO:223. In some embodiments, the antibody comprises a light chain having an amino acid sequence of SEQ ID NO:224. In some embodiments, the antibody comprises a heavy chain having an amino acid sequence of SEQ ID NO:223, and a light chain having an amino acid sequence of SEQ ID NO:224. In some embodiments, the antibody comprises a VH having an amino acid sequence having at least 95% identity to an amino acid sequence of SEQ ID NO:105. In some embodiments, the antibody comprises a VL having an amino acid sequence having at least 95% identity to an amino acid sequence of SEQ ID NO:106. In some embodiments, the antibody comprises a VH having an amino acid sequence having at least 95% identity to an amino acid sequence of SEQ ID NO:105, and a VL having an amino acid sequence having at least 95% identity to an amino acid sequence of SEQ ID NO:106. In some embodiments, the antibody comprises a heavy chain having an amino acid sequence having at least 95% identity to an amino acid sequence of SEQ ID NO:223. In some embodiments, the antibody comprises a light chain having an amino acid sequence having at least 95% identity to an amino acid sequence of SEQ ID NO:224. In some embodiments, the antibody comprises a heavy chain having an amino acid sequence having at least 95% identity to an amino acid sequence of SEQ ID NO:223, and a light chain having an amino acid sequence having at least 95% identity to an amino acid sequence of SEQ ID NO:224.

In some embodiments, the antibody comprises a VH having an amino acid sequence of SEQ ID NO:131. In some embodiments, the antibody comprises a VL having an amino acid sequence of SEQ ID NO:132. In some embodiments, the antibody comprises a VH having an amino acid sequence of SEQ ID NO:131, and a VL having an amino acid sequence of SEQ ID NO:132. In some embodiments, the antibody comprises a heavy chain having an amino acid sequence of SEQ ID NO:225. In some embodiments, the antibody comprises a light chain having an amino acid sequence of SEQ ID NO:226. In some embodiments, the antibody comprises a heavy chain having an amino acid sequence of SEQ ID NO:225, and a light chain having an amino acid sequence of SEQ ID NO:226. In some embodiments, the antibody comprises a VH having an amino acid sequence having at least 95% identity to an amino acid sequence of SEQ ID NO:131. In some embodiments, the antibody comprises a VL having an amino acid sequence having at least 95% identity to an amino acid sequence of SEQ ID NO:132. In some embodiments, the antibody comprises a VH having an amino acid sequence having at least 95% identity to an amino acid sequence of SEQ ID NO:131, and a VL having an amino acid sequence having at least 95% identity to an amino acid sequence of SEQ ID NO:132. In some embodiments, the antibody comprises a heavy chain having an amino acid sequence having at least 95% identity to an amino acid sequence of SEQ ID NO:225. In some embodiments, the antibody comprises a light chain having an amino acid sequence having at least 95% identity to an amino acid sequence of SEQ ID NO:226. In some embodiments, the antibody comprises a heavy chain having an amino acid sequence having at least 95% identity to an amino acid sequence of SEQ ID NO:225, and a light chain having an amino acid sequence having at least 95% identity to an amino acid sequence of SEQ ID NO:226.

In some embodiments, the antibody comprises a VH having an amino acid sequence of SEQ ID NO:157. In some embodiments, the antibody comprises a VL having an amino acid sequence of SEQ ID NO:158. In some embodiments, the antibody comprises a VH having an amino acid sequence of SEQ ID NO:157, and a VL having an amino acid sequence of SEQ ID NO:158. In some embodiments, the antibody comprises a heavy chain having an amino acid sequence of SEQ ID NO:227. In some embodiments, the antibody comprises a light chain having an amino acid sequence of SEQ ID NO:228. In some embodiments, the antibody comprises a heavy chain having an amino acid sequence of SEQ ID NO:227, and a light chain having an amino acid sequence of SEQ ID NO:228. In some embodiments, the antibody comprises a VH having an amino acid sequence having at least 95% identity to an amino acid sequence of SEQ ID NO:157. In some embodiments, the antibody comprises a VL having an amino acid sequence having at least 95% identity to an amino acid sequence of SEQ ID NO:158. In some embodiments, the antibody comprises a VH having an amino acid sequence having at least 95% identity to an amino acid sequence of SEQ ID NO:157, and a VL having an amino acid sequence having at least 95% identity to an amino acid sequence of SEQ ID NO:158. In some embodiments, the antibody comprises a heavy chain having an amino acid sequence having at least 95% identity to an amino acid sequence of SEQ ID NO:227. In some embodiments, the antibody comprises a light chain having an amino acid sequence having at least 95% identity to an amino acid sequence of SEQ ID NO:228. In some embodiments, the antibody comprises a heavy chain having an amino acid sequence having at least 95% identity to an amino acid sequence of SEQ ID NO:227, and a light chain having an amino acid sequence having at least 95% identity to an amino acid sequence of SEQ ID NO:228.

In some embodiments, the antibody comprises a VH having an amino acid sequence of SEQ ID NO:183. In some embodiments, the antibody comprises a VL having an amino acid sequence of SEQ ID NO:184. In some embodiments, the antibody comprises a VH having an amino acid sequence of SEQ ID NO:183, and a VL having an amino acid sequence of SEQ ID NO:184. In some embodiments, the antibody comprises a heavy chain having an amino acid sequence of SEQ ID NO:229. In some embodiments, the antibody comprises a light chain having an amino acid sequence of SEQ ID NO:230. In some embodiments, the antibody comprises a heavy chain having an amino acid sequence of SEQ ID NO:229, and a light chain having an amino acid sequence of SEQ ID NO:230. In some embodiments, the antibody comprises a VH having an amino acid sequence having at least 95% identity to an amino acid sequence of SEQ ID NO:183. In some embodiments, the antibody comprises a VL having an amino acid sequence having at least 95% identity to an amino acid sequence of SEQ ID NO:184. In some embodiments, the antibody comprises a VH having an amino acid sequence having at least 95% identity to an amino acid sequence of SEQ ID NO:183, and a VL having an amino acid sequence having at least 95% identity to an amino acid sequence of SEQ ID NO:184. In some embodiments, the antibody comprises a heavy chain having an amino acid sequence having at least 95% identity to an amino acid sequence of SEQ ID NO:229. In some embodiments, the antibody comprises a light chain having an amino acid sequence having at least 95% identity to an amino acid sequence of SEQ ID NO:230. In some embodiments, the antibody comprises a heavy chain having an amino acid sequence having at least 95% identity to an amino acid sequence of SEQ ID NO:229, and a light chain having an amino acid sequence having at least 95% identity to an amino acid sequence of SEQ ID NO:230.

In some embodiments, the CD8α hinge antibody is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In some embodiments, the CD8α hinge antibody is multivalent. In some embodiments, the CD8α hinge antibody is capable of binding at least three antigens. In some embodiments, the CD8α hinge antibody is capable of binding at least five antigens.

In another aspect, provided herein is an antibody that competes for binding to CD8α hinge with any of the CD8α hinge antibodies described herein. In another aspect, provided herein is an antibody that binds to the same epitope as any of the CD8α hinge antibodies described herein. In another aspect, provided is a CD8α hinge antibody that binds an epitope on CD8α hinge that overlaps with the epitope on CD8α hinge bound by a CD8α hinge antibody described herein. In some embodiments, the CD8α hinge antibody comprises a VH CDR1, VH CDR2, and VH CDR3 of a CD8α hinge antibody provided herein. In some embodiments, the CD8α hinge antibody comprises a VL CDR1, VL CDR2, and VL CDR3 of a CD8α hinge antibody provided herein. In some embodiments, the CD8α hinge antibody comprises a VH CDR1, VH CDR2, VH CDR3, a VL CDR1, VL CDR2, and VL CDR3 of a CD8α hinge antibody provided herein. In some embodiments, the CD8α hinge antibody comprises a VH of a CD8α hinge antibody provided herein. In some embodiments, the CD8α hinge antibody comprises a VL of a CD8α hinge antibody provided herein. In some embodiments, the CD8α hinge antibody comprises a VH and a VL of a CD8α hinge antibody provided herein.

In another aspect, provided is an antibody that competes for binding to CD8α hinge with a CD8α hinge reference antibody. In another aspect, provided is a CD8α hinge antibody that binds to the same CD8α hinge epitope as a CD8α hinge reference antibody. In another aspect, provided is a CD8α hinge antibody that binds an epitope on CD8α hinge that overlaps with the epitope on CD8α hinge bound by a CD8α hinge reference antibody. In some embodiments, the CD8α hinge reference antibody comprises a VH CDR1, VH CDR2, and VH CDR3 of a CD8α hinge reference antibody provided herein. In some embodiments, the CD8α hinge reference antibody comprises a VL CDR1, VL CDR2, and VL CDR3 of a CD8α hinge reference antibody provided herein. In some embodiments, the CD8α hinge reference antibody comprises a VH CDR1, VH CDR2, VH CDR3, a VL CDR1, VL CDR2, and VL CDR3 of a CD8α hinge reference antibody provided herein. In some embodiments, the CD8α hinge reference antibody comprises a VH of a CD8α hinge reference antibody provided herein. In some embodiments, the CD8α hinge reference antibody comprises a VL of a CD8α hinge reference antibody provided herein. In some embodiments, the CD8α hinge reference antibody comprises a VH and a VL of a CD8α hinge reference antibody provided herein. In some embodiments, the CD8α hinge reference antibody comprises a VH CDR1, VH CDR2, VH CDR3, a VL CDR1, VL CDR2, and VL CDR3 of a CD8α hinge reference antibody provided herein. In some embodiments, the VH CDR1, VH CDR2, VH CDR3, VL CDR1, VL CDR2, and VL CDR3 amino acid sequences of the CD8α hinge reference antibody are according to the Kabat numbering system. In some embodiments, the VH CDR1, VH CDR2, VH CDR3, VL CDR1, VL CDR2, and VL CDR3 amino acid sequences of the CD8α hinge reference antibody are according to the Chothia numbering system. In some embodiments, the VH CDR1, VH CDR2, VH CDR3, VL CDR1, VL CDR2, and VL CDR3 amino acid sequences of the CD8α hinge reference antibody are according to the AbM numbering system. In some embodiments, the VH CDR1, VH CDR2, VH CDR3, VL CDR1, VL CDR2, and VL CDR3 amino acid sequences of the CD8α hinge reference antibody are according to the Contact numbering system. In some embodiments, the VH CDR1, VH CDR2, VH CDR3, VL CDR1, VL CDR2, and VL CDR3 amino acid sequences of the CD8α hinge reference antibody are according to the IMGT numbering system. In certain embodiments, the antibody is a multispecific antibody. In some embodiments, the antibody is a bispecific antibody. In certain embodiments, the CD8α hinge reference antibody is a multispecific antibody. In some embodiments, the CD8α hinge reference antibody is a bispecific antibody.

According to another particular aspect, the invention relates to an isolated anti-CD8α hinge antibody or antigen-binding fragment thereof, wherein the anti-CD8α hinge antibody or antigen-binding fragment thereof is chimeric.

According to another particular aspect, the invention relates to an isolated anti-CD8α hinge antibody or antigen-binding fragment thereof, wherein the anti-CD8α hinge antibody or antigen-binding fragment thereof is human or humanized.

Also provided are isolated nucleic acids encoding the monoclonal antibodies or antigen-binding fragments thereof disclosed herein. Also provided are vectors comprising the isolated nucleic acids encoding the monoclonal antibodies or antigen-binding fragments thereof disclosed herein. Also provided are host cells comprising the vectors comprising the isolated nucleic acids disclosed herein.

In certain aspects, provided is a nucleic acid encoding an antibody that binds to a CD8α hinge provided herein. Also provided is a vector comprising a nucleic acid encoding an antibody that binds to a CD8α hinge provided herein. Also provided is a host cell comprising a vector comprising a nucleic acid encoding an antibody that binds to a CD8α hinge provided herein. Also provided is a kit comprising the vector comprising a nucleic acid encoding an antibody that binds to a CD8α hinge provided herein, and packaging for the same. In another general aspect, the invention relates to an isolated nucleic acid encoding a monoclonal antibody or antigen-binding fragment thereof disclosed herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies provided herein can be altered without changing the amino acid sequences of the proteins.

In another general aspect, the invention relates to a vector comprising an isolated nucleic acid encoding a monoclonal antibody or antigen-binding fragment thereof disclosed herein. In another general aspect, the invention relates to a vector comprising an isolated nucleic acid encoding an antibody or antigen-binding fragment thereof disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments provided herein. Such techniques are well known to those skilled in the art in view of the present disclosure.

In another general aspect, the invention relates to a host cell comprising an isolated nucleic acid encoding a monoclonal antibody or an antigen-binding fragment thereof provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a antibody or antigen-binding fragment thereof disclosed herein. The methods comprise culturing a cell comprising a nucleic acid encoding the antibody or antigen-binding fragment thereof under conditions to produce a antibody or antigen-binding fragment thereof disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

### 5.3 Functional Exogenous Receptor

In one aspect, provided herein is an antibody that is capable of binding to a CD8α hinge region in a functional exogenous receptor. In some embodiments, the functional exogenous receptor comprises an extracellular domain, the CD8α hinge region, a transmembrane domain, and an intracellular signaling domain.

In some embodiments, the functional exogenous receptors can be, for example, chimeric antigen receptor (CAR), engineered T cell receptor (TCR), chimeric TCR (cTCR), and T cell antigen coupler (TAC)-like chimeric receptor. In some embodiments, the functional exogenous receptor is a CAR. In some embodiments, the functional exogenous receptor is a TCR. In some embodiments, the functional exogenous receptor is cTCR. In yet other embodiments, the functional exogenous receptor is a TAC.

### 5.3.1. Extracellular Domain

In some embodiments, the extracellular domain of the present functional exogenous receptor binds to an antigen expressed on the surface of a target cell, such as a tumor cell.

The extracellular domain of the functional exogenous receptors described herein comprises one or more antigen binding domains. The extracellular domain of the functional exogenous receptors provided herein can be in any format as long as the binding of the extracellular domain to its target activates downstream intracellular signals. In some embodiments, the extracellular domain is derived from a naturally occurring receptor (e.g., an ECD of a receptor). In other embodiments, the extracellular domain is not derived from a naturally occurring receptor.

In some embodiments, the extracellular antigen binding domain of the functional exogenous receptor provided herein is monospecific. In other embodiments, the extracellular antigen binding domain of the functional exogenous receptor provided herein is multispecific. In other embodiments, the extracellular antigen binding domain of the functional exogenous receptor provided herein is monovalent. In other embodiments, the extracellular antigen binding domain of the functional exogenous receptor provided herein is multivalent. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multi specific antibodies (e.g., bispecific antibodies so long as they exhibit the desired biological activity), formed from at least two intact antibodies, single chain antibodies, single domain antibodies and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)2 fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present functional exogenous receptors comprise a single-chain Fv (sFv or scFv). ScFvs are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present functional exogenous receptors comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., VHH or VNAR), binding molecules naturally devoid of light chains, single domains (such as VH or VL) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the functional exogenous receptors described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional VH of four chain immunoglobulins. Such a VHH molecule can be derived from antibodies raised in Camelidae species, for example, camel, llama, vicuna, fromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21(11):484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof, e.g., an extracellular antigen binding domain comprising multiple binding domains (e.g., multiple scFvs) in tandem. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multipecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present functional exogenous receptors. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the functional exogenous receptors may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)n, glycine-serine polymers (including, for example, (GS)n, (GSGGS)n, (GGGS)n, and (GGGGS)n, where n is an integer of at least one), glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the functional exogenous receptors provided herein, the disclosure of each of which is incorporated herein by reference.

In some embodiments, the extracellular antigen binding domain provided in the present functional exogenous receptors recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the functional exogenous receptor may be antigens on a single diseased cell or antigens that are expressed on different cells that each contribute to the disease. The antigens targeted by the functional exogenous receptor may be directly or indirectly involved in the diseases.

In some embodiments, the antigen of a target cell is an antigen on the surface of the cancer cell. In some embodiments, the antigen is a tumor-specific antigen, a tumor-associated antigen, or a neoantigen.

In some embodiments, the target cell is a cancer cell, e.g., a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MM), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MM), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is a anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma (MM). In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CIVIL), hairy cell leukemia (HCL), or a myelodysplastic synfrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin synfrome, Sezary synfrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochonfroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Wirthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the functional exogenous receptor provided herein binds to a B cell antigen. In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267, CD268, CD269, CD270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen.

In one embodiment, target of the present functional exogenous receptor is a pathogen. In certain embodiments, the target cell is a cell comprising a pathogen. In some embodiments, the pathogen causes an infectious disease. In some embodiments, the pathogen is a bacteria. In some embodiments, the pathogen is a parasite. In some embodiments, the pathogen is a virus.

### 5.3.2. Transmembrane Domain

The functional exogenous receptor of the present disclosure comprises a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the functional exogenous receptor described herein is derived from a Type I single-pass membrane protein. In some embodiments, transmembrane domains from multi-pass membrane proteins may also be compatible for use in the functional exogenous receptors described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the functional exogenous receptor described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the functional exogenous receptor provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the functional exogenous receptor comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the transmembrane domain of the present functional exogenous receptor is derived from a molecule selected from the group consisting of CD8α, CD4, CD28, CD137, CD80, CD86, CD152 and PD1. In some specific embodiments, the transmembrane domain is from CD8α. In some specific embodiments, the transmembrane domain is from CD28.

### 5.3.3. Intracellular Signaling Domain

The intracellular signaling domain in the functional exogenous receptors provided herein is responsible for activation of at least one of the normal effector functions of the immune effector cell expressing the functional exogenous receptor. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain comprises a primary intracellular signaling domain of an immune effector cell. In some embodiments, the functional exogenous receptor comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and CD66d. In a specific embodiment, the primary intracellular signaling domain is from CD3ζ.

### 5.3.4. Co-Stimulatory Signaling Domain

In some embodiments, the functional exogenous receptor comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the functional exogenous receptor described herein can be an intracellular signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as intracellular signaling domain of CD3z) and one or more co-stimulatory signaling domains. In some embodiments, the one or more co-stimulatory signaling domains and the primary intracellular signaling domain (such as intracellular signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as intracellular signaling domain of CD3z). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the functional exogenous receptors described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the functional exogenous receptors can be the intracellular signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1(LFA-1), CD2, CD7, LIGHT, NKG2C, B7-H3 and ligands that specially bind to CD83. In some specific embodiments, the co-stimulatory signaling domain comprises a cytoplasmic domain of CD28 and/or a cytoplasmic domain of CD137. In some specific embodiments, the co-stimulatory signaling domain comprises a cytoplasmic domain of CD28. In some specific embodiments, the co-stimulatory signaling domain comprises a cytoplasmic domain of CD137.

In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### 5.3.5. Signal Peptide

In certain embodiments, the functional exogenous receptor provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain.

### 5.3.6. CD8α Hinge Region

The antibodies provided herein bind a functional exogenous receptor comprising a hinge region derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

In some embodiments, the CD8α hinge region comprises an amino acid sequence of TTPAPRPPTPAPTIASQPLSLRPEACRPAAGGAVHTRGLDFACD (SEQ ID NO: 209). In some embodiments, the CD8α hinge region comprises an amino acid sequence of STPAPRPPTPAPTIASQPLSLRPEACRPAAGGAVHTRGLDFACD (SEQ ID NO: 210). In some embodiments, the hinge domain is a portion of SEQ ID NO: 209. In some embodiments, the hinge domain is a portion of SEQ ID NO: 210. In some embodiments, the hinge region of the functional exogenous receptor provided herein comprises an amino acid sequence at least 75%, 80%, 85%, 90%, 91%, 92%, 93%, 94%, 95%, 96%, 97%, or 98% identicial to SEQ ID NO: 209. In some embodiments, the hinge region of the functional exogenous receptor provided herein comprises an amino acid sequence at least 75%, 80%, 85%, 90%, 91%, 92%, 93%, 94%, 95%, 96%, 97%, or 98% identicial to SEQ ID NO: 210.

### 5.4. Immune Effector Cells

In another aspect, provided herein is an anti-CD8α antibody capable of binding to an immune effector cell expressing a functional exogenous receptor provided herein.

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells. In some embodiments, the CD8+ T cells lyse antigen-specific target cells upon expressing the CAR and binding to the target cells. In some embodiments, the T cell is a cytotoxic T cell, a helper T cell, a natural killer T cell, a αβ T cell, or a γδT cell.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the polypeptide provided herein into the immune effector cells, such as T cells. In some embodiments, the polypeptide is introduced to the immune effector cells by transfecting any one of the isolated nucleic acids or any one of the vectors described above.

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the polypeptides described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the polypeptide in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, the antibodies of the present disclosure are capable of binding to an immune effector cell expressing a functional exogenous receptor disclosed herein. In some embodiment, the immune effector cell does not express an endogenous CD8α. In other embodiments, the immune effector cell has been engineered to not express an endogenous CD8α.

In another aspect, the antibodies provided herein are capable of binding cells expressing an endogenous CD8α.

### 5.5. Method of Use

In another aspect, provided herein is a method of detecting the presence or measuring the amount of an agent or a molecule comprising a CD8α hinge region. In some embodiments, provided herein is a method of detecting the presence or measuring the amount of CD8α. In some embodiments, provided herein is a method of detecting the presence or measuring the amount of a functional exogenous receptor comprising a CD8α hinge region as described herein. In some embodiments, provided herein is a method of detecting the presence or measuring the amount of a cell (i.e., a naturally occurring immune cell or an engineered cell) that express a molecule comprising a CD8α hinge region.

In one general aspect, provided is a method of targeting or detecting an antigen on the surface of a cell, the method comprising exposing the cell to an anti-CD8α hinge antibody or antigen binding fragment thereof provided herein. Also provided is a method of targeting or detecting an antigen on the surface of a cell, the method comprising exposing the cell to a composition comprising an anti-CD8α hinge antibody or antigen binding fragment thereof provided herein. In some embodiments, the cell expresses an endogenous CD8α. In other embodiments, the cell expresses an engineered receptor comprising a CD8α hinge region or a fragment or a variant thereof.

The functional activity of the antibodies and antigen-binding fragments thereof that bind CD8α hinge can be characterized by methods known in the art and as described herein. Methods for characterizing antibodies and antigen-binding fragments thereof that bind CD8α hinge include, but are not limited to, affinity and specificity assays including Biacore, ELISA, and OctetRed analysis; binding assays to detect the binding of antibodies to target cells by FACS; binding assays to detect the binding of antibodies to CD8α hinge. According to particular embodiments, the methods for characterizing antibodies and antigen-binding fragments thereof that bind CD8α hinge include those described below.

The isolated molecules or the isolated antibodies of the disclosure can be used to selectively enrich, isolate, separate, purify, sort, select, capture or detect CD8+ cells.

The isolated molecules or the isolated antibodies of the disclosure can be used to selectively enrich, isolate, separate, purify, sort, select, capture or detect CD8− cells that express exogenously introduced molecules comprising a CD8α hinge region or a fragment or a variant thereof. In some embodiments, CD8− cells are cells expressing a functional exogenous receptor comprising a CD8α hinge region or a fragment or a variant thereof. In some embodiments, the cells are CAR-NK cells. In other embodiments, the cells are Jurkat CAR-T cells.

The disclosure provides a method of enriching, isolating, separating, purifying, sorting, selecting, capturing or detecting CD8α hinge region expressing cells comprising: providing a sample comprising the CD8α hinge region expressing cells; contacting the sample with an isolated molecule comprising an antigen binding domain, wherein the antigen binding domain specifically binds CD8α hinge; and enriching, isolating, separating, purifying, sorting, selecting, capturing or detecting the CD8α hinge region expressing cell bound to the isolated molecule.

The disclosure provides a method of enriching for CD8α hinge region expressing cells comprising: providing a sample comprising the CD8α hinge region expressing cells; contacting the sample with an isolated molecule comprising an antigen binding domain, wherein the antigen binding domain specifically binds CD8α hinge; and enriching the CD8α hinge region expressing cell bound to the isolated molecule.

The disclosure provides a method of isolating a CD8α hinge region expressing cell comprising: providing a sample comprising the CD8α hinge region expressing cells; contacting the sample with an isolated molecule comprising an antigen binding domain, wherein the antigen binding domain specifically binds CD8α hinge; and isolating the CD8α hinge region expressing cell bound to the isolated molecule.

The disclosure provides a method of separating a CD8α hinge region expressing cell comprising: providing a sample comprising the CD8α hinge region expressing cell; contacting the sample with an isolated molecule comprising an antigen binding domain, wherein the antigen binding domain specifically binds CD8α hinge; and separating the CD8α hinge region expressing cell bound to the isolated molecule.

The disclosure provides a method of purifying a CD8α hinge region expressing cell comprising: providing a sample comprising the CD8α hinge region expressing cell; contacting the sample with an isolated molecule comprising an antigen binding domain, wherein the antigen binding domain specifically binds CD8α hinge; and purifying the CD8α hinge region expressing cell bound to the isolated molecule.

The disclosure provides a method of sorting a CD8α hinge region expressing cell comprising: providing a sample comprising the CD8α hinge region expressing cell;

contacting the sample with an isolated molecule comprising an antigen binding domain, wherein the antigen binding domain specifically binds CD8α hinge; and sorting the CD8α hinge region expressing cell bound to the isolated molecule.

The disclosure provides a method of selecting a CD8α hinge region expressing cell comprising: providing a sample comprising the CD8α hinge region expressing cell; contacting the sample with an isolated molecule comprising an antigen binding domain, wherein the antigen binding domain specifically binds CD8α hinge; and selecting the CD8α hinge region expressing cell bound to the isolated molecule.

The disclosure provides a method of capturing a CD8α hinge region expressing cell comprising: providing a sample comprising the CD8α hinge region expressing cell; contacting the sample with an isolated molecule comprising an antigen binding domain, wherein the antigen binding domain specifically binds CD8α hinge; and capturing the CD8α hinge region expressing cell bound to the isolated molecule.

The disclosure provides a method of detecting a CD8α hinge region expressing cell comprising: providing a sample comprising the CD8α hinge region expressing cell; contacting the sample with an isolated molecule comprising an antigen binding domain, wherein the antigen binding domain specifically binds CD8α hinge; and detecting the CD8α hinge region expressing cell bound to the isolated molecule.

The disclosure also provides a method of enriching, isolating, separating, purifying, sorting, selecting, capturing or detecting a CD8α hinge region expressing cell, comprising: contacting the CD8α hinge region expressing cell with an isolated molecule comprising an antigen binding domain, wherein the antigen binding domain specifically binds CD8α hinge; and enriching, isolating, separating, purifying, sorting, selecting, capturing or detecting the CD8α hinge region expressing cell based on binding of the CD8α hinge region expressing cell to the isolated molecule.

The disclosure also provides a method of enriching a CD8α hinge region expressing cell, comprising: contacting the CD8α hinge region expressing cell with an isolated molecule comprising an antigen binding domain, wherein the antigen binding domain specifically binds CD8α hinge; and enriching the CD8α hinge region expressing cell based on binding of the CD8α hinge region expressing cell to the isolated molecule.

The disclosure also provides a method of isolating a CD8α hinge region expressing cell, comprising: contacting the CD8α hinge region expressing cell with an isolated molecule comprising an antigen binding domain, wherein the antigen binding domain specifically binds CD8α hinge; and isolating the CD8α hinge region expressing cell based on binding of the CD8α hinge region expressing cell to the isolated molecule.

The disclosure also provides a method of separating a CD8α hinge region expressing cell, comprising: contacting the CD8α hinge region expressing cell with an isolated molecule comprising an antigen binding domain, wherein the antigen binding domain specifically binds CD8α hinge; and separating the CD8α hinge region expressing cell based on binding of the CD8α hinge region expressing cell to the isolated molecule.

The disclosure also provides a method of purifying or detecting a CD8α hinge region expressing cell, comprising: contacting the CD8α hinge region expressing cell with an isolated molecule comprising an antigen binding domain, wherein the antigen binding domain specifically binds CD8α hinge; and purifying the CD8α hinge region expressing cell based on binding of the CD8α hinge region expressing cell to the isolated molecule.

The disclosure also provides a method of soting a CD8α hinge region expressing cell, comprising: contacting the CD8α hinge region expressing cell with an isolated molecule comprising an antigen binding domain, wherein the antigen binding domain specifically binds CD8α hinge; and sorting the CD8α hinge region expressing cell based on binding of the CD8α hinge region expressing cell to the isolated molecule.

The disclosure also provides a method of selecting a CD8α hinge region expressing cell, comprising: contacting the CD8α hinge region expressing cell with an isolated molecule comprising an antigen binding domain, wherein the antigen binding domain specifically binds CD8α hinge; and selecting the CD8α hinge region expressing cell based on binding of the CD8α hinge region expressing cell to the isolated molecule.

The disclosure also provides a method of capturing a CD8α hinge region expressing cell, comprising: contacting the CD8α hinge region expressing cell with an isolated molecule comprising an antigen binding domain, wherein the antigen binding domain specifically binds CD8α hinge; and capturing the CD8α hinge region expressing cell based on binding of the CD8α hinge region expressing cell to the isolated molecule.

The disclosure also provides a method of detecting a CD8α hinge region expressing cell, comprising: contacting the CD8α hinge region expressing cell with an isolated molecule comprising an antigen binding domain, wherein the antigen binding domain specifically binds CD8α hinge; and detecting the CD8α hinge region expressing cell based on binding of the CD8α hinge region expressing cell to the isolated molecule.

As used herein the term “CD8α hinge region expressing cells” refers to cells that express a molecule comprising a CD8α hinge region or a fragment or a variant thereof.

In some embodiments, the sample is a blood sample or a tissue sample.

In some embodiments, the method is conducted in suspension or on a solid support.

In some embodiments, the method is conducted using beads, microfluidics, fluorescent cell sorting, chips, columns or surfaces.

In some embodiments, the antigen binding domain comprises a scFv, a Fab, a Fab′, a F(ab′)₂, a Fd, a Fv, a dAb, a VHH, a VH, a VL, a non-antibody scaffold, or fragments thereof.

In some embodiments, the antigen binding domain comprises a fragment of an Fc. In some embodiments, the fragment of the Fc comprises a CH2 domain and a CH3 domain.

In some embodiments, the Fc, the CH2 domain or the CH3 domain is an IgG1, IgG2, IgG3 or IgG4 isotype.

In some embodiments, the antibody or antigen binding domain comprises the VH of SEQ ID NO: 1 and the VL of SEQ ID NO: 2. In some embodiments, the antibody or antigen binding domain comprises the VH of SEQ ID NO: 27 and the VL of SEQ ID NO: 28. In some embodiments, the antibody or antigen binding domain comprises the VH of SEQ ID NO: 53 and the VL of SEQ ID NO: 54. In some embodiments, the antibody or antigen binding domain comprises the VH of SEQ ID NO: 79 and the VL of SEQ ID NO: 80. In some embodiments, the antibody or antigen binding domain comprises the VH of SEQ ID NO: 105 and the VL of SEQ ID NO: 106. In some embodiments, the antibody or antigen binding domain comprises the VH of SEQ ID NO: 131 and the VL of SEQ ID NO: 132. In some embodiments, the antibody or antigen binding domain comprises the VH of SEQ ID NO: 157 and the VL of SEQ ID NO: 158. In some embodiments, the antibody or antigen binding domain comprises the VH of SEQ ID NO: 183 and the VL of SEQ ID NO: 184.

Enrichment, isolation, separation, purification, sorting, selecting, capturing or detecting, or any combination thereof can be done using known technologies such as bead, microfluidics, solid support, columns etc. In general the isolated molecule of the disclosure, when bound to the cell may be separated or visualized using known methods.

### 5.6. Kits

Also provided herein are kits comprising an antibody (e.g., an anti-CD8α antibody) provided herein, or a composition thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to a CD8α hinge region antigen. In specific embodiments, provided herein are panels of antibodies having different association rate constants different dissociation rate constants, different affinities for CD8α hinge region, and/or different specificities for CD8α hinge region. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EMBODIMENTS

This invention provides the following non-limiting embodiments.

Embodiment 1. An antibody that binds a CD8α hinge region, wherein the antibody is capable of binding to a functional exogenous receptor, and wherein the functional exogenous receptor comprises an extracellular domain, the CD8α hinge region, a transmembrane domain, and an intracellular signaling domain.

Embodiment 2. The antibody of embodiment 1, wherein the functional exogenous receptor is a T cell receptor (TCR), a chimeric antigen receptor (CAR), a chimeric TCR (cTCR), or a T cell antigen coupler (TAC)-like chimeric receptor.

Embodiment 3. The antibody of embodiment 1 or 2, wherein the functional exogenous receptor is a CAR.

Embodiment 4. The antibody of embodiment 1 or 2, wherein the functional exogenous receptor is a TCR.

Embodiment 5. The antibody of any one of embodiments 1 to 4, wherein the extracellular domain comprises an antigen binding domain derived from an antibody.

Embodiment 6. The antibody of any one of embodiments 1 to 4, wherein the extracellular domain comprises an antibody fragment.

Embodiment 7. The antibody of any one of embodiments 1 to 4, wherein the extracellular domain comprises a scFv.

Embodiment 8. The antibody of any one of embodiments 1 to 4, wherein the extracellular domain binds an antigen.

Embodiment 9. The antibody of embodiment 8, wherein the antigen is a tumor antigen.

Embodiment 10. The antibody of any one of embodiments 1 to 9, wherein the transmembrane domain is derived from a molecule selected from the group consisting of CD8α, CD4, CD28, CD137, CD80, CD86, CD152 and PD1.

Embodiment 11. The antibody of embodiment 10, wherein the transmembrane domain is from CD8α or CD28.

Embodiment 12. The antibody of any one of embodiments 1 to 11, wherein the intracellular signaling domain comprises a primary intracellular signaling domain of an immune effector cell.

Embodiment 13. The antibody of embodiment 12, wherein the primary intracellular signaling domain is from CD3.

Embodiment 14. The antibody of any one of embodiments 1 to 13, wherein the intracellular signaling domain comprises a co-stimulatory signaling domain.

Embodiment 15. The antibody of embodiment 14, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

Embodiment 16. The antibody of embodiment 15, wherein the co-stimulatory signaling domain comprises a cytoplasmic domain of CD28 and/or a cytoplasmic domain of CD137.

Embodiment 17. The antibody of any one of embodiments 1 to 16, wherein the CD8α hinge region is located between the C-terminus of the extracellular domain and the N-terminus of the transmembrane domain.

Embodiment 18. The antibody of any one of embodiments 1 to 17, wherein the functional exogenous receptor further comprises a signal peptide.

Embodiment 19. The antibody of embodiment 18, wherein the signal peptide is from CD8α.

Embodiment 20. The antibody of any one of embodiments 1 to 19, wherein the antibody is capable of binding to an immune effector cell expressing the functional exogenous receptor, wherein optionally the immune effector cell does not express an endogenous CD8α, or the immune effector cell has been engineered to not express an endogenous CD8α.

Embodiment 21. The antibody of embodiment 20, wherein the immune effector cell is a T cell, a natural killer (NK) cell, a NK T cell, a macrophage, a peripheral blood mononuclear cell (PBMC), a monocyte, a neutrophil, or an eosinophil.

Embodiment 22. The antibody of embodiment 21, wherein the immune effector cell is a T cell and the T cell is a cytotoxic T cell, a helper T cell, a natural killer T cell, a αβ T cell, or a γδT cell; or wherein the immune effect cell is a NK cell.

Embodiment 23. The antibody of any one of embodiments 1 to 22, wherein the CD8α hinge region comprises an amino acid sequence of SEQ ID NO: 209.

Embodiment 24. The antibody of any one of embodiments 1 to 22, wherein the CD8α hinge region comprises an amino acid sequence of SEQ ID NO: 210.

Embodiment 25. The antibody of any one of embodiments 1 to 24, wherein the antibody comprises:

(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:27; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:28;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:54;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:79; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:80;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:106;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:131; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:x132;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:157; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:158; or
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:183; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:184.

Embodiment 26. The antibody of embodiment 25, wherein the VH CDR1, VH CDR2, VH CDR3, VL CDR1, VL CDR2, and VL CDR3 amino acid sequences are according to the Kabat numbering system.

Embodiment 27. The antibody of embodiment 25, wherein the VH CDR1, VH CDR2, VH CDR3, VL CDR1, VL CDR2, and VL CDR3 amino acid sequences are according to the Chothia numbering system.

Embodiment 28. The antibody of embodiment 25, wherein the VH CDR1, VH CDR2, VH CDR3, VL CDR1, VL CDR2, and VL CDR3 amino acid sequences are according to the AbM numbering system.

Embodiment 29. The antibody of embodiment 25, wherein the VH CDR1, VH CDR2, VH CDR3, VL CDR1, VL CDR2, and VL CDR3 amino acid sequences are according to the Contact numbering system.

Embodiment 30. The antibody of embodiment 25, wherein the VH CDR1, VH CDR2, VH CDR3, VL CDR1, VL CDR2, and VL CDR3 amino acid sequences are according to the IMGT numbering system.

Embodiment 31. The antibody of embodiment 25, wherein:

(i) the VH CDR1 comprises the amino acid sequence of SEQ ID NO:3, 9, 15, or 21, the VH CDR2 comprises the amino acid sequence of SEQ ID NO:4, 10, 16, or 22, the VH CDR3 comprises the amino acid sequence of SEQ ID NO:5, 11, 17, or 23, the VL CDR1 comprises the amino acid sequence of SEQ ID NO:6, 12, 18 or 24, the VL CDR2 comprises the amino acid sequence of SEQ ID NO:7, 13, 19, or 25, and the VL CDR3 comprises the amino acid sequence of SEQ ID NO:8, 14, 20, or 26;
(ii) the VH CDR1 comprises the amino acid sequence of SEQ ID NO:29, 35, 41, or 47, the VH CDR2 comprises the amino acid sequence of SEQ ID NO:30, 36, 42, or 48, the VH CDR3 comprises the amino acid sequence of SEQ ID NO:31, 37, 43, or 49, the VL CDR1 comprises the amino acid sequence of SEQ ID NO:32, 38, 44, or 50, the VL CDR2 comprises the amino acid sequence of SEQ ID NO:33, 39, 45, or 51, and the VL CDR3 comprises the amino acid sequence of SEQ ID NO:34, 40, 46, or 52;
(iii) the VH CDR1 comprises the amino acid sequence of SEQ ID NO:55, 61, 67, or 73, the VH CDR2 comprises the amino acid sequence of SEQ ID NO:56, 62, 68, or 74, the VH CDR3 comprises the amino acid sequence of SEQ ID NO:57, 63, 69, or 75, the VL CDR1 comprises the amino acid sequence of SEQ ID NO:58, 64, 70, or 76, the VL CDR2 comprises the amino acid sequence of SEQ ID NO:59, 65, 71, or 77, and the VL CDR3 comprises the amino acid sequence of SEQ ID NO:60, 66, 72, or 78;
(iv) the VH CDR1 comprises the amino acid sequence of SEQ ID NO:81, 87, 93, or 99, the VH CDR2 comprises the amino acid sequence of SEQ ID NO:82, 88, 94, or 100, the VH CDR3 comprises the amino acid sequence of SEQ ID NO:83, 89, 95, or 101, the VL CDR1 comprises the amino acid sequence of SEQ ID NO:84, 90, 96, or 102, the VL CDR2 comprises the amino acid sequence of SEQ ID NO:85, 91, 97, or 103, and the VL CDR3 comprises the amino acid sequence of SEQ ID NO:86, 92, 98, or 104;
(v) the VH CDR1 comprises the amino acid sequence of SEQ ID NO:107, 113, 119, or 125, the VH CDR2 comprises the amino acid sequence of SEQ ID NO:108, 114, 120, or 126, the VH CDR3 comprises the amino acid sequence of SEQ ID NO:109, 115, 121, or 127, the VL CDR1 comprises the amino acid sequence of SEQ ID NO:110, 116, 122, or 128, the VL CDR2 comprises the amino acid sequence of SEQ ID NO:111, 117, 123, or 129, and the VL CDR3 comprises the amino acid sequence of SEQ ID NO:112, 118, 124, or 130;
(vi) the VH CDR1 comprises the amino acid sequence of SEQ ID NO:133, 139, 145, or 151, the VH CDR2 comprises the amino acid sequence of SEQ ID NO:134, 140, 146, or 152, the VH CDR3 comprises the amino acid sequence of SEQ ID NO:135, 141, 147, or 153, the VL CDR1 comprises the amino acid sequence of SEQ ID NO:136, 142, 148, or 154, the VL CDR2 comprises the amino acid sequence of SEQ ID NO:137, 143, 149, or 155, and the VL CDR3 comprises the amino acid sequence of SEQ ID NO:138, 144, 150, or 156;
(vii) the VH CDR1 comprises the amino acid sequence of SEQ ID NO:159, 165, 171, or 177, the VH CDR2 comprises the amino acid sequence of SEQ ID NO:160, 166, 172, or 178, the VH CDR3 comprises the amino acid sequence of SEQ ID NO:161, 167, 173, or 179, the VL CDR1 comprises the amino acid sequence of SEQ ID NO:162, 168, 174, or 180, the VL CDR2 comprises the amino acid sequence of SEQ ID NO:163, 169, 175, or 181, and the VL CDR3 comprises the amino acid sequence of SEQ ID NO:164, 170, 176, or 182;
(viii) the VH CDR1 comprises the amino acid sequence of SEQ ID NO:185, 191, 197, or 203, the VH CDR2 comprises the amino acid sequence of SEQ ID NO:186, 192, 198, or 204, the VH CDR3 comprises the amino acid sequence of SEQ ID NO:187, 193, 199, or 205, the VL CDR1 comprises the amino acid sequence of SEQ ID NO:188, 194, 200, or 206, the VL CDR2 comprises the amino acid sequence of SEQ ID NO:189, 195, 201, or 207, and the VL CDR3 comprises the amino acid sequence of SEQ ID NO:190, 196, 202, or 208.

Embodiment 32. A nucleic acid encoding the antibody of any one of embodiments 1 to 31.

Embodiment 33. A vector comprising the nucleic acid of embodiment 32.

Embodiment 34. A host cell comprising the vector of embodiment 33.

Embodiment 35. An antibody that binds a CD8α hinge region, comprising:

(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:27; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:28;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:54;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:79; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:80;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:106;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:131; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:132;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:157; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:158; or
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:183; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:184.

Embodiment 36. The antibody of embodiment 35, wherein the VH CDR1, VH CDR2, VH CDR3, VL CDR1, VL CDR2, and VL CDR3 amino acid sequences are according to the Kabat numbering system.

Embodiment 37. The antibody of embodiment 35, wherein the VH CDR1, VH CDR2, VH CDR3, VL CDR1, VL CDR2, and VL CDR3 amino acid sequences are according to the Chothia numbering system.

Embodiment 38. The antibody of embodiment 35, wherein the VH CDR1, VH CDR2, VH CDR3, VL CDR1, VL CDR2, and VL CDR3 amino acid sequences are according to the AbM numbering system.

Embodiment 39. The antibody of embodiment 35, wherein the VH CDR1, VH CDR2, VH CDR3, VL CDR1, VL CDR2, and VL CDR3 amino acid sequences are according to the Contact numbering system.

Embodiment 40. The antibody of embodiment 35, wherein the VH CDR1, VH CDR2, VH CDR3, VL CDR1, VL CDR2, and VL CDR3 amino acid sequences are according to the IMGT numbering system.

Embodiment 41. The antibody of embodiment 35, wherein

(i) the VH CDR1 comprises the amino acid sequence of SEQ ID NO:3, 9, 15, or 21, the VH CDR2 comprises the amino acid sequence of SEQ ID NO:4, 10, 16, or 22, the VH CDR3 comprises the amino acid sequence of SEQ ID NO:5, 11, 17, or 23, the VL CDR1 comprises the amino acid sequence of SEQ ID NO:6, 12, 18 or 24, the VL CDR2 comprises the amino acid sequence of SEQ ID NO:7, 13, 19, or 25, and the VL CDR3 comprises the amino acid sequence of SEQ ID NO:8, 14, 20, or 26;
(ii) the VH CDR1 comprises the amino acid sequence of SEQ ID NO:29, 35, 41, or 47, the VH CDR2 comprises the amino acid sequence of SEQ ID NO:30, 36, 42, or 48, the VH CDR3 comprises the amino acid sequence of SEQ ID NO:31, 37, 43, or 49, the VL CDR1 comprises the amino acid sequence of SEQ ID NO:32, 38, 44, or 50, the VL CDR2 comprises the amino acid sequence of SEQ ID NO:33, 39, 45, or 51, and the VL CDR3 comprises the amino acid sequence of SEQ ID NO:34, 40, 46, or 52;
(iii) the VH CDR1 comprises the amino acid sequence of SEQ ID NO:55, 61, 67, or 73, the VH CDR2 comprises the amino acid sequence of SEQ ID NO:56, 62, 68, or 74, the VH CDR3 comprises the amino acid sequence of SEQ ID NO:57, 63, 69, or 75, the VL CDR1 comprises the amino acid sequence of SEQ ID NO:58, 64, 70, or 76, the VL CDR2 comprises the amino acid sequence of SEQ ID NO:59, 65, 71, or 77, and the VL CDR3 comprises the amino acid sequence of SEQ ID NO:60, 66, 72, or 78;
(iv) the VH CDR1 comprises the amino acid sequence of SEQ ID NO:81, 87, 93, or 99, the VH CDR2 comprises the amino acid sequence of SEQ ID NO:82, 88, 94, or 100, the VH CDR3 comprises the amino acid sequence of SEQ ID NO:83, 89, 95, or 101, the VL CDR1 comprises the amino acid sequence of SEQ ID NO:84, 90, 96, or 102, the VL CDR2 comprises the amino acid sequence of SEQ ID NO:85, 91, 97, or 103, and the VL CDR3 comprises the amino acid sequence of SEQ ID NO:86, 92, 98, or 104;
(v) the VH CDR1 comprises the amino acid sequence of SEQ ID NO:107, 113, 119, or 125, the VH CDR2 comprises the amino acid sequence of SEQ ID NO:108, 114, 120, or 126, the VH CDR3 comprises the amino acid sequence of SEQ ID NO:109, 115, 121, or 127, the VL CDR1 comprises the amino acid sequence of SEQ ID NO:110, 116, 122, or 128, the VL CDR2 comprises the amino acid sequence of SEQ ID NO:111, 117, 123, or 129, and the VL CDR3 comprises the amino acid sequence of SEQ ID NO:112, 118, 124, or 130;
(vi) the VH CDR1 comprises the amino acid sequence of SEQ ID NO:133, 139, 145, or 151, the VH CDR2 comprises the amino acid sequence of SEQ ID NO:134, 140, 146, or 152, the VH CDR3 comprises the amino acid sequence of SEQ ID NO:135, 141, 147, or 153, the VL CDR1 comprises the amino acid sequence of SEQ ID NO:136, 142, 148, or 154, the VL CDR2 comprises the amino acid sequence of SEQ ID NO:137, 143, 149, or 155, and the VL CDR3 comprises the amino acid sequence of SEQ ID NO:138, 144, 150, or 156;
(vii) the VH CDR1 comprises the amino acid sequence of SEQ ID NO:159, 165, 171, or 177, the VH CDR2 comprises the amino acid sequence of SEQ ID NO:160, 166, 172, or 178, the VH CDR3 comprises the amino acid sequence of SEQ ID NO:161, 167, 173, or 179, the VL CDR1 comprises the amino acid sequence of SEQ ID NO:162, 168, 174, or 180, the VL CDR2 comprises the amino acid sequence of SEQ ID NO:163, 169, 175, or 181, and the VL CDR3 comprises the amino acid sequence of SEQ ID NO:164, 170, 176, or 182;
(viii) the VH CDR1 comprises the amino acid sequence of SEQ ID NO:185, 191, 197, or 203, the VH CDR2 comprises the amino acid sequence of SEQ ID NO:186, 192, 198, or 204, the VH CDR3 comprises the amino acid sequence of SEQ ID NO:187, 193, 199, or 205, the VL CDR1 comprises the amino acid sequence of SEQ ID NO:188, 194, 200, or 206, the VL CDR2 comprises the amino acid sequence of SEQ ID NO:189, 195, 201, or 207, and the VL CDR3 comprises the amino acid sequence of SEQ ID NO:190, 196, 202, or 208.

Embodiment 42. The antibody of any one of embodiments 35 to 41, comprising:

(i) a VH comprising the amino acid sequence of SEQ ID NO: 1, and a VL comprising the amino acid sequence of SEQ ID NO: 2;
(ii) a VH comprising the amino acid sequence of SEQ ID NO: 27, and a VL comprising the amino acid sequence of SEQ ID NO: 28;
(iii) a VH comprising the amino acid sequence of SEQ ID NO: 53, and a VL comprising the amino acid sequence of SEQ ID NO: 54;
(iv) a VH comprising the amino acid sequence of SEQ ID NO:79, and a VL comprising the amino acid sequence of SEQ ID NO:80;
(v) a VH comprising the amino acid sequence of SEQ ID NO: 105, and a VL comprising the amino acid sequence of SEQ ID NO: 106;
(vi) a VH comprising the amino acid sequence of SEQ ID NO: 131, and a VL comprising the amino acid sequence of SEQ ID NO: 132;
(vii) a VH comprising the amino acid sequence of SEQ ID NO: 157, and a VL comprising the amino acid sequence of SEQ ID NO:158; or
(viii) a VH comprising the amino acid sequence of SEQ ID NO:183, and a VL comprising the amino acid sequence of SEQ ID NO:184.

Embodiment 43. The antibody of any one of embodiments 35 to 42, wherein the antibody is a humanized antibody.

Embodiment 44. The antibody of any one of embodiments 35 to 42, wherein the antibody is a human antibody.

Embodiment 45. The antibody of any one of embodiments 35 to 44, wherein the antibody is an IgG antibody.

Embodiment 46. The antibody of embodiment 45, wherein the IgG antibody is an IgG1, IgG2, IgG3, or IgG4 antibody.

Embodiment 47. The antibody of any one of embodiments 35 to 46, wherein the antibody comprises a kappa light chain.

Embodiment 48. The antibody of any one of embodiments 35 to 46, wherein the antibody comprises a lambda light chain.

Embodiment 49. The antibody of any one of embodiments 35 to 48, wherein the antibody is a monoclonal antibody.

Embodiment 50. The antibody of any one of embodiments 35 to 39, wherein the antibody is a multivalent antibody.

Embodiment 51. The antibody of any one of embodiments 35 to 49, wherein the antibody is a multispecific antibody.

Embodiment 52. The antibody of any one of embodiments 35 to 51, wherein the antibody is genetically fused to or chemically conjugated to an agent.

Embodiment 53. A nucleic acid encoding the antibody of any one of embodiments 35 to 52.

Embodiment 54. A vector comprising the nucleic acid of embodiment 53.

Embodiment 55. A host cell comprising the vector of embodiment 54.

Embodiment 56. A kit comprising the antibody of any one of embodiments 1 to 31 and 35 to 52.

Embodiment 57. A method for detecting and/or enriching an agent comprising a CD8α hinge region in a system comprising contacting the system with an antibody that binds a CD8α hinge region.

Embodiment 58. A method for detecting and/or enriching a functional exogenous receptor in a system comprising contacting the system with an antibody that binds a CD8α hinge region, wherein the functional exogenous receptor comprises an extracellular domain, the CD8α hinge region, a transmembrane domain, and an intracellular signaling domain, wherein optionally the functional exogenous receptor is expressed in an immune effector cell.

Embodiment 59. A method for detecting and/or enriching immune effector cells expressing a functional exogenous receptor comprising contacting a population of cells with an antibody that binds a CD8α hinge region, wherein the functional exogenous receptor comprises an extracellular domain, the CD8α hinge region, a transmembrane domain, and an intracellular signaling domain.

Embodiment 60. The method of embodiment 58 or embodiment 59, the functional exogenous receptor is a T cell receptor (TCR), a chimeric antigen receptor (CAR), a chimeric TCR (cTCR), or a T cell antigen coupler (TAC)-like chimeric receptor.

Embodiment 61. The method of any one of embodiments 57 to 60, wherein the antibody that binds the CD8α hinge region is the antibody of any one of embodiments 1 to 31 and 35 to 52.

Embodiment 62. A system comprising a means for binding a CD8α hinge region in a functional exogenous receptor, wherein the functional exogenous receptor comprises an extracellular domain, the CD8α hinge region, a transmembrane domain, and an intracellular signaling domain.

Embodiment 63. A system comprising a means for binding a CD8α hinge region in a functional exogenous receptor expressed in an immune effector cell, wherein the functional exogenous receptor comprises an extracellular domain, the CD8α hinge region, a transmembrane domain, and an intracellular signaling domain.

Embodiment 64. A system comprising a means for binding an immune effector cell expressing a functional exogenous receptor, wherein the functional exogenous receptor comprises an extracellular domain, a CD8α hinge region, a transmembrane domain, and an intracellular signaling domain.

Embodiment 65. The system of any one of embodiments 62 to 64, wherein the functional exogenous receptor is a T cell receptor (TCR), a chimeric antigen receptor (CAR), a chimeric TCR (cTCR), or a T cell antigen coupler (TAC)-like chimeric receptor.

Embodiment 66. The system of embodiment 65, wherein the functional exogenous receptor is a CAR.

Embodiment 67. The system of embodiment 65, wherein the functional exogenous receptor is a TCR.

Embodiment 68. The system of any one of embodiments 62 to 67, wherein the immune effector cell does not express an endogenous CD8α, or the immune effector cell has been engineered to not express an endogenous CD8α.

Embodiment 69. The system of embodiment 68, wherein the immune effector cell is a T cell, a natural killer (NK) cell, a NK T cell, a macrophage, a peripheral blood mononuclear cell (PBMC), a monocyte, a neutrophil, or an eosinophil.

Embodiment 70. The system of embodiment 69, wherein the immune effector cell is a T cell and the T cell is a cytotoxic T cell, a helper T cell, a natural killer T cell, a αβ T cell, or a γδT cell.

Embodiment 71. The system of embodiment 69, wherein the immune effect cell is a NK cell.

Embodiment 72. A method for detecting and/or enriching a functional exogenous receptor using the system of any one of embodiments 62 to 71.

Embodiment 73. A method for detecting and/or enriching immune effector cells expressing a functional exogenous receptor using the system of any one of embodiments 62 to 71.

Representative amino acid sequences of antibodies provided herein are shown in the Tables provided in the Examples section and are contemplated as certain embodiments. In addition, representative nucleic acid sequences encoding antibodies provided herein are shown in the Tables provided in the Examples section and are contemplated as certain embodiments.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### Example 1: Generation of CD8α Hinge Antibodies

Immunogen. Recombinant human CD8alpha/beta heterodimer protein (cat #9358-CD) was obtained from R&D Systems, Inc. The amino acid sequence of the heterodimeric protein is listed in Table 2.

TABLE 2
Amino acid sequence of recombinant human CD8α/β heterodimer protein

| Name | Protein ID | Sequence | SEQ ID NO |
|---|---|---|---|
| Recombinant | rhCD8α | SQFRVSPLDRTWNLGETVELKCQVLLSNPTSG | 213 |
| human | (Ser22-Asp182) | CSWLFQPRGAAASPTFLLYLSQNKPKAAEGLD | |
| CD8α/β | Accession | TQRFSGKRLGDTFVLTLSDFRRENEGYYFCSA | |
| heterodimer | #P01732 | LSNSIMYFSHFVPVFLPAKPTTTPAPRPPTPA | |
| protein (cat #: | | PTIASQPLSLRPEACRPAAGGAVHTRGLDFAC | |
| 9358-CD) | | D-[proprietary R&D Systems, Inc. | |
| | | acidic tails]-HHHHHH | |
| | rhCD8β | NSVLQQTPAYIKVQTNKMVMLSCEAKISLSNM | 214 |
| | (Asn19-Pro170) | RIYWLRQRQAPSSDSHHEFLALWDSAKGTIHG | |
| | Accession | EEVEQEKIAVFRDASRFILNLTSVKPEDSGIY | |
| | #P10966 | FCMIVGSPELTFGKGTQLSVVDFLPTTAQPTK | |
| | | KSTLKKRVCRLPRPETQKGPLCSP- | |
| | | [proprietary R&D Systems, Inc. | |
| | | basic tails]-DYKDDDDK | |

Immunization and hybridoma screening. Wild-type (WT) mice with 6 different MEW combinations was immunized with recombinant human CD8alpha/beta heterodimer protein using rapid immunization protocol. Mouse sera were collected on day 22. Serum titer were determined by LUMINEX using immunogen and flow cytometry using human primary CD8+ T cells. Eight mice were selected for cell fusion based on serum titer. Approximately 1000 hybridoma supernatants were shown binding to immunogen and human primary CD8+ cells. To identify CD8α hinge specific binders, a Jurkat cell line expressing CAR-T with CD8α hinge as the spacer in CAR design was generated to screen the hybridoma supernatants along with Jurkat cell line (CD8−) and Sup-T1 cell line (CD8+). A total of 30 hybridomas were identified as positive binders to CD8α hinge expressed on the Jurkat CAR-T cells. After limit dilution, hybridoma clones were screened by flow cytometry using the Jurkat CAR-T cells and Jurkat cells as well as Sup-T1 cells.

mAb expression. A total of 30 positive hybridoma clones were selected for V-gene recovery and a total of 8 diverse sequences were identified. The nucleic acid sequences of encoding variable regions were subcloned into mammalian expression vectors containing constant region of human IgG1 or human kappa light chain constant region (Table 3). The mAbs were expressed by transient transfection in Chinese hamster ovary cell line. The antibodies were purified by MAB SELECT SURE Protein A column (GE healthcare, Piscataway, N.J.) (Brown, Bottomley et al. 1998). Eluted samples were neutralized and buffer-exchanged to PBS. Samples were analyzed by polyacrylamide gel electrophoresis under reducing and non-reducing conditions and the intact mass was confirmed by mass spectrometry.

TABLE 3
VH/VL and Heavy and Light Chain sequences for CD8α hinge antibody
variants

| | VH AA | VL AA | | |
|---|---|---|---|---|
| Protein ID | sequence | sequence | HC AA sequence | LC AA sequence |
| CD8B575 | QVQLQQSGG | DIVMTQSQK | QVQLQQSGGGLVQPGGSLKLSCAAS | DIVMTQSQKFMSTTIED |
| | GLVQPGGSL | FMSTTIEDR | GFTFSNYYMSWARQTPEKRLEWVAY | RVSITCKASQNVGTAVA |
| | KLSCAASGF | VSITCKASQ | ISNDGDATYYPDSVKGRFTISRDNA | WYQQKPGQSPKLLIYST |
| | TFSNYYMSW | NVGTAVAWY | KNTLYLQMSSLKSEDTAMYYCTRED | SNRYTGVPDRFTGSGSG |
| | ARQTPEKRL | QQKPGQSPK | YYSDYWFASWGQGTTVTVSSASTKG | TDFTLTISNIQSEDLAD |
| | EWVAYISND | LLIYSTSNR | PSVFPLAPSSKSTSGGTAALGCLVK | YFCQQYSSYPLTFGGGT |
| | GDATYYPDS | YTGVPDRFT | DYFPEPVTVSWNSGALTSGVHTFPA | KLEIKRTVAAPSVFIFP |
| | VKGRFTISR | GSGSGTDFT | VLQSSGLYSLSSVVTVPSSSLGTQT | PSDEQLKSGTASVVCLL |
| | DNAKNTLYL | LTISNIQSE | YICNVNHKPSNTKVDKKVEPKSCDK | NNFYPREAKVQWKVDNA |
| | QMSSLKSED | DLADYFCQQ | THTCPPCPAPELLGGPSVFLFPPKP | LQSGNSQESVTEQDSKD |
| | TAMYYCTRE | YSSYPLTFG | KDTLMISRTPEVTCVVVDVSHEDPE | STYSLSSTLTLSKADYE |
| | DYYSDYWFA | GGTKLEIK | VKFNWYVDGVEVHNAKTKPREEQYN | KHKVYACEVTHQGLSSP |
| | SWGQGTTVT | (SEQ ID | STYRVVSVLTVLHQDWLNGKEYKCK | VTKSFNRGEC (SEQ ID |
| | VSS (SEQ ID | NO: 184) | VSNKALPAPIEKTISKAKGQPREPQ | NO: 230) |
| | NO: 183) | | VYTLPPSREEMTKNQVSLTCLVKGF | |
| | | | YPSDIAVEWESNGQPENNYKTTPPV | |
| | | | LDSDGSFFLYSKLTVDKSRWQQGNV | |
| | | | FSCSVMHEALHNHYTQKSLSLSPGK | |
| | | | (SEQ ID NO: 229) | |
| CD8B607 | EVQLQQSGA | DIQMTQTPL | EVQLQQSGAELVRPGASVNLSCTAS | DIQMTQTPLTLSVTIGQ |
| | ELVRPGASV | TLSVTIGQP | GFNFKDDYMHWVKQRPEQGLEWIGW | PASISCKSSQSLLDSDG |
| | NLSCTASGF | ASISCKSSQ | VDPENGDAAYASKFQGKATITADTS | ETYLNWLLQRPGQSPKR |
| | NFKDDYMHW | SLLDSDGET | SNTAYLHLSSLTSEDAAVYYCITIF | LIYLVSKLDSGVPDRFT |
| | VKQRPEQGL | YLNWLLQRP | RSYWGQGTTLTVSSASTKGPSVFPL | GSGSGTDFTLKISRVEA |
| | EWIGWVDPE | GQSPKRLIY | APSSKSTSGGTAALGCLVKDYFPEP | EDLGVYYCWQGTHFPLT |
| | NGDAAYASK | LVSKLDSGV | VTVSWNSGALTSGVHTFPAVLQSSG | FGGGTKLEIKRTVAAPS |
| | FQGKATITA | PDRFTGSGS | LYSLSSVVTVPSSSLGTQTYICNVN | VFIFPPSDEQLKSGTAS |
| | DTSSNTAYL | GTDFTLKIS | HKPSNTKVDKKVEPKSCDKTHTCPP | VVCLLNNFYPREAKVOW |
| | HLSSLTSED | RVEAEDLGV | CPAPELLGGPSVFLFPPKPKDTLMI | KVDNALQSGNSQESVTE |
| | AAVYYCITI | YYCWQGTHF | SRTPEVTCVVVDVSHEDPEVKFNWY | QDSKDSTYSLSSTLTLS |
| | FRSYWGQGT | PLTFGGGTK | VDGVEVHNAKTKPREEQYNSTYRVV | KADYEKHKVYACEVTHQ |
| | TLTVSS | LEIK (SEQ | SVLTVLHQDWLNGKEYKCKVSNKAL | GLSSPVTKSFNRGEC |
| | (SEQ ID | ID NO: 28) | PAPIEKTISKAKGQPREPQVYTLPP | (SEQ ID NO: 218) |
| | NO: 27) | | SREEMTKNQVSLTCLVKGFYPSDIA | |
| | | | VEWESNGQPENNYKTTPPVLDSDGS | |
| | | | FFLYSKLTVDKSRWQQGNVFSCSVM | |
| | | | HEALHNHYTQKSLSLSPGK (SEQ ID | |
| | | | NO: 217) | |
| CD8B611 | EVQLQQSGA | DIQMTQSPL | EVQLQQSGAELVRPGASVNLSCTAS | DIQMTQSPLTLSVTIGQ |
| | ELVRPGASV | TLSVTIGQP | GFNFKDDYMHWVKQRPEQGLEWIGW | PASISCKSSQSLLDSDG |
| | NLSCTASGF | ASISCKSSQ | IDPENGDAAYASKFQGKATITADTS | ETYLNWLLQRPGQSPKR |
| | NFKDDYMHW | SLLDSDGET | SNTAYLQLSSLTSEDTAVYYCITIF | LIYLVSKLDSGVPDRFT |
| | VKQRPEQGL | YLNWLLQRP | RSYWGQGTTLTVSSASTKGPSVFPL | GSGSGTDFTLKISRVEA |
| | EWIGWIDPE | GQSPKRLIY | APSSKSTSGGTAALGCLVKDYFPEP | EDLGVYYCWQGTHFPLT |
| | NGDAAYASK | LVSKLDSGV | VTVSWNSGALTSGVHTFPAVLQSSG | FGGGTKLEIKRTVAAPS |
| | FQGKATITA | PDRFTGSGS | LYSLSSVVTVPSSSLGTQTYICNVN | VFIFPPSDEQLKSGTAS |
| | DTSSNTAYL | GTDFTLKIS | HKPSNTKVDKKVEPKSCDKTHTCPP | VVCLLNNFYPREAKVQW |
| | QLSSLTSED | RVEAEDLGV | CPAPELLGGPSVFLFPPKPKDTLMI | KVDNALQSGNSQESVTE |
| | TAVYYCITI | YYCWQGTHF | SRTPEVTCVVVDVSHEDPEVKFNWY | QDSKDSTYSLSSTLTLS |
| | FRSYWGQGT | PLTFGGGTK | VDGVEVHNAKTKPREEQYNSTYRVV | KADYEKHKVYACEVTHQ |
| | TLTVSS | LEIK (SEQ | SVLTVLHQDWLNGKEYKCKVSNKAL | GLSSPVTKSFNRGEC |
| | (SEQ ID | ID NO: | PAPIEKTISKAKGQPREPQVYTLPP | (SEQ ID NO: 224) |
| | NO: 105) | 106) | SREEMTKNQVSLTCLVKGFYPSDIA | |
| | | | VEWESNGQPENNYKTTPPVLDSDGS | |
| | | | FFLYSKLTVDKSRWQQGNVFSCSVM | |
| | | | HEALHNHYTQKSLSLSPGK (SEQ ID | |
| | | | NO: 223) | |
| CD8B612 | DVQLQESGA | DIVMTQTPL | DVQLQESGAELVKPGASVILSCKAS | DIVMTQTPLSLPVSLGD |
| | ELVKPGASV | SLPVSLGDQ | GYTFTDYTIHWLKQTSGQGLEWIGW | QASISCRSSQNIVHSNG |
| | ILSCKASGY | ASISCRSSQ | FYPGSGSVKYNEKYNEKFRDKATLT | NTYLEWYLQKPGQSPKL |
| | TFTDYTIHW | NIVHSNGNT | ADKSSRTVYMELIRLTSEDSAVYFC | LIYRVSKRFSGVPDRFS |
| | LKQTSGQGL | YLEWYLQKP | ARHGSYYDDAMDYWGQGTTVTVSSA | GSGSGTDFTLKITRVEA |
| | EWIGWFYPG | GQSPKLLIY | STKGPSVFPLAPSSKSTSGGTAALG | EDLGVYYCFQGSHVPWT |
| | SGSVKYNEK | RVSKRFSGV | CLVKDYFPEPVTVSWNSGALTSGVH | FGGGTKLEIKRTVAAPS |
| | YNEKFRDKA | PDRFSGSGS | TFPAVLQSSGLYSLSSVVTVPSSSL | VFIFPPSDEQLKSGTAS |
| | TLTADKSSR | GTDFTLKIT | GTQTYICNVNHKPSNTKVDKKVEPK | VVCLLNNFYPREAKVQW |
| | TVYMELIRL | RVEAEDLGV | SCDKTHTCPPCPAPELLGGPSVFLF | KVDNALQSGNSQESVTE |
| | TSEDSAVYF | YYCFQGSHV | PPKPKDTLMISRTPEVTCVVVDVSH | QDSKDSTYSLSSTLTLS |
| | CARHGSYYD | PWTFGGGTK | EDPEVKFNWYVDGVEVHNAKTKPRE | KADYEKHKVYACEVTHQ |
| | DAMDYWGQG | LEIK (SEQ | EQYNSTYRVVSVLTVLHQDWLNGKE | GLSSPVTKSFNRGEC |
| | TTVTVSS | ID NO: 80) | YKCKVSNKALPAPIEKTISKAKGQP | (SEQ ID NO: 222) |
| | (SEQ ID | | REPQVYTLPPSREEMTKNQVSLTCL | |
| | NO: 79) | | VKGFYPSDIAVEWESNGQPENNYKT | |
| | | | TPPVLDSDGSFFLYSKLTVDKSRWQ | |
| | | | QGNVFSCSVMHEALHNHYTQKSLSL | |
| | | | SPGK (SEQ ID NO: 221) | |
| CD8B613 | DVQLQESGA | DVQMIQSPL | DVQLQESGAELVKPGASVILSCKAS | DVQMIQSPLSLPVSLGD |
| | ELVKPGASV | SLPVSLGDQ | GYTFTDYTIHWLKQTSGQGLEWIGW | QASISCRSSQNIVHSNG |
| | ILSCKASGY | ASISCRSSQ | FYPGSGSVKYNEKYNEKFRDKATLT | NTYLEWYLQKPGQSPKL |
| | TFTDYTIHW | NIVHSNGNT | ADKSSRTVYMELIRLTSEDSAVYFC | LIYRVSKRFSGVPDRFS |
| | LKQTSGQGL | YLEWYLQKP | ARHGSYYDDAMDYWGQGTTVTVSSA | GSGSGTDFTLKITRVEA |
| | EWIGWFYPG | GQSPKLLIY | STKGPSVFPLAPSSKSTSGGTAALG | EDLGVYYCFQGSHVPWT |
| | SGSVKYNEK | RVSKRFSGV | CLVKDYFPEPVTVSWNSGALTSGVH | FGGGTKLEIKRTVAAPS |
| | YNEKFRDKA | PDRFSGSGS | TFPAVLQSSGLYSLSSVVTVPSSSL | VFIFPPSDEQLKSGTAS |
| | TLTADKSSR | GTDFTLKIT | GTQTYICNVNHKPSNTKVDKKVEPK | VVCLLNNFYPREAKVQW |
| | TVYMELIRL | RVEAEDLGV | SCDKTHTCPPCPAPELLGGPSVFLF | KVDNALQSGNSQESVTE |
| | TSEDSAVYF | YYCFQGSHV | PPKPKDTLMISRTPEVTCVVVDVSH | QDSKDSTYSLSSTLTLS |
| | CARHGSYYD | PWTFGGGTK | EDPEVKFNWYVDGVEVHNAKTKPRE | KADYEKHKVYACEVTHQ |
| | DAMDYWGQG | LEIK (SEQ | EQYNSTYRVVSVLTVLHQDWLNGKE | GLSSPVTKSFNRGEC |
| | TTVTVSS | ID NO: 54) | YKCKVSNKALPAPIEKTISKAKGQP | (SEQ ID NO: 220) |
| | (SEQ ID | | REPQVYTLPPSREEMTKNQVSLTCL | |
| | NO: 53) | | VKGFYPSDIAVEWESNGQPENNYKT | |
| | | | TPPVLDSDGSFFLYSKLTVDKSRWQ | |
| | | | QGNVFSCSVMHEALHNHYTQKSLSL | |
| | | | SPGK (SEQ ID NO: 219) | |
| CD8B617 | EVQLQQSGA | DIQMTQSPL | EVQLQQSGAELVRPGASVNLSCTAS | DIQMTQSPLTLSVTIGQ |
| | ELVRPGASV | TLSVTIGQP | GFNFKDDYMHWVKQRPEQGLEWIGW | PASISCKSSQSLLDSDG |
| | NLSCTASGF | ASISCKSSQ | VDPENGDAAYASKFQGKATITADTS | ETYLNWLLQRPGQSPKR |
| | NFKDDYMHW | SLLDSDGET | SNTAYLHLSSLTSEDAAVYYCITIF | LIYLVSKLDSGVPDRFT |
| | VKQRPEQGL | YLNWLLQRP | RSYWGQGTTLTVSSASTKGPSVFPL | GSGSGTDFTLKISRVEA |
| | EWIGWVDPE | GQSPKRLIY | APSSKSTSGGTAALGCLVKDYFPEP | EDLGVYYCWQGTHFPLT |
| | NGDAAYASK | LVSKLDSGV | VTVSWNSGALTSGVHTFPAVLQSSG | FGGGTKLEIKRTVAAPS |
| | FQGKATITA | PDRFTGSGS | LYSLSSVVTVPSSSLGTQTYICNVN | VFIFPPSDEQLKSGTAS |
| | DTSSNTAYL | GTDFTLKIS | HKPSNTKVDKKVEPKSCDKTHTCPP | VVCLLNNFYPREAKVQW |
| | HLSSLTSED | RVEAEDLGV | CPAPELLGGPSVFLFPPKPKDTLMI | KVDNALQSGNSQESVTE |
| | AAVYYCITI | YYCWQGTHF | SRTPEVTCVVVDVSHEDPEVKFNWY | QDSKDSTYSLSSTLTLS |
| | FRSYWGQGT | PLTFGGGTK | VDGVEVHNAKTKPREEQYNSTYRVV | KADYEKHKVYACEVTHQ |
| | TLTVSS | LEIK (SEQ | SVLTVLHQDWLNGKEYKCKVSNKAL | GLSSPVTKSFNRGEC |
| | (SEQ ID | ID NO: 2) | PAPIEKTISKAKGQPREPQVYTLPP | (SEQ ID NO: 216) |
| | NO: 1) | | SREEMTKNQVSLTCLVKGFYPSDIA | |
| | | | VEWESNGQPENNYKTTPPVLDSDGS | |
| | | | FFLYSKLTVDKSRWQQGNVFSCSVM | |
| | | | HEALHNHYTQKSLSLSPGK (SEQ ID | |
| | | | NO: 215) | |
| CD8B622 | QIQLVQSGA | DIVMTQTPL | QIQLVQSGAELVKPGASVILSCKAS | DIVMTQTPLSLPVSLGD |
| | ELVKPGASV | SLPVSLGDQ | GYTFTDYTIHWLKQTSGQGLEWIGW | QASISCRSSQNIVHSNG |
| | ILSCKASGY | ASISCRSSQ | FYPGSGSVKYNEKYNEKFRDKATLT | NTYLEWYLQKPGQSPKL |
| | TFTDYTIHW | NIVHSNGNT | ADKSSRTVYMELIRLTSEDSAVYFC | LIYRVSKRFSGVPDRFS |
| | LKQTSGQGL | YLEWYLQKP | ARHGSYYDDAMDYWGQGTTLTVSSA | GSGSGTDFTLKITRVEA |
| | EWIGWFYPG | GQSPKLLIY | STKGPSVFPLAPSSKSTSGGTAALG | EDLGVYYCFQGSHVPWT |
| | SGSVKYNEK | RVSKRFSGV | CLVKDYFPEPVTVSWNSGALTSGVH | FGGGTKLEIKRTVAAPS |
| | YNEKFRDKA | PDRFSGSGS | TFPAVLQSSGLYSLSSVVTVPSSSL | VFIFPPSDEQLKSGTAS |
| | TLTADKSSR | GTDFTLKIT | GTQTYICNVNHKPSNTKVDKKVEPK | VVCLLNNFYPREAKVQW |
| | TVYMELIRL | RVEAEDLGV | SCDKTHTCPPCPAPELLGGPSVFLF | KVDNALQSGNSQESVTE |
| | TSEDSAVYF | YYCFQGSHV | PPKPKDTLMISRTPEVTCVVVDVSH | QDSKDSTYSLSSTLTLS |
| | CARHGSYYD | PWTFGGGTK | EDPEVKFNWYVDGVEVHNAKTKPRE | KADYEKHKVYACEVTHQ |
| | DAMDYWGQG | LEIK (SEQ | EQYNSTYRVVSVLTVLHQDWLNGKE | GLSSPVTKSFNRGEC |
| | TTLTVSS | ID NO: | YKCKVSNKALPAPIEKTISKAKGQP | (SEQ ID NO: 226) |
| | (SEQ ID | 132) | REPQVYTLPPSREEMTKNQVSLTCL | |
| | NO: 131) | | VKGFYPSDIAVEWESNGQPENNYKT | |
| | | | TPPVLDSDGSFFLYSKLTVDKSRWQ | |
| | | | QGNVFSCSVMHEALHNHYTQKSLSL | |
| | | | SPGK (SEQ ID NO: 225) | |
| CD8B623 | QIQLVQSGA | DVVMTQTPL | QIQLVQSGAELVKPGASVILSCKAS | DVVMTQTPLSLPVSLGD |
| | ELVKPGASV | SLPVSLGDQ | GYTFTDYTIHWLKQTSGQGLEWIGW | QASISCRSSQNIVHSNG |
| | ILSCKASGY | ASISCRSSQ | FYPGSGSVKYNEKYNEKFRDKATLT | NTYLEWYLQKPGQSPKL |
| | TFTDYTIHW | NIVHSNGNT | ADKSSRTVYMELIRLTSEDSAVYFC | LIYRVSKRFSGVPDRFS |
| | LKQTSGQGL | YLEWYLQKP | ARHGSYYDDAMDYWGQGTTLTVSSA | GSGSGTDFTLKITRVEA |
| | EWIGWFYPG | GQSPKLLIY | STKGPSVFPLAPSSKSTSGGTAALG | EDLGVYYCFQGSHVPWT |
| | SGSVKYNEK | RVSKRFSGV | CLVKDYFPEPVTVSWNSGALTSGVH | FGGGTKLEIKRTVAAPS |
| | YNEKFRDKA | PDRFSGSGS | TFPAVLQSSGLYSLSSVVTVPSSSL | VFIFPPSDEQLKSGTAS |
| | TLTADKSSR | GTDFTLKIT | GTQTYICNVNHKPSNTKVDKKVEPK | VVCLLNNFYPREAKVOW |
| | TVYMELIRL | RVEAEDLGV | SCDKTHTCPPCPAPELLGGPSVFLF | KVDNALQSGNSQESVTE |
| | TSEDSAVYF | YYCFQGSHV | PPKPKDTLMISRTPEVTCVVVDVSH | QDSKDSTYSLSSTLTLS |
| | CARHGSYYD | PWTFGGGTK | EDPEVKFNWYVDGVEVHNAKTKPRE | KADYEKHKVYACEVTHQ |
| | DAMDYWGQG | LEIK (SEQ | EQYNSTYRVVSVLTVLHQDWLNGKE | GLSSPVTKSFNRGEC |
| | TTLTVSS | ID NO: | YKCKVSNKALPAPIEKTISKAKGQP | (SEQ ID NO: 228) |
| | (SEQ ID | 158) | REPQVYTLPPSREEMTKNQVSLTCL | |
| | NO: 157) | | VKGFYPSDIAVEWESNGQPENNYKT | |
| | | | TPPVLDSDGSFFLYSKLTVDKSRWQ | |
| | | | QGNVFSCSVMHEALHNHYTQKSLSL | |
| | | | SPGK (SEQ ID NO: 227) | |

Table 4 shows the Kabat HCDR1, HCDR2 and HCDR3 of the anti-CD8α Hinge antibodies. Table 5 shows the Kabat LCDR1, LCDR2 and LCDR3 of the anti-CD8α hinge antibodies.

TABLE 4
HCDR1, HCDR2 and HCDR3 amino acid sequences of anti-CD8α hinge
antibodies using Kabat delineation.

| | Kabat HCDR1 | | Kabat HCDR2 | | Kabat HCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| CD8B617 | DDYMH | 3 | WVDPENGDAAYASKFQG | 4 | IFRSY | 5 |
| CD8B607 | DDYMH | 29 | WVDPENGDAAYASKFQG | 30 | IFRSY | 31 |
| CD8B613 | DYTIH | 55 | WFYPGSGSVKYNEKYNEKFRD | 56 | HGSYYDDAMDY | 57 |
| CD8B612 | DYTIH | 81 | WFYPGSGSVKYNEKYNEKFRD | 82 | HGSYYDDAMDY | 83 |
| CD8B611 | DDYMH | 107 | WIDPENGDAAYASKFQG | 108 | IFRSY | 109 |
| CD8B622 | DYTIH | 133 | WFYPGSGSVKYNEKYNEKFRD | 134 | HGSYYDDAMDY | 135 |
| CD8B623 | DYTIH | 159 | WFYPGSGSVKYNEKYNEKFRD | 160 | HGSYYDDAMDY | 161 |
| CD8B575 | NYYMS | 185 | YISNDGDATYYPDSVKG | 186 | EDYYSDYWFAS | 187 |

TABLE 5
LCDR1, LCDR2 and LCDR3 amino acid sequences of anti-CD8a hinge
antibodies using Kabat delineation.

| | Kabat LCDR1 | Kabat LCDR2 | | | Kabat LCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| CD8B617 | KSSQSLLDSDGETYLN | 6 | LVSKLDS | 7 | WQGTHFPLT | 8 |
| CD8B607 | KSSQSLLDSDGETYLN | 32 | LVSKLDS | 33 | WQGTHFPLT | 34 |
| CD8B613 | RSSQNIVHSNGNTYLE | 58 | RVSKRFS | 59 | FQGSHVPWT | 60 |
| CD8B612 | RSSQNIVHSNGNTYLE | 84 | RVSKRFS | 85 | FQGSHVPWT | 86 |
| CD8B611 | KSSQSLLDSDGETYLN | 110 | LVSKLDS | 111 | WQGTHFPLT | 112 |
| CD8B622 | RSSQNIVHSNGNTYLE | 136 | RVSKRFS | 137 | FQGSHVPWT | 138 |
| CD8B623 | RSSQNIVHSNGNTYLE | 162 | RVSKRFS | 163 | FQGSHVPWT | 164 |
| CD8B575 | KASQNVGTAVA | 188 | STSNRYT | 189 | QQYSSYPLT | 190 |

Table 6 shows the Chothia HCDR1, HCDR2 and HCDR3 of the anti-CD8α Hinge antibodies. Table 7 shows the Chothia LCDR1, LCDR2 and LCDR3 of the anti-CD8α hinge antibodies.

TABLE 6
HCDR1, HCDR2 and HCDR3 amino acid sequences of anti-CD8α hinge
antibodies using Chothia delineation.

| | Chothia HCDR1 | | Chothia HCDR2 | | Chothia HCDR3 | |
|---|---|---|---|---|---|---|
| | | SEQ | | SEQ | | SEQ |
| | | ID | | ID | | ID |
| mAb name | Sequence | NO: | Sequence | NO: | Sequence | NO: |
| CD8B617 | GFNFKDD | 9 | DPENGD | 10 | IFRSY | 11 |
| CD8B607 | GFNFKDD | 35 | DPENGD | 36 | IFRSY | 37 |
| CD8B613 | GYTFTDY | 61 | YPGSGSVKYN | 62 | HGSYYDDAMDY | 63 |
| CD8B612 | GYTFTDY | 87 | YPGSGSVKYN | 88 | HGSYYDDAMDY | 89 |
| CD8B611 | GFNFKDD | 113 | DPENGD | 114 | IFRSY | 115 |
| CD8B622 | GYTFTDY | 139 | YPGSGSVKYN | 140 | HGSYYDDAMDY | 141 |
| CD8B623 | GYTFTDY | 165 | YPGSGSVKYN | 166 | HGSYYDDAMDY | 167 |
| CD8B575 | GFTFSNY | 191 | SNDGDA | 192 | EDYYSDYWFAS | 193 |

TABLE 7
LCDR1, LCDR2 and LCDR3 amino acid sequences of anti-CD8α hinge
antibodies using Chothia delineation.

| | Chothia LCDR1 | | Chothia LCDR2 | | Chothia LCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| CD8B617 | KSSQSLLDSDGETYLN | 12 | LVSKLDS | 13 | WQGTHFPLT | 14 |
| CD8B607 | KSSQSLLDSDGETYLN | 38 | LVSKLDS | 39 | WQGTHFPLT | 40 |
| CD8B613 | RSSQNIVHSNGNTYLE | 64 | RVSKRFS | 65 | FQGSHVPWT | 66 |
| CD8B612 | RSSQNIVHSNGNTYLE | 90 | RVSKRFS | 91 | FQGSHVPWT | 92 |
| CD8B611 | KSSQSLLDSDGETYLN | 116 | LVSKLDS | 117 | WQGTHFPLT | 118 |
| CD8B622 | RSSQNIVHSNGNTYLE | 142 | RVSKRFS | 143 | FQGSHVPWT | 144 |
| CD8B623 | RSSQNIVHSNGNTYLE | 168 | RVSKRFS | 169 | FQGSHVPWT | 170 |
| CD8B575 | KASQNVGTAVA | 194 | STSNRYT | 195 | QQYSSYPLT | 196 |

Table 8 shows the IMGT HCDR1, HCDR2 and HCDR3 of the anti-CD8α Hinge antibodies. Table 9 shows the IMGT LCDR1, LCDR2 and LCDR3 of the anti-CD8α hinge antibodies.

TABLE 8
HCDR1, HCDR2 and HCDR3 amino acid sequences of anti-CD8α hinge
antibodies using IMGT delineation.

| | IMGT HCDR1 | | IMGT HCDR2 | | IMGT HCDR3 | |
|---|---|---|---|---|---|---|
| | | SEQ | | SEQ | | SEQ |
| | | ID | | ID | | ID |
| mAb name | Sequence | NO: | Sequence | NO: | Sequence | NO: |
| CD8B617 | GFNFKDDY | 15 | VDPENGDA | 16 | ITIFRSY | 17 |
| CD8B607 | GFNFKDDY | 41 | VDPENGDA | 42 | ITIFRSY | 43 |
| CD8B613 | GYTFTDYT | 67 | FYPGSGSVKYNE | 68 | ARHGSYYDDAMDY | 69 |
| CD8B612 | GYTFTDYT | 93 | FYPGSGSVKYNE | 94 | ARHGSYYDDAMDY | 95 |
| CD8B611 | GFNFKDDY | 119 | IDPENGDA | 120 | ITIFRSY | 121 |
| CD8B622 | GYTFTDYT | 145 | FYPGSGSVKYNE | 146 | ARHGSYYDDAMDY | 147 |
| CD8B623 | GYTFTDYT | 171 | FYPGSGSVKYNE | 172 | ARHGSYYDDAMDY | 173 |
| CD8B575 | GFTFSNYY | 197 | ISNDGDAT | 198 | TREDYYSDYWFAS | 199 |

TABLE 9
LCDR1, LCDR2 and LCDR3 amino acid sequences of anti-CD8α hinge
antibodies using IMGT delineation.

| | IMGT LCDR1 | | IMGT LCDR2 | | IMGT LCDR3 | |
|---|---|---|---|---|---|---|
| | | SEQ | | SEQ | | SEQ |
| | | ID | | ID | | ID |
| mAb name | Sequence | NO: | Sequence | NO: | Sequence | NO: |
| CD8B617 | QSLLDSDGETY | 18 | LVS | 19 | WQGTHFPLT | 20 |
| CD8B607 | QSLLDSDGETY | 44 | LVS | 45 | WQGTHFPLT | 46 |
| CD8B613 | QNIVHSNGNTY | 70 | RVS | 71 | FQGSHVPWT | 72 |
| CD8B612 | QNIVHSNGNTY | 96 | RVS | 97 | FQGSHVPWT | 98 |
| CD8B611 | QSLLDSDGETY | 122 | LVS | 123 | WQGTHFPLT | 124 |
| CD8B622 | QNIVHSNGNTY | 148 | RVS | 149 | FQGSHVPWT | 150 |
| CD8B623 | QNIVHSNGNTY | 174 | RVS | 175 | FQGSHVPWT | 176 |
| CD8B575 | QNVGTA | 200 | STS | 201 | QQYSSYPLT | 202 |

Table 10 shows the ABM HCDR1, HCDR2 and HCDR3 of the anti-CD8α Hinge antibodies. Table 11 shows the ABM LCDR1, LCDR2 and LCDR3 of the anti-CD8α hinge antibodies.

TABLE 10
HCDR1, HCDR2 and HCDR3 amino acid sequences of anti-CD8α hinge
antibodies using ABM delineation.

| | ABM HCDR1 | | ABM HCDR2 | | ABM HCDR3 | |
|---|---|---|---|---|---|---|
| | | SEQ | | SEQ | | SEQ |
| | | ID | | ID | | ID |
| mAb name | Sequence | NO: | Sequence | NO: | Sequence | NO: |
| CD8B617 | GFNFKDDYMH | 21 | WVDPENGDAA | 22 | IFRSY | 23 |
| CD8B607 | GFNFKDDYMH | 47 | WVDPENGDAA | 48 | IFRSY | 49 |
| CD8B613 | GYTFTDYTIH | 73 | WFYPGSGSVKYNEK | 74 | HGSYYDDAMDY | 75 |
| CD8B612 | GYTFTDYTIH | 99 | WFYPGSGSVKYNEK | 100 | HGSYYDDAMDY | 101 |
| CD8B611 | GFNFKDDYMH | 125 | WIDPENGDAA | 126 | IFRSY | 127 |
| CD8B622 | GYTFTDYTIH | 151 | WFYPGSGSVKYNEK | 152 | HGSYYDDAMDY | 153 |
| CD8B623 | GYTFTDYTIH | 177 | WFYPGSGSVKYNEK | 178 | HGSYYDDAMDY | 179 |
| CD8B575 | GFTFSNYYMS | 203 | YISNDGDATY | 204 | EDYYSDYWFAS | 205 |

TABLE 11
LCDR1, LCDR2 and LCDR3 amino acid sequences of anti-CD8α hinge
antibodies using ABM delineation.

| | ABM LCDR1 | | ABM LCDR2 | | ABM LCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| CD8B617 | KSSQSLLDSDGETYLN | 24 | LVSKLDS | 25 | WQGTHFPLT | 26 |
| CD8B607 | KSSQSLLDSDGETYLN | 50 | LVSKLDS | 51 | WQGTHFPLT | 52 |
| CD8B613 | RSSQNIVHSNGNTYLE | 76 | RVSKRFS | 77 | FQGSHVPWT | 78 |
| CD8B612 | RSSQNIVHSNGNTYLE | 102 | RVSKRFS | 103 | FQGSHVPWT | 104 |
| CD8B611 | KSSQSLLDSDGETYLN | 128 | LVSKLDS | 129 | WQGTHFPLT | 130 |
| CD8B622 | RSSQNIVHSNGNTYLE | 154 | RVSKRFS | 155 | FQGSHVPWT | 156 |
| CD8B623 | RSSQNIVHSNGNTYLE | 180 | RVSKRFS | 181 | FQGSHVPWT | 182 |
| CD8B575 | KASQNVGTAVA | 206 | STSNRYT | 207 | QQYSSYPLT | 208 |

### Example 2: Dose-Dependent Binding of Anti-CD8α Hinge mAbs to Jurkat CAR-T Cells

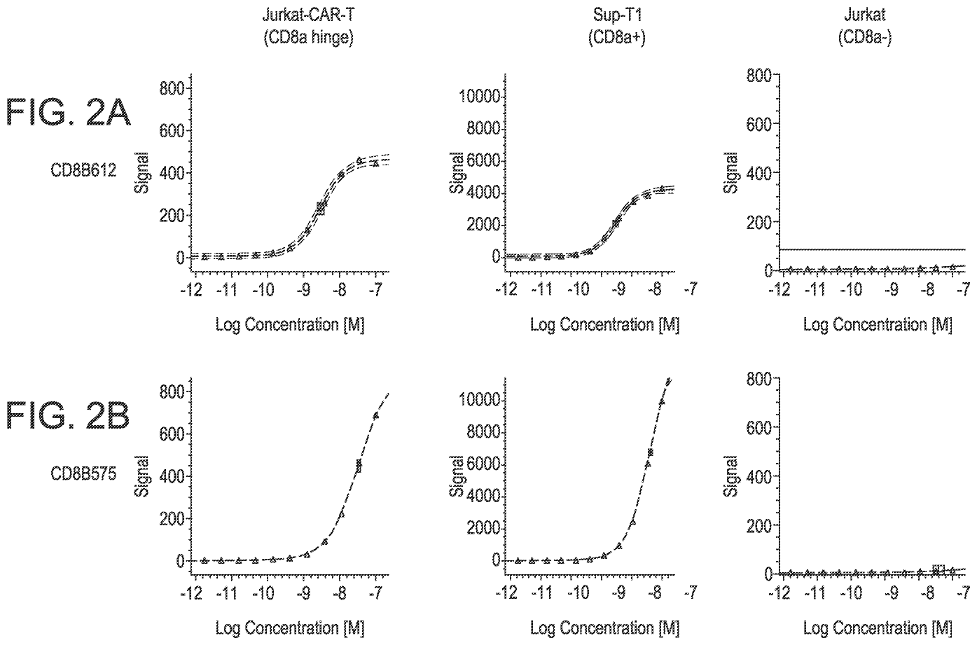

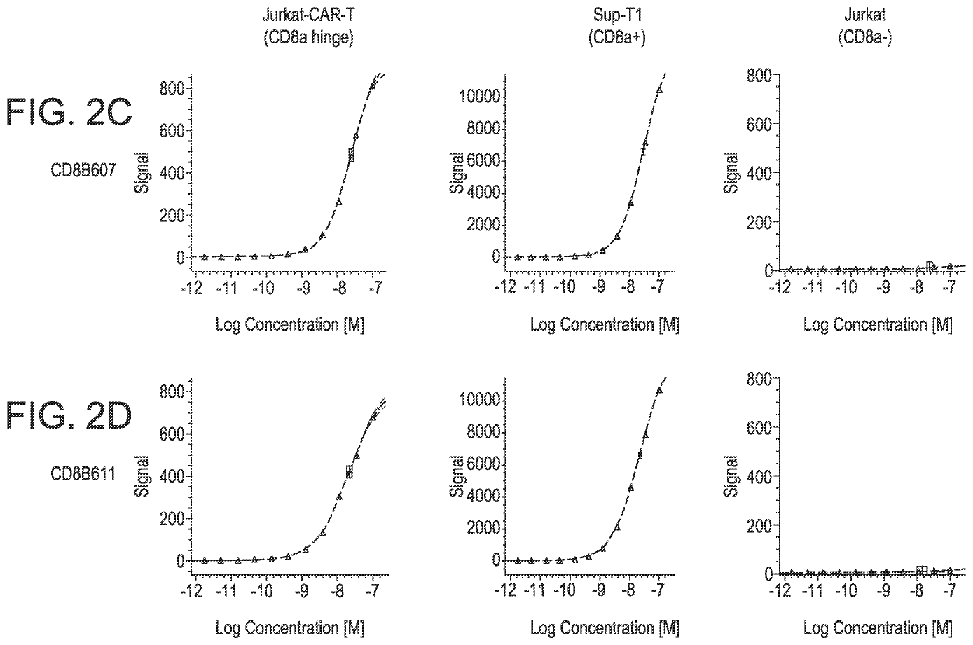

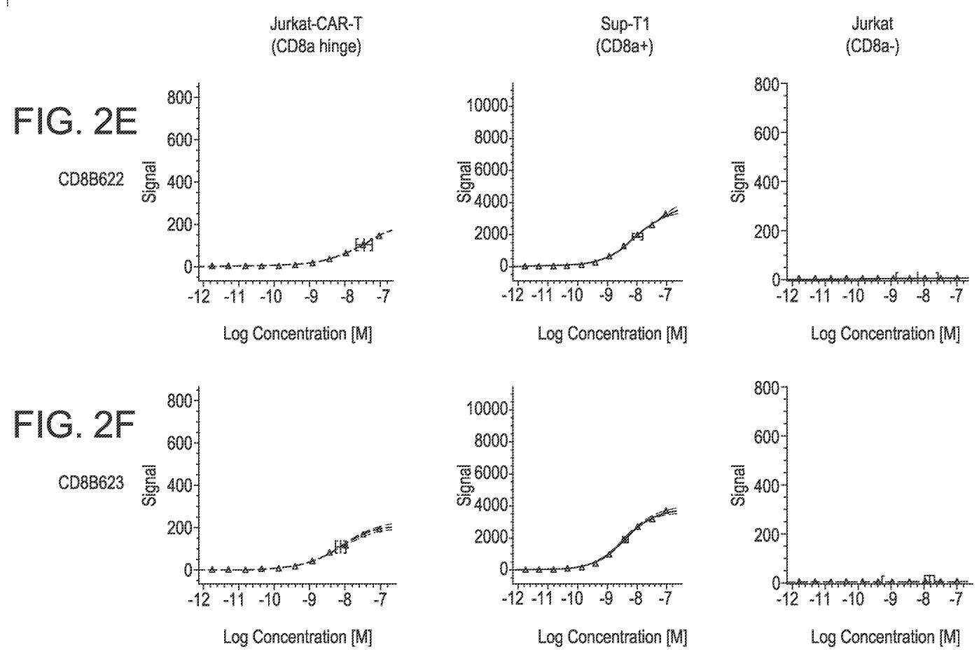

mAbs were 1:3 serially diluted ranging from 100 nM to 1.7 pM and were incubated with cell mixture containing Jurkat CAR-T cells (unstained), Jurkat cells (CTV dye stained) and Sup-T1 cells (CSFE dye stained) in assay media (RPMI 1640+10% HI FBS+Pen/strep) for 1 hr at 37° C. Cells were washed twice prior to incubation with A647 conjugated anti human IgG Fc specific secondary detection antibody (Jackson Immunoresearch, Cat #109-606-098) for 20 min at 25° C. Cells were then washed and analyzed by iQue screener (Intellicyt®). ForeCyt software was used for data analysis. The ratio of geomean of AF647 fluorescent signal from sample over the isotype control antibody (Signal) was used for the dose-dependent plot (FIGS. 2A-2F). EC50 of the mAbs are shown in Table 12.

TABLE 12
EC50 of CD8α hinge binders to Jurkat CAR-T cells

| Compound ID | EC50 (nM) |
|---|---|
| CD8B613 | 1.9 |
| CD8B612 | 2.9 |
| CD8B623 | 7.8 |
| CD8B611 | 21.6 |
| CD8B617 | 23.7 |
| CD8B607 | 24.7 |
| CD8B575 | 32.6 |
| CD8B622.001 | 37.0 |

### Example 3: Binding of CD8α Hinge Binders to Primary CD8+ T Cells

Frozen PBMCs were thawed and resuspended in FACS buffer (PBS+2% FBS). Cells were then incubated with Fc block for 15 min at 4 C prior to incubation with 2 μg/mL of each CD8α hinge binders at room temperature for 45 min. Cells were washed and then incubated with an anti-Human IgG, Fcγ fragment specific antibody (Jackson Immunoresearch, Cat #109-606-098) and a cocktail of anti-human CD3, CD4, CD8, CD45RA and CD27 at 4° C. for 30 min to define T cell subpopulations. After staining, cells were washed with FACS buffer twice and resuspended in 100 μl PBS containing DAPI for acquisition. Cells were first gated on FSC/SSC, followed by live cell gating (DAPI negative cells) followed by singlet gating. CD3 T cells were identified using FSC versus CD3 dot plot. Within the CD3 T cells, CD4 and CD8 T cells were gated. Naïve, Effector, EM and CM cells were identified within the CD8 T cells using CD45RA and CD27 dot plot. Within each of the cell types, the secondary only control was used to set the gates to identify the positive stained population. The percentage of positive cells in CD8+ T cell population with binding to CD8α hinge binders were quantified and summarized in Table 13.

TABLE 13
Binding of anti-CD8α hinge binders to CD8+ primary T cells

| | Binding in | | Binding+ in | | Binding+ in | |
|---|---|---|---|---|---|---|
| Sample- | CD8+ cells (%) | | EM (%) | | EMRA (%) | |
| (ug/ml) | Donor 1 | Donor 2 | Donor 1 | Donor 2 | Donor 1 | Donor 2 |
| CD8B575 | 84.41 | 96.39 | 69.55 | 77.88 | 81.35 | 92.81 |
| CD8B607 | 87.45 | 97.66 | 82.81 | 87.4 | 87.92 | 97.14 |
| CD8B611 | 88.83 | 98.22 | 88.5 | 91.53 | 91.05 | 97.79 |
| CD8B612 | 71.62 | 32.85 | 65.06 | 72.83 | 67.57 | 7.67 |
| CD8B613 | 73.82 | 28.14 | 70.48 | 75.9 | 71.97 | 8.76 |
| CD8B617 | 88.53 | 97.76 | 86.24 | 89.13 | 90.3 | 97.28 |
| CD8B622 | 63.08 | 16.68 | 50.44 | 50.31 | 60.61 | 4.25 |
| CD8B623 | 65.31 | 19.54 | 56.54 | 60.63 | 63.34 | 5.38 |

### Example 4: Application of the Cd8α Hinge Binders for Jurkat CAR-T Detection

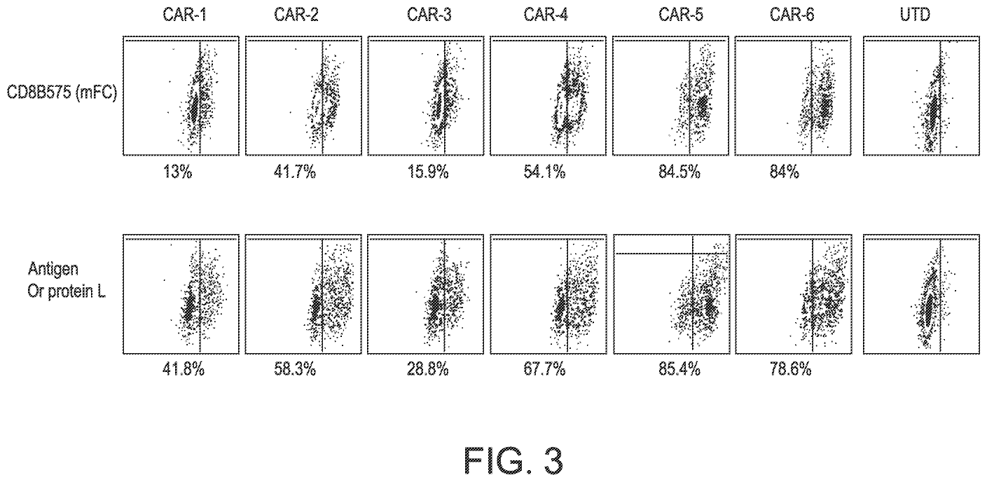

Jurkat report cell line (CD8−) has been used as a valuable tool for screening optimal CAR-T constructs (Smith et al., Sci. Transl, Med. 2019, 11 (485)). To test if CD8α hinge binders can be used to measure CAR expression on Jurkat cells, a panel of lentiviral plasmids that encode various CAR-T molecules with CD8α hinge as the spacer were transduced in Jurkat cells, CAR expression was determined by flow cytometry either with the mouse FC version of CD8B575 (purified from mouse hybridoma clone) followed by fluorescently labelled second antibody. For comparison, CAR expression on Jurkat cell lines was also measured by fluorescently conjugated protein L or antigen. The percentage of CD79b CAR+Jurkat cells (% positive) was analyzed and shown in FIG. 3 using the untransduced cells as negative controls. CD8B575 equivalent showed comparable CAR-T detection sensitivity as antigen and protein L.

### Example 5: Application of the Cd8α Hinge Binders for Nk Car Detection

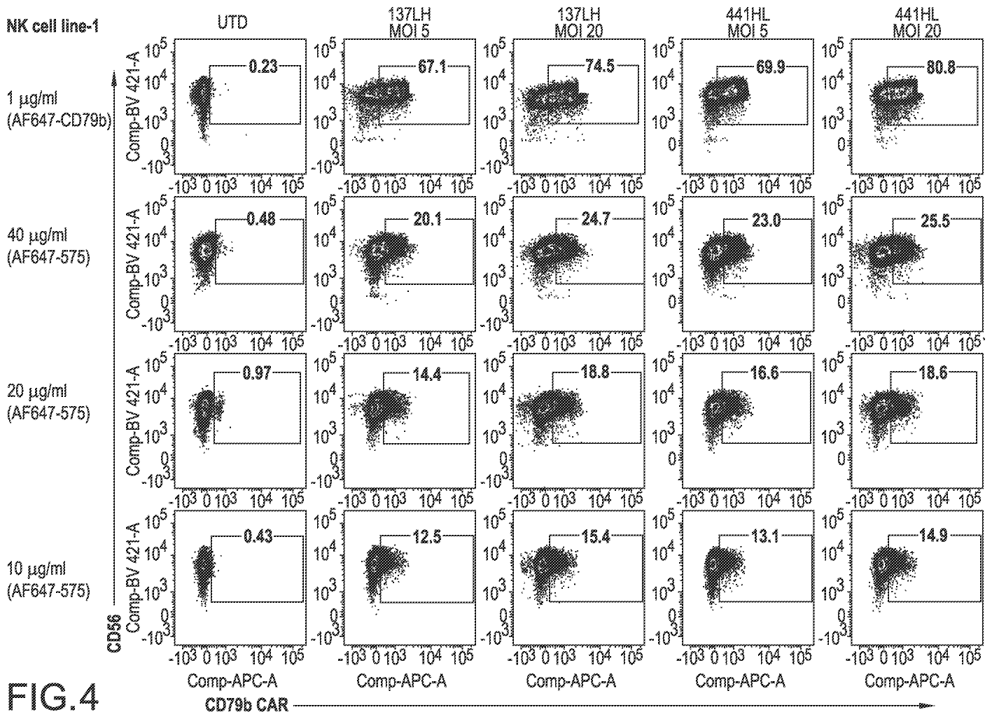

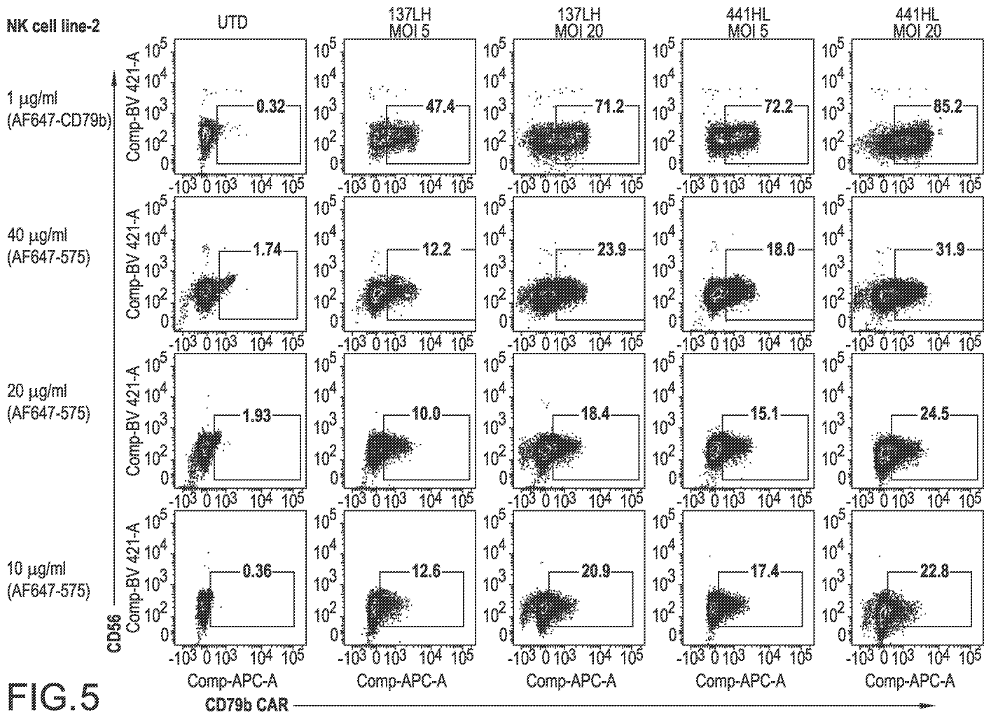

Two NK cell lines, NK-92 and NKL, both of which are CD3−CD8−, were transduced with two lentiviral plasmids encoding CD79b CAR (MOI 5 and 20). 5 days post transduction, CAR expression was determined by flow cytometric analysis using either the AF674 conjugated antigen, AF647-CD79b (1 ug/ml) or the AF647 conjugated anti-CD8α hinge binder CD8B575. Untransduced (UTD) NK cell lines which do not express CAR were used as negative controls for data analysis. The percentage of CD79b CAR+NK cells (% positive) was analyzed and shown in FIG. 4 and FIG. 5 respectively for each cell line. As shown, AF647−CD8B575 bound to NK CAR cells in a concentration-dependent manner. AF674 conjugated antigen showed higher level of CAR expression. This result may be attributed to the relative low affinity of CD8B575 to CD8α hinge region is 45 aa in length and the accessibility of the CD8α hinge on the NK cell membrane.

### Example 6: Binding of CD8α Hinge Binders to Soluble Car Extracellular Domain

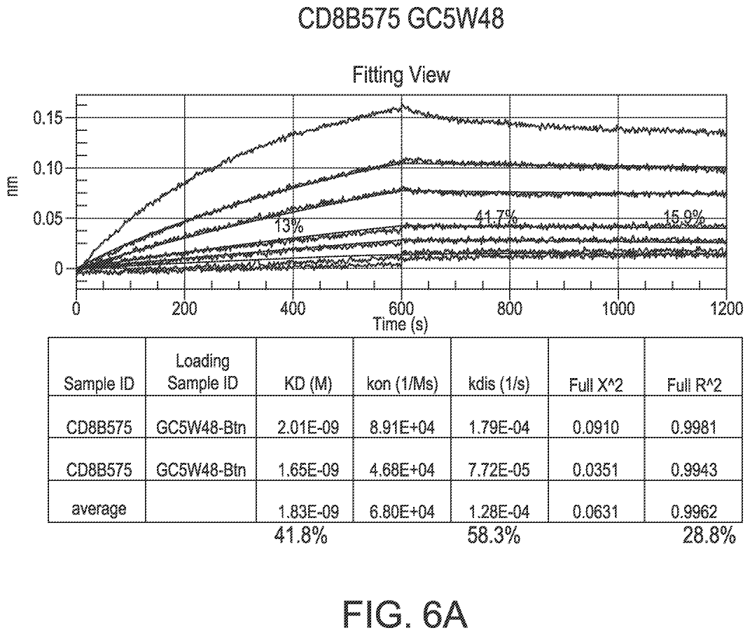

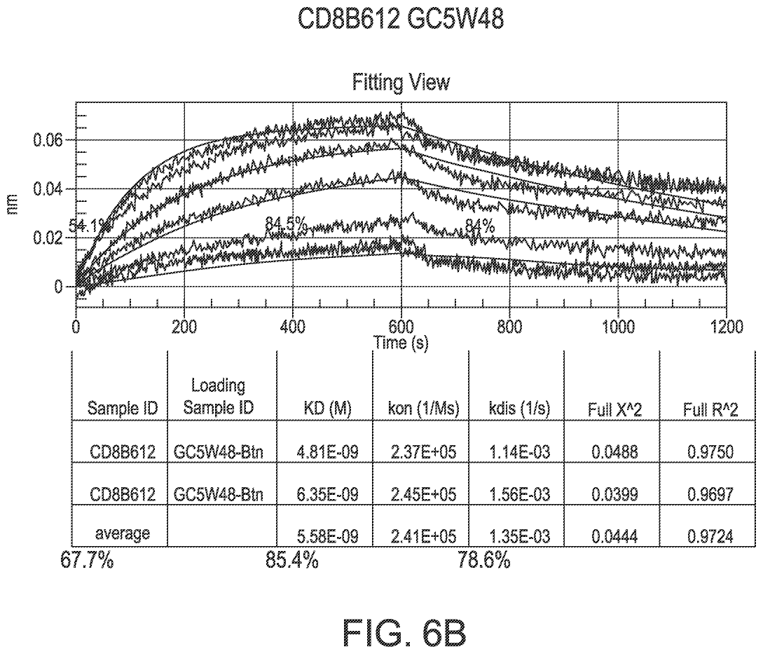

To determine the binding affinity and kinetics of mAbs to CD8α hinge, soluble CAR extracellular domains (ECD) containing scFv and CD8α hinge was generated for Bio-layer interferometry (BLI) analysis using OctetRed384 (Sartorius). The CAR ECD was biotinylated and loaded to streptavidin sensor tip. Then mAbs were associated to the sensor and followed by disassociation in the assay buffer. Data was analyzed by Octet software (Sartorius). CD8B575 and CD8B612 bound to soluble CAR ECD with 1.83 nM and 5.58 nM affinity respectively while other mAbs did not bind (FIG. 6 and Table 14).

TABLE 14
Binding kinetics of CD8α hinge binders
to soluble CAR extracellular domain

| Protein ID | ka (1/Ms) | kd (1/s) | KD (M) |
|---|---|---|---|
| CD8B575 | 1.83E−09 | 6.80E+04 | 1.28E−04 |
| CD8B612 | 5.58E−09 | 2.41E+05 | 1.35E−03 |

### Example 7: Select CD8α Hinge Binders for Ada Assay Development

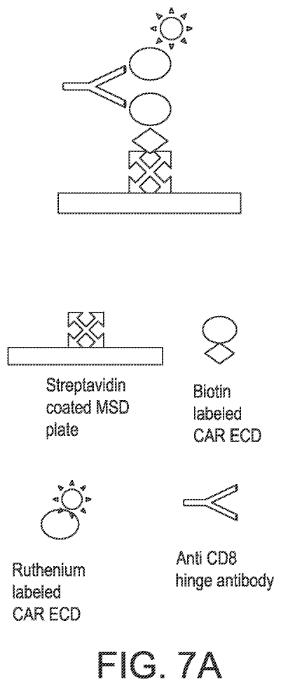

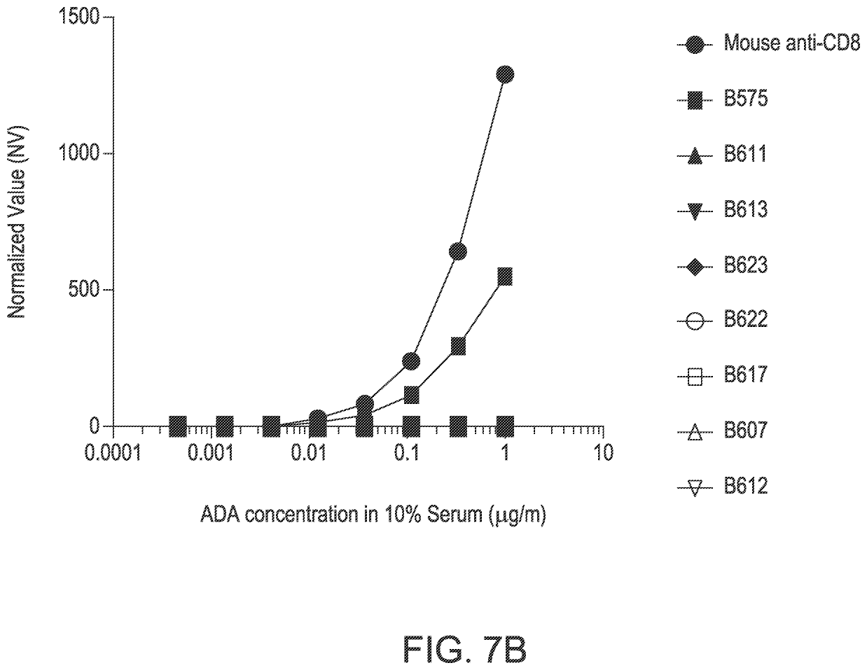

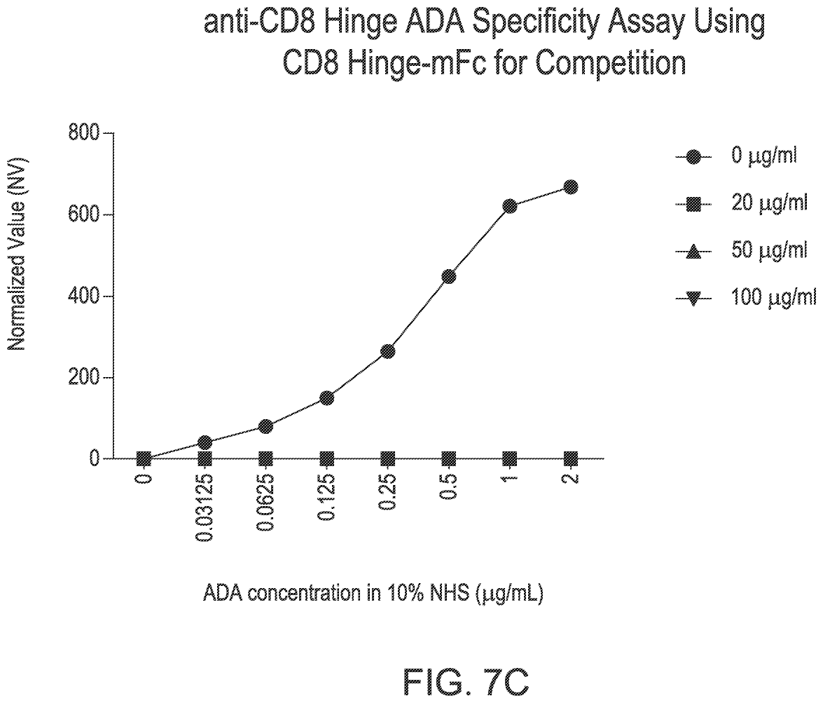

As shown in FIG. 7A, the anti-CD8α hinge antibody was tested in an ADA assay using a bridging assay format based on MSD™ technology platform. During incubation, each Fab arm of the anti-CD8 hinge antibody binds to biotin and Ruthenium labeled CAR extracellular domain (ECD) respectively. The immunocomplex is captured on MSD™ assay plate coated by streptavidin through biotin-CAR ECD. The ruthenium label is in close enough proximity to the electrode to emit light upon electrochemical stimulation.

As shown in FIG. 7B, the mouse version of CD8B575 and human version anti-CD8 hinge antibodies were tested in the bridging format ADA assay. Each of these antibodies was serially diluted by 3-fold in neat serum starting from 10 μg/mL and characterized in the assay. The assay response was computed as the normalized value (NV) by dividing the raw electrochemiluminescence (ECL) signal from the sample by that derived from the negative control. The original mouse version of CD8B575 and CD8B575 showed strong binding in a dose dependent manner whereas other human version anti-CD8 hinge anti ids only generated very weak signals or no binding. Therefore, CD8B575 was selected as the positive control for the anti-KLK2 CAR ADA assay due to its strong binding to the CAR ECD.

As shown in FIG. 7C, the specificity of anti-CD8 hinge antibody, CD8B575, to CD8 hinge in CAR ECD was evaluated in a competition assay. The results indicate that the CD8 hinge-mFc specifically inhibited the binding of CD8B575 to labeled CAR ECD in the bridging format ADA assay. The assay signals were reduced to background under the inhibition of all three concentrations (20 ug/ml, 50 ug/ml and 100 ug/ml) of CD8 hinge-mFc whereas serial titrations of CD8B575 in the absence of CD8 hinge-mFc generated assay signal in a dose dependent manner.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed is:

1. An antibody that binds a CD8α hinge region, wherein the antibody is capable of binding to a functional exogenous receptor, and wherein the functional exogenous receptor comprises an extracellular domain, the CD8α hinge region, a transmembrane domain, and an intracellular signaling domain.

2. The antibody of claim 1, wherein the functional exogenous receptor is a T cell receptor (TCR), a chimeric antigen receptor (CAR), a chimeric TCR (cTCR), or a T cell antigen coupler (TAC)-like chimeric receptor.

3. The antibody of claim 1 or 2, wherein the functional exogenous receptor is a CAR.

4. The antibody of claim 1 or 2, wherein the functional exogenous receptor is a TCR.

5. The antibody of any one of claims 1 to 4, wherein the extracellular domain comprises an antigen binding domain derived from an antibody.

6. The antibody of any one of claims 1 to 4, wherein the extracellular domain comprises an antibody fragment.

7. The antibody of any one of claims 1 to 4, wherein the extracellular domain comprises a scFv.

8. The antibody of any one of claims 1 to 4, wherein the extracellular domain binds an antigen.

9. The antibody of claim 8, wherein the antigen is a tumor antigen.

10. The antibody of any one of claims 1 to 9, wherein the transmembrane domain is derived from a molecule selected from the group consisting of CD8α, CD4, CD28, CD137, CD80, CD86, CD152 and PD1.

11. The antibody of claim 10, wherein the transmembrane domain is from CD8α or CD28.

12. The antibody of any one of claims 1 to 11, wherein the intracellular signaling domain comprises a primary intracellular signaling domain of an immune effector cell.

13. The antibody of claim 12, wherein the primary intracellular signaling domain is from CD3ζ.

14. The antibody of any one of claims 1 to 13, wherein the intracellular signaling domain comprises a co-stimulatory signaling domain.

15. The antibody of claim 14, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

16. The antibody of claim 15, wherein the co-stimulatory signaling domain comprises a cytoplasmic domain of CD28 and/or a cytoplasmic domain of CD137.

17. The antibody of any one of claims 1 to 16, wherein the CD8α hinge region is located between the C-terminus of the extracellular domain and the N-terminus of the transmembrane domain.

18. The antibody of any one of claims 1 to 17, wherein the functional exogenous receptor further comprises a signal peptide.

19. The antibody of claim 18, wherein the signal peptide is from CD8α.

20. The antibody of any one of claims 1 to 19, wherein the antibody is capable of binding to an immune effector cell expressing the functional exogenous receptor, wherein optionally the immune effector cell does not express an endogenous CD8α, or the immune effector cell has been engineered to not express an endogenous CD8α.

21. The antibody of claim 20, wherein the immune effector cell is a T cell, a natural killer (NK) cell, a NK T cell, a macrophage, a peripheral blood mononuclear cell (PBMC), a monocyte, a neutrophil, or an eosinophil.

22. The antibody of claim 21, wherein the immune effector cell is a T cell and the T cell is a cytotoxic T cell, a helper T cell, a natural killer T cell, a αβ T cell, or a γδT cell; or wherein the immune effect cell is a NK cell.

23. The antibody of any one of claims 1 to 22, wherein the CD8α hinge region comprises an amino acid sequence of SEQ ID NO: 209.

24. The antibody of any one of claims 1 to 22, wherein the CD8α hinge region comprises an amino acid sequence of SEQ ID NO: 210.

25. The antibody of any one of claims 1 to 24, wherein the antibody comprises:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:27; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:28;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:54;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:79; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:80;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:106;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:131; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:x132;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:157; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:158; or
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:183; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:184.

26. The antibody of claim 25, wherein the VH CDR1, VH CDR2, VH CDR3, VL CDR1, VL CDR2, and VL CDR3 amino acid sequences are according to the Kabat numbering system.

27. The antibody of claim 25, wherein the VH CDR1, VH CDR2, VH CDR3, VL CDR1, VL CDR2, and VL CDR3 amino acid sequences are according to the Chothia numbering system.

28. The antibody of claim 25, wherein the VH CDR1, VH CDR2, VH CDR3, VL CDR1, VL CDR2, and VL CDR3 amino acid sequences are according to the AbM numbering system.

29. The antibody of claim 25, wherein the VH CDR1, VH CDR2, VH CDR3, VL CDR1, VL CDR2, and VL CDR3 amino acid sequences are according to the Contact numbering system.

30. The antibody of claim 25, wherein the VH CDR1, VH CDR2, VH CDR3, VL CDR1, VL CDR2, and VL CDR3 amino acid sequences are according to the IMGT numbering system.

31. The antibody of claim 25, wherein:
(i) the VH CDR1 comprises the amino acid sequence of SEQ ID NO:3, 9, 15, or 21, the VH CDR2 comprises the amino acid sequence of SEQ ID NO:4, 10, 16, or 22, the VH CDR3 comprises the amino acid sequence of SEQ ID NO:5, 11, 17, or 23, the VL CDR1 comprises the amino acid sequence of SEQ ID NO:6, 12, 18 or 24, the VL CDR2 comprises the amino acid sequence of SEQ ID NO:7, 13, 19, or 25, and the VL CDR3 comprises the amino acid sequence of SEQ ID NO:8, 14, 20, or 26;
(ii) the VH CDR1 comprises the amino acid sequence of SEQ ID NO:29, 35, 41, or 47, the VH CDR2 comprises the amino acid sequence of SEQ ID NO:30, 36, 42, or 48, the VH CDR3 comprises the amino acid sequence of SEQ ID NO:31, 37, 43, or 49, the VL CDR1 comprises the amino acid sequence of SEQ ID NO:32, 38, 44, or 50, the VL CDR2 comprises the amino acid sequence of SEQ ID NO:33, 39, 45, or 51, and the VL CDR3 comprises the amino acid sequence of SEQ ID NO:34, 40, 46, or 52;
(iii) the VH CDR1 comprises the amino acid sequence of SEQ ID NO:55, 61, 67, or 73, the VH CDR2 comprises the amino acid sequence of SEQ ID NO:56, 62, 68, or 74, the VH CDR3 comprises the amino acid sequence of SEQ ID NO:57, 63, 69, or 75, the VL CDR1 comprises the amino acid sequence of SEQ ID NO:58, 64, 70, or 76, the VL CDR2 comprises the amino acid sequence of SEQ ID NO:59, 65, 71, or 77, and the VL CDR3 comprises the amino acid sequence of SEQ ID NO:60, 66, 72, or 78;
(iv) the VH CDR1 comprises the amino acid sequence of SEQ ID NO:81, 87, 93, or 99, the VH CDR2 comprises the amino acid sequence of SEQ ID NO:82, 88, 94, or 100, the VH CDR3 comprises the amino acid sequence of SEQ ID NO:83, 89, 95, or 101, the VL CDR1 comprises the amino acid sequence of SEQ ID NO:84, 90, 96, or 102, the VL CDR2 comprises the amino acid sequence of SEQ ID NO:85, 91, 97, or 103, and the VL CDR3 comprises the amino acid sequence of SEQ ID NO:86, 92, 98, or 104;
(v) the VH CDR1 comprises the amino acid sequence of SEQ ID NO:107, 113, 119, or 125, the VH CDR2 comprises the amino acid sequence of SEQ ID NO:108, 114, 120, or 126, the VH CDR3 comprises the amino acid sequence of SEQ ID NO:109, 115, 121, or 127, the VL CDR1 comprises the amino acid sequence of SEQ ID NO:110, 116, 122, or 128, the VL CDR2 comprises the amino acid sequence of SEQ ID NO:111, 117, 123, or 129, and the VL CDR3 comprises the amino acid sequence of SEQ ID NO:112, 118, 124, or 130;
(vi) the VH CDR1 comprises the amino acid sequence of SEQ ID NO:133, 139, 145, or 151, the VH CDR2 comprises the amino acid sequence of SEQ ID NO:134, 140, 146, or 152, the VH CDR3 comprises the amino acid sequence of SEQ ID NO:135, 141, 147, or 153, the VL CDR1 comprises the amino acid sequence of SEQ ID NO:136, 142, 148, or 154, the VL CDR2 comprises the amino acid sequence of SEQ ID NO:137, 143, 149, or 155, and the VL CDR3 comprises the amino acid sequence of SEQ ID NO:138, 144, 150, or 156;
(vii) the VH CDR1 comprises the amino acid sequence of SEQ ID NO:159, 165, 171, or 177, the VH CDR2 comprises the amino acid sequence of SEQ ID NO:160, 166, 172, or 178, the VH CDR3 comprises the amino acid sequence of SEQ ID NO:161, 167, 173, or 179, the VL CDR1 comprises the amino acid sequence of SEQ ID NO:162, 168, 174, or 180, the VL CDR2 comprises the amino acid sequence of SEQ ID NO:163, 169, 175, or 181, and the VL CDR3 comprises the amino acid sequence of SEQ ID NO:164, 170, 176, or 182;
(viii) the VH CDR1 comprises the amino acid sequence of SEQ ID NO:185, 191, 197, or 203, the VH CDR2 comprises the amino acid sequence of SEQ ID NO:186, 192, 198, or 204, the VH CDR3 comprises the amino acid sequence of SEQ ID NO:187, 193, 199, or 205, the VL CDR1 comprises the amino acid sequence of SEQ ID NO:188, 194, 200, or 206, the VL CDR2 comprises the amino acid sequence of SEQ ID NO:189, 195, 201, or 207, and the VL CDR3 comprises the amino acid sequence of SEQ ID NO:190, 196, 202, or 208.

32. A nucleic acid encoding the antibody of any one of claims 1 to 31.

33. A vector comprising the nucleic acid of claim 32.

34. A host cell comprising the vector of claim 33.

35. An antibody that binds a CD8α hinge region, comprising:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:27; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:28;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:54;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:79; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:80;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:106;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:131; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:132;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:157; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:158; or
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:183; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:184.

36. The antibody of claim 35, wherein the VH CDR1, VH CDR2, VH CDR3, VL CDR1, VL CDR2, and VL CDR3 amino acid sequences are according to the Kabat numbering system.

37. The antibody of claim 35, wherein the VH CDR1, VH CDR2, VH CDR3, VL CDR1, VL CDR2, and VL CDR3 amino acid sequences are according to the Chothia numbering system.

38. The antibody of claim 35, wherein the VH CDR1, VH CDR2, VH CDR3, VL CDR1, VL CDR2, and VL CDR3 amino acid sequences are according to the AbM numbering system.

39. The antibody of claim 35, wherein the VH CDR1, VH CDR2, VH CDR3, VL CDR1, VL CDR2, and VL CDR3 amino acid sequences are according to the Contact numbering system.

40. The antibody of claim 35, wherein the VH CDR1, VH CDR2, VH CDR3, VL CDR1, VL CDR2, and VL CDR3 amino acid sequences are according to the IMGT numbering system.

41. The antibody of claim 35, wherein
(i) the VH CDR1 comprises the amino acid sequence of SEQ ID NO:3, 9, 15, or 21, the VH CDR2 comprises the amino acid sequence of SEQ ID NO:4, 10, 16, or 22, the VH CDR3 comprises the amino acid sequence of SEQ ID NO:5, 11, 17, or 23, the VL CDR1 comprises the amino acid sequence of SEQ ID NO:6, 12, 18 or 24, the VL CDR2 comprises the amino acid sequence of SEQ ID NO:7, 13, 19, or 25, and the VL CDR3 comprises the amino acid sequence of SEQ ID NO:8, 14, 20, or 26;
(ii) the VH CDR1 comprises the amino acid sequence of SEQ ID NO:29, 35, 41, or 47, the VH CDR2 comprises the amino acid sequence of SEQ ID NO:30, 36, 42, or 48, the VH CDR3 comprises the amino acid sequence of SEQ ID NO:31, 37, 43, or 49, the VL CDR1 comprises the amino acid sequence of SEQ ID NO:32, 38, 44, or 50, the VL CDR2 comprises the amino acid sequence of SEQ ID NO:33, 39, 45, or 51, and the VL CDR3 comprises the amino acid sequence of SEQ ID NO:34, 40, 46, or 52;
(iii) the VH CDR1 comprises the amino acid sequence of SEQ ID NO:55, 61, 67, or 73, the VH CDR2 comprises the amino acid sequence of SEQ ID NO:56, 62, 68, or 74, the VH CDR3 comprises the amino acid sequence of SEQ ID NO:57, 63, 69, or 75, the VL CDR1 comprises the amino acid sequence of SEQ ID NO:58, 64, 70, or 76, the VL CDR2 comprises the amino acid sequence of SEQ ID NO:59, 65, 71, or 77, and the VL CDR3 comprises the amino acid sequence of SEQ ID NO:60, 66, 72, or 78;
(iv) the VH CDR1 comprises the amino acid sequence of SEQ ID NO:81, 87, 93, or 99, the VH CDR2 comprises the amino acid sequence of SEQ ID NO:82, 88, 94, or 100, the VH CDR3 comprises the amino acid sequence of SEQ ID NO:83, 89, 95, or 101, the VL CDR1 comprises the amino acid sequence of SEQ ID NO:84, 90, 96, or 102, the VL CDR2 comprises the amino acid sequence of SEQ ID NO:85, 91, 97, or 103, and the VL CDR3 comprises the amino acid sequence of SEQ ID NO:86, 92, 98, or 104;
(v) the VH CDR1 comprises the amino acid sequence of SEQ ID NO:107, 113, 119, or 125, the VH CDR2 comprises the amino acid sequence of SEQ ID NO:108, 114, 120, or 126, the VH CDR3 comprises the amino acid sequence of SEQ ID NO:109, 115, 121, or 127, the VL CDR1 comprises the amino acid sequence of SEQ ID NO:110, 116, 122, or 128, the VL CDR2 comprises the amino acid sequence of SEQ ID NO:111, 117, 123, or 129, and the VL CDR3 comprises the amino acid sequence of SEQ ID NO:112, 118, 124, or 130;
(vi) the VH CDR1 comprises the amino acid sequence of SEQ ID NO:133, 139, 145, or 151, the VH CDR2 comprises the amino acid sequence of SEQ ID NO:134, 140, 146, or 152, the VH CDR3 comprises the amino acid sequence of SEQ ID NO:135, 141, 147, or 153, the VL CDR1 comprises the amino acid sequence of SEQ ID NO:136, 142, 148, or 154, the VL CDR2 comprises the amino acid sequence of SEQ ID NO:137, 143, 149, or 155, and the VL CDR3 comprises the amino acid sequence of SEQ ID NO:138, 144, 150, or 156;
(vii) the VH CDR1 comprises the amino acid sequence of SEQ ID NO:159, 165, 171, or 177, the VH CDR2 comprises the amino acid sequence of SEQ ID NO:160, 166, 172, or 178, the VH CDR3 comprises the amino acid sequence of SEQ ID NO:161, 167, 173, or 179, the VL CDR1 comprises the amino acid sequence of SEQ ID NO:162, 168, 174, or 180, the VL CDR2 comprises the amino acid sequence of SEQ ID NO:163, 169, 175, or 181, and the VL CDR3 comprises the amino acid sequence of SEQ ID NO:164, 170, 176, or 182;
(viii) the VH CDR1 comprises the amino acid sequence of SEQ ID NO:185, 191, 197, or 203, the VH CDR2 comprises the amino acid sequence of SEQ ID NO:186, 192, 198, or 204, the VH CDR3 comprises the amino acid sequence of SEQ ID NO:187, 193, 199, or 205, the VL CDR1 comprises the amino acid sequence of SEQ ID NO:188, 194, 200, or 206, the VL CDR2 comprises the amino acid sequence of SEQ ID NO:189, 195, 201, or 207, and the VL CDR3 comprises the amino acid sequence of SEQ ID NO:190, 196, 202, or 208.

42. The antibody of any one of claims 35 to 41, comprising:
(i) a VH comprising the amino acid sequence of SEQ ID NO: 1, and a VL comprising the amino acid sequence of SEQ ID NO: 2;
(ii) a VH comprising the amino acid sequence of SEQ ID NO: 27, and a VL comprising the amino acid sequence of SEQ ID NO: 28;
(iii) a VH comprising the amino acid sequence of SEQ ID NO: 53, and a VL comprising the amino acid sequence of SEQ ID NO: 54;
(iv) a VH comprising the amino acid sequence of SEQ ID NO:79, and a VL comprising the amino acid sequence of SEQ ID NO:80;
(v) a VH comprising the amino acid sequence of SEQ ID NO: 105, and a VL comprising the amino acid sequence of SEQ ID NO: 106;
(vi) a VH comprising the amino acid sequence of SEQ ID NO: 131, and a VL comprising the amino acid sequence of SEQ ID NO: 132;
(vii) a VH comprising the amino acid sequence of SEQ ID NO: 157, and a VL comprising the amino acid sequence of SEQ ID NO:158; or
(viii) a VH comprising the amino acid sequence of SEQ ID NO:183, and a VL comprising the amino acid sequence of SEQ ID NO:184.

43. The antibody of any one of claims 35 to 42, wherein the antibody is a humanized antibody.

44. The antibody of any one of claims 35 to 42, wherein the antibody is a human antibody.

45. The antibody of any one of claims 35 to 44, wherein the antibody is an IgG antibody.

46. The antibody of claim 45, wherein the IgG antibody is an IgG1, IgG2, IgG3, or IgG4 antibody.

47. The antibody of any one of claims 35 to 46, wherein the antibody comprises a kappa light chain.

48. The antibody of any one of claims 35 to 46, wherein the antibody comprises a lambda light chain.

49. The antibody of any one of claims 35 to 48, wherein the antibody is a monoclonal antibody.

50. The antibody of any one of claims 35 to 39, wherein the antibody is a multivalent antibody.

51. The antibody of any one of claims 35 to 49, wherein the antibody is a multispecific antibody.

52. The antibody of any one of claims 35 to 51, wherein the antibody is genetically fused to or chemically conjugated to an agent.

53. A nucleic acid encoding the antibody of any one of claims 35 to 52.

54. A vector comprising the nucleic acid of claim 53.

55. A host cell comprising the vector of claim 54.

56. A kit comprising the antibody of any one of claims 1 to 31 and 35 to 52.

57. A method for detecting and/or enriching an agent comprising a CD8α hinge region in a system comprising contacting the system with an antibody that binds a CD8α hinge region.

58. A method for detecting and/or enriching a functional exogenous receptor in a system comprising contacting the system with an antibody that binds a CD8α hinge region, wherein the functional exogenous receptor comprises an extracellular domain, the CD8α hinge region, a transmembrane domain, and an intracellular signaling domain, wherein optionally the functional exogenous receptor is expressed in an immune effector cell.

59. A method for detecting and/or enriching immune effector cells expressing a functional exogenous receptor comprising contacting a population of cells with an antibody that binds a CD8α hinge region, wherein the functional exogenous receptor comprises an extracellular domain, the CD8α hinge region, a transmembrane domain, and an intracellular signaling domain.

60. The method of claim 58 or claim 59, the functional exogenous receptor is a T cell receptor (TCR), a chimeric antigen receptor (CAR), a chimeric TCR (cTCR), or a T cell antigen coupler (TAC)-like chimeric receptor.

61. The method of any one of claims 57 to 60, wherein the antibody that binds the CD8α hinge region is the antibody of any one of claims 1 to 31 and 35 to 52.

62. A system comprising a means for binding a CD8α hinge region in a functional exogenous receptor, wherein the functional exogenous receptor comprises an extracellular domain, the CD8α hinge region, a transmembrane domain, and an intracellular signaling domain.

63. A system comprising a means for binding a CD8α hinge region in a functional exogenous receptor expressed in an immune effector cell, wherein the functional exogenous receptor comprises an extracellular domain, the CD8α hinge region, a transmembrane domain, and an intracellular signaling domain.

64. A system comprising a means for binding an immune effector cell expressing a functional exogenous receptor, wherein the functional exogenous receptor comprises an extracellular domain, a CD8α hinge region, a transmembrane domain, and an intracellular signaling domain.

65. The system of any one of claims 62 to 64, wherein the functional exogenous receptor is a T cell receptor (TCR), a chimeric antigen receptor (CAR), a chimeric TCR (cTCR), or a T cell antigen coupler (TAC)-like chimeric receptor.

66. The system of claim 65, wherein the functional exogenous receptor is a CAR.

67. The system of claim 65, wherein the functional exogenous receptor is a TCR.

68. The system of any one of claims 62 to 67, wherein the immune effector cell does not express an endogenous CD8α, or the immune effector cell has been engineered to not express an endogenous CD8α.

69. The system of claim 68, wherein the immune effector cell is a T cell, a natural killer (NK) cell, a NK T cell, a macrophage, a peripheral blood mononuclear cell (PBMC), a monocyte, a neutrophil, or an eosinophil.

70. The system of claim 69, wherein the immune effector cell is a T cell and the T cell is a cytotoxic T cell, a helper T cell, a natural killer T cell, a αβ T cell, or a γδT cell.

71. The system of claim 69, wherein the immune effect cell is a NK cell.

72. A method for detecting and/or enriching a functional exogenous receptor using the system of any one of claims 62 to 71.

73. A method for detecting and/or enriching immune effector cells expressing a functional exogenous receptor using the system of any one of claims 62 to 71.
